SEQUENCE LISTING (161 sequences)

SEQ ID NO: 1 - DNA, 50 bp, synthetic construct
  source 1..50: mol_type = other DNA
gcgctctggcaaaccgaaaaggatgactggacggtcccttatggccgcat

SEQ ID NO: 2 - DNA, 50 bp, synthetic construct
  source 1..50: mol_type = other DNA
cccagtgggatttcattccctagcgtggtggatcacccaatgccagaaga

SEQ ID NO: 3 - DNA, 50 bp, synthetic construct
  source 1..50: mol_type = other DNA
gcccgtcaagtgctgcccctgcctgtgtctgggtttctgttggctgtttt

SEQ ID NO: 4 - DNA, 50 bp, synthetic construct
  source 1..50: mol_type = other DNA
ggggtaacacagagtgcccttatgaaggagttggagatcctgcaaggaag

SEQ ID NO: 5 - DNA, 50 bp, synthetic construct
  source 1..50: mol_type = other DNA
cctcttgagctggaacgcctgaaactggagcctcacgaagggctgctgct

SEQ ID NO: 6 - DNA, 50 bp, synthetic construct
  source 1..50: mol_type = other DNA
ctgctgcgactgatgccaggacaacctttctcccagatgtaaacagagag

SEQ ID NO: 7 - DNA, 50 bp, synthetic construct
  source 1..50: mol_type = other DNA
atgatggcggtggaggtggtggttgtagtgtgatggatcccctttaggtt

SEQ ID NO: 8 - DNA, 50 bp, synthetic construct
  source 1..50: mol_type = other DNA
gggagtcacttgatgctttcaggttaatcagagctatgggtgctacaggc

SEQ ID NO: 9 - DNA, 50 bp, synthetic construct
  source 1..50: mol_type = other DNA
ccaagttcatcctgggctccattgggtctgccattgcggctgtcattgcg

SEQ ID NO: 10 - DNA, 50 bp, synthetic construct
  source 1..50: mol_type = other DNA
gttcactgactataggctactgcagcactggggtgtcagttgttggtccc

SEQ ID NO: 11 - DNA, 50 bp, synthetic construct
  source 1..50: mol_type = other DNA
tatcgacgtcgtgatccagaagtccagagctgagaaggactgcctgtttg

SEQ ID NO: 12 - DNA, 50 bp, synthetic construct
  source 1..50: mol_type = other DNA
agtggcgaggatggcaagaaaagctggcaacttctatgtacctgcagaac

SEQ ID NO: 13 - DNA, 50 bp, synthetic construct
  source 1..50: mol_type = other DNA
caagcaggtttgtctgagagggatggtgctgggggaaggctttggattgc

SEQ ID NO: 14 - DNA, 50 bp, synthetic construct
  source 1..50: mol_type = other DNA
gggtcctatggtggtccattgctcagactgtcatcctcatcctcactggc

SEQ ID NO: 15 - DNA, 50 bp, synthetic construct
  source 1..50: mol_type = other DNA
gggatttagccaagagcacagacttggattccttctgtccctccccacct

SEQ ID NO: 16 - DNA, 50 bp, synthetic construct
  source 1..50: mol_type = other DNA
catgctcaacacaaaggagctgggcaacgagtccaacttcagtgatgcac

SEQ ID NO: 17 - DNA, 50 bp, synthetic construct
  source 1..50: mol_type = other DNA
gcattgcctactccagcagccgtgaaggactctctgctgtttttcctctg

SEQ ID NO: 18 - DNA, 50 bp, synthetic construct
  source 1..50: mol_type = other DNA
ataggtccaatctgctctcaaggccttggtcctggtgggattccttcacc

SEQ ID NO: 19 - DNA, 50 bp, synthetic construct
  source 1..50: mol_type = other DNA
tttcaaggttctaaccctatactgtagtattctttggggtgcccctctcc

SEQ ID NO: 20 - DNA, 50 bp, synthetic construct
  source 1..50: mol_type = other DNA
gtaacagggtgcagtgttgtttatacttcattgctccttcaggacatggg

SEQ ID NO: 21 - DNA, 50 bp, synthetic construct
  source 1..50: mol_type = other DNA
gagctgccgggcaacctcagatgaccgacttttccctttgagcctcagtt

SEQ ID NO: 22 - DNA, 50 bp, synthetic construct
  source 1..50: mol_type = other DNA
caacagccgccaccacagcagcctgatgcccatcagcagcttccccccag

SEQ ID NO: 23 - DNA, 50 bp, synthetic construct
  source 1..50: mol_type = other DNA
tctgaggtcctggaggtgccatttccctctctgttgaagcaaaggccggg

SEQ ID NO: 24 - DNA, 50 bp, synthetic construct
  source 1..50: mol_type = other DNA
tgatgaagagactgctcctcccgttattgccccgcgaccggatcatacga

SEQ ID NO: 25 - DNA, 50 bp, synthetic construct
  source 1..50: mol_type = other DNA
tagacactccccgaagtcttttgttcgcatggtcacacactgatgcttag

SEQ ID NO: 26 - DNA, 50 bp, synthetic construct
  source 1..50: mol_type = other DNA
cggagtagattccgggtgcctttactccactgtgacctcataaatccagg

SEQ ID NO: 27 - DNA, 50 bp, synthetic construct
  source 1..50: mol_type = other DNA
ttgaatacttattgtttggcaggtcatccacacacttctgcccccactgc

SEQ ID NO: 28 - DNA, 50 bp, synthetic construct
  source 1..50: mol_type = other DNA
atcatctcgatctggttgtgactgtggcccctgccgtgtctcctcgtcag

SEQ ID NO: 29 - DNA, 50 bp, synthetic construct
  source 1..50: mol_type = other DNA
cacccccgagctcgcatgctgtcacccattccagcctaaatgtgaccata

SEQ ID NO: 30 - DNA, 50 bp, synthetic construct
  source 1..50: mol_type = other DNA
ttgttttatttggggctgggggaggtatatgatgagcagacttctcggaa

SEQ ID NO: 31 - DNA, 50 bp, synthetic construct
  source 1..50: mol_type = other DNA
ggaatactgaagaagtgggaaggaaccaagctgacacaggcctcactgca

SEQ ID NO: 32 - DNA, 50 bp, synthetic construct
  source 1..50: mol_type = other DNA
acctgtgtgaagcagcagccagccttaaagtagtccattcttgctaatgg

SEQ ID NO: 33 - DNA, 50 bp, synthetic construct
  source 1..50: mol_type = other DNA
ccagtgtctactatttagtgccctggctctatttcggtcctcctccccgg

SEQ ID NO: 34 - DNA, 50 bp, synthetic construct
  source 1..50: mol_type = other DNA
tggctcccaactcctccctatcctaaaggcccactggcattaaagtgctg

SEQ ID NO: 35 - DNA, 50 bp, synthetic construct
  source 1..50: mol_type = other DNA
tgcgccgacgatgcccagaatccagaactttgtctatcactctccccaac

SEQ ID NO: 36 - DNA, 50 bp, synthetic construct
  source 1..50: mol_type = other DNA
cacttaccacctcatttgcttttctggaggccatgcaatataggcgggac

SEQ ID NO: 37 - DNA, 50 bp, synthetic construct
  source 1..50: mol_type = other DNA
gcaacagcggcaaactactgggccaagatgagcaaccccacatttttggg

SEQ ID NO: 38 - DNA, 50 bp, synthetic construct
  source 1..50: mol_type = other DNA
ctcccacccctcctagcacccccacatcccccatctagtaacatctcaga

SEQ ID NO: 39 - DNA, 50 bp, synthetic construct
  source 1..50: mol_type = other DNA
catcatggaggacctggacacaaatgcagacaagcagctgagcttcgagg

SEQ ID NO: 40 - DNA, 50 bp, synthetic construct
  source 1..50: mol_type = other DNA
ccgaggaaaccttgcccccatcactcagcattgcacttagatacagaatg

SEQ ID NO: 41 - DNA, 50 bp, synthetic construct
  source 1..50: mol_type = other DNA
gaggtggtggggaccagggctatgggagtggcaggtattatgacagtcga

SEQ ID NO: 42 - DNA, 50 bp, synthetic construct
  source 1..50: mol_type = other DNA
ggtccagagtacttgttttcccgatgtgtccagccagctccgcagcagct

SEQ ID NO: 43 - DNA, 50 bp, synthetic construct
  source 1..50: mol_type = other DNA
aagcggatggcagcactgaagaacaagccccaggttccagttccagggtc

SEQ ID NO: 44 - DNA, 50 bp, synthetic construct
  source 1..50: mol_type = other DNA
cccagtcttgtggatggaaatgtagtgctcgagtcacattctgcttaaag

SEQ ID NO: 45 - DNA, 50 bp, synthetic construct
  source 1..50: mol_type = other DNA
atctctggctgagggatgacttacctgttcagtactctacaattcctctg

SEQ ID NO: 46 - DNA, 50 bp, synthetic construct
  source 1..50: mol_type = other DNA
ccaacaggagctgtgcagattgtaaagccccagatcctgactgggcatcc

SEQ ID NO: 47 - DNA, 50 bp, synthetic construct
  source 1..50: mol_type = other DNA
cattggacggcagcaactgggggcaagaacaacgggaagatcacctgtgc

SEQ ID NO: 48 - DNA, 50 bp, synthetic construct
  source 1..50: mol_type = other DNA
ggaagccaggtgcctttaatccactgtaacctcacaactccaagtccaca

SEQ ID NO: 49 - DNA, 50 bp, synthetic construct
  source 1..50: mol_type = other DNA
gtggcgaccaagacgattttctgccttagagcaagggattcaccctccgc

SEQ ID NO: 50 - DNA, 50 bp, synthetic construct
  source 1..50: mol_type = other DNA
ggcctagtacccccgcgtggcctatccactcctcacatccacacactgca

SEQ ID NO: 51 - DNA, 50 bp, synthetic construct
  source 1..50: mol_type = other DNA
atggtgggcatccagggctctgacagcgtcttctccggcttcctgctctt

SEQ ID NO: 52 - DNA, 50 bp, synthetic construct
  source 1..50: mol_type = other DNA
taggccctgaactttctccagccaagctctgagggcccacctcctgcctc

SEQ ID NO: 53 - DNA, 50 bp, synthetic construct
  source 1..50: mol_type = other DNA
cagcccagccctgtgaaagatggagctgacttgctgcaggggacctgatt

SEQ ID NO: 54 - DNA, 50 bp, synthetic construct
  source 1..50: mol_type = other DNA
catgaacagccgtgagtatggggcctggaagcagcaggtggaatccaaga

SEQ ID NO: 55 - DNA, 50 bp, synthetic construct
  source 1..50: mol_type = other DNA
cttgtcaaggtgaagggacacaagttgccagccaccaactttcttgcctc

SEQ ID NO: 56 - DNA, 50 bp, synthetic construct
  source 1..50: mol_type = other DNA
tgaggcccaaagcacttgcttacatcctctgatagctgtttcaaatgtgc

SEQ ID NO: 57 - DNA, 50 bp, synthetic construct
  source 1..50: mol_type = other DNA
cagaccagtggccaacagccccaaggggagcccaattaaatacctgggtt

SEQ ID NO: 58 - DNA, 50 bp, synthetic construct
  source 1..50: mol_type = other DNA
gctgagagctgggctttcctctgtgaccatcccggcctgtaacatatctg

SEQ ID NO: 59 - DNA, 50 bp, synthetic construct
  source 1..50: mol_type = other DNA
cagaagctacagctgccaggtcatgcacgaagggagcaccgtggagaaga

SEQ ID NO: 60 - DNA, 50 bp, synthetic construct
  source 1..50: mol_type = other DNA
tcctggactctaccctcctctgaaagaagctggggcttgctctgacggtc

SEQ ID NO: 61 - DNA, 50 bp, synthetic construct
  source 1..50: mol_type = other DNA
tcaacaaatacgaatgtgcagcggtgctggggtcatgatggctcggtggg

SEQ ID NO: 62 - DNA, 50 bp, synthetic construct
  source 1..50: mol_type = other DNA
cgtggctaggcctttcctgccgagtgctctgatgcaatagtggaaatcgc

SEQ ID NO: 63 - DNA, 50 bp, synthetic construct
  source 1..50: mol_type = other DNA
ccaggaccaaggctcagggatgagtgggagagatgggttatactgtccct

SEQ ID NO: 64 - DNA, 50 bp, synthetic construct
  source 1..50: mol_type = other DNA
tgcccttgctgtcgctgacggtgactgcgggaggccaggttgtttttcat

SEQ ID NO: 65 - DNA, 50 bp, synthetic construct
  source 1..50: mol_type = other DNA
ctgggccctgtaaaatagtgttactgtaatactctgttttgcctcctgcc

SEQ ID NO: 66 - DNA, 50 bp, synthetic construct
  source 1..50: mol_type = other DNA
gggggtgtccatgtggcgtgtgtgtgagtgagacttttttactgcgtccc

SEQ ID NO: 67 - DNA, 50 bp, synthetic construct
  source 1..50: mol_type = other DNA
ggactgggaaaatctgcagcatcagactatgcctttcatcccccagccag

SEQ ID NO: 68 - DNA, 50 bp, synthetic construct
  source 1..50: mol_type = other DNA
cctcactgtggaacccctcaatggactctctcaacccagggacacccaag

SEQ ID NO: 69 - DNA, 50 bp, synthetic construct
  source 1..50: mol_type = other DNA
cagatgtggtattgccctggatgcatggatgtttccactgggtgatgaag

SEQ ID NO: 70 - DNA, 50 bp, synthetic construct
  source 1..50: mol_type = other DNA
caaaggggcatcggagaagtgcagctgctgtgcctgatgtgggaacagct

SEQ ID NO: 71 - DNA, 50 bp, synthetic construct
  source 1..50: mol_type = other DNA
gcttctactggcgcgtgagttcccggagtgagttgaaccaggtggaccaa

SEQ ID NO: 72 - DNA, 50 bp, synthetic construct
  source 1..50: mol_type = other DNA
ctgcgaggcttacctggtggggctctttgaggacaccaacctgtgtgcta

SEQ ID NO: 73 - DNA, 50 bp, synthetic construct
  source 1..50: mol_type = other DNA
tcaagaagccccatcgctatcggcctggtacagtggctctccgcgagatt

SEQ ID NO: 74 - DNA, 50 bp, synthetic construct
  source 1..50: mol_type = other DNA
aaagaagttctgcaagaaggactatgcggtgcaggtggcggtgggtgcgc

SEQ ID NO: 75 - DNA, 50 bp, synthetic construct
  source 1..50: mol_type = other DNA
tgggtggaaacctaccctgtgggcacccatgtccgagccccctggcattt

SEQ ID NO: 76 - DNA, 50 bp, synthetic construct
  source 1..50: mol_type = other DNA
ctgtccttaagtgccatcaactctcagctcccttgtagcttttgtgggat

SEQ ID NO: 77 - DNA, 50 bp, synthetic construct
  source 1..50: mol_type = other DNA
tgctggcaccaccaggcacctcacagaaatgagatcaggctctgcctcac

SEQ ID NO: 78 - DNA, 50 bp, synthetic construct
  source 1..50: mol_type = other DNA
ctactcttctcccttctttcatcttggggttgggtagagaaacacaaagg

SEQ ID NO: 79 - DNA, 50 bp, synthetic construct
  source 1..50: mol_type = other DNA
aattgctggaggaagagccatgagccgaggaatgcagacagcctcttctc

SEQ ID NO: 80 - DNA, 50 bp, synthetic construct
  source 1..50: mol_type = other DNA
ttaccctgcctgagattcctcaggagaaaaggcaacctgcctccagcctg

SEQ ID NO: 81 - DNA, 50 bp, synthetic construct
  source 1..50: mol_type = other DNA
ggcttgcctttagatcgggtgctgatattgcctgtttcctagtaatgggc

SEQ ID NO: 82 - DNA, 50 bp, synthetic construct
  source 1..50: mol_type = other DNA
tggtgagatccttctcctacatgaggccagtctgacaagtctgggagagg

SEQ ID NO: 83 - DNA, 50 bp, synthetic construct
  source 1..50: mol_type = other DNA
cacctttgtgcacgagcccctggtagatgtccagaatgtctgtttccagg

SEQ ID NO: 84 - DNA, 50 bp, synthetic construct
  source 1..50: mol_type = other DNA
cccactggggggttggggtaatattctgtggtcctcagccctgtacctta

SEQ ID NO: 85 - DNA, 50 bp, synthetic construct
  source 1..50: mol_type = other DNA
tgttcttccccatgtcctggatgccactggaagtgcacactgcttgtatg

SEQ ID NO: 86 - DNA, 50 bp, synthetic construct
  source 1..50: mol_type = other DNA
acctccacgatttcgccaccaggaacacaaacagcttctaaagaggggtc

SEQ ID NO: 87 - DNA, 50 bp, synthetic construct
  source 1..50: mol_type = other DNA
cctgccaacgtcagtgccacactggtgtctgaaaggaaaaacaggaacag

SEQ ID NO: 88 - DNA, 50 bp, synthetic construct
  source 1..50: mol_type = other DNA
gctaagctggaagggtgcttatccctgcgtagaaacgcctgccaatgctt

SEQ ID NO: 89 - DNA, 50 bp, synthetic construct
  source 1..50: mol_type = other DNA
gctttggtattctgcttccacatgtgccagttgcctggattgggagccat

SEQ ID NO: 90 - DNA, 50 bp, synthetic construct
  source 1..50: mol_type = other DNA
taccacgccctgctggacagggaggagagcggctgatggagaaacttcct

SEQ ID NO: 91 - DNA, 50 bp, synthetic construct
  source 1..50: mol_type = other DNA
attatggctgttaaggccgggcgggtgactcaggcaggtaatcccagaac

SEQ ID NO: 92 - DNA, 50 bp, synthetic construct
  source 1..50: mol_type = other DNA
tcccccttcggatgtggcttgagctgtaggcgcggagggccggagacgct

SEQ ID NO: 93 - DNA, 50 bp, synthetic construct
  source 1..50: mol_type = other DNA
ttcccgctcccccatatcaattttccttttgtctccggggctgagtaaat

SEQ ID NO: 94 - DNA, 50 bp, synthetic construct
  source 1..50: mol_type = other DNA
gcccgatctttctaaaatacagttctgaatatgctgctcatccccacctg

SEQ ID NO: 95 - DNA, 50 bp, synthetic construct
  source 1..50: mol_type = other DNA
ccctgcaggcccaggctgagccactccaggcaagagctgatgaggttgct

SEQ ID NO: 96 - DNA, 50 bp, synthetic construct
  source 1..50: mol_type = other DNA
gaaccttatgtaccgagcaaatgccaggtctagcaaacataatgctagtc

SEQ ID NO: 97 - DNA, 50 bp, synthetic construct
  source 1..50: mol_type = other DNA
gctgtcccttcccagcctttgaggagttagggaagcataaagggggcttg

SEQ ID NO: 98 - DNA, 50 bp, synthetic construct
  source 1..50: mol_type = other DNA
ccggagacccctggaatttaaatcattagcaccgcgcccttccccgaaga

SEQ ID NO: 99 - DNA, 50 bp, synthetic construct
  source 1..50: mol_type = other DNA
cggtaaagcctacactcgttccagtcaccttactcgccatgaaagaagtc

SEQ ID NO: 100 - DNA, 50 bp, synthetic construct
  source 1..50: mol_type = other DNA
ccttccagttgtcttgaacagcctgactcctggcagccctatggaagttc

SEQ ID NO: 101 - DNA, 50 bp, synthetic construct
  source 1..50: mol_type = other DNA
ggcttggccaccctgccgctgcccagccacatcccttggttttgtatttt

SEQ ID NO: 102 - DNA, 50 bp, synthetic construct
  source 1..50: mol_type = other DNA
gcaaaagcccaaaggttcctaagcctggctgcaaagaagaatcaacaggg

SEQ ID NO: 103 - DNA, 50 bp, synthetic construct
  source 1..50: mol_type = other DNA
ctgggctaggaaagagagaacaagcaagccggggctacctgcccccaggt

SEQ ID NO: 104 - DNA, 50 bp, synthetic construct
  source 1..50: mol_type = other DNA
ctgagatcagcccatgtctctggtcgctctcttgcctgtggatcctgtct

SEQ ID NO: 105 - DNA, 50 bp, synthetic construct
  source 1..50: mol_type = other DNA
tccctgctctgggagcattgctagccttctaccccatccctggatccaca

SEQ ID NO: 106 - DNA, 50 bp, synthetic construct
  source 1..50: mol_type = other DNA
ccctagtctgctagccaggatccacaagtccttgttccactgtgccttgg

SEQ ID NO: 107 - DNA, 50 bp, synthetic construct
  source 1..50: mol_type = other DNA
ccagcccaccctaggagtgtttggatttgatcatggatgtagcatacacc

SEQ ID NO: 108 - DNA, 50 bp, synthetic construct
  source 1..50: mol_type = other DNA
cctttcacacttgtggctgatgtgaatgctgaccgaaacatggagatccc

SEQ ID NO: 109 - DNA, 50 bp, synthetic construct
  source 1..50: mol_type = other DNA
tgccccacaccctcccttaccctcctccagaccattcaggacacagggaa

SEQ ID NO: 110 - DNA, 50 bp, synthetic construct
  source 1..50: mol_type = other DNA
tggcctgggctttctgaggccttatctgatgcctctgcagttcatgtccc

SEQ ID NO: 111 - DNA, 50 bp, synthetic construct
  source 1..50: mol_type = other DNA
gccactgcctgctgcagagaaaacacactcctttgcttagcccacaaggt

SEQ ID NO: 112 - DNA, 50 bp, synthetic construct
  source 1..50: mol_type = other DNA
gaaaaatccaggtgccggaggagcagaaggatgagaaatactggagccgg

SEQ ID NO: 113 - DNA, 50 bp, synthetic construct
  source 1..50: mol_type = other DNA
gctgaggggtaagaggttgttgtagttgtcctggtgcctccatcagactc

SEQ ID NO: 114 - DNA, 50 bp, synthetic construct
  source 1..50: mol_type = other DNA
gcctgccggatgatgaatggcatgaagctgagtggccgagagattgacgt

SEQ ID NO: 115 - DNA, 50 bp, synthetic construct
  source 1..50: mol_type = other DNA
cgggaagctgaggggaaaccagtacaagaagatgatgaccaaagaggagc

SEQ ID NO: 116 - DNA, 50 bp, synthetic construct
  source 1..50: mol_type = other DNA
gcctcctgatccagccggggcccagattccactgaggttagagtccattt

SEQ ID NO: 117 - DNA, 50 bp, synthetic construct
  source 1..50: mol_type = other DNA
ccgccattgggctactgtagatttgtatccttgatgaatctggggtttcc

SEQ ID NO: 118 - DNA, 50 bp, synthetic construct
  source 1..50: mol_type = other DNA
catcctttcggctgtggtgcaggtgaatggcacgcccgtaatgctgcaga

SEQ ID NO: 119 - DNA, 50 bp, synthetic construct
  source 1..50: mol_type = other DNA
cttgacatgagcacctttagatcccttcccctccatgggctttgggccac

SEQ ID NO: 120 - DNA, 50 bp, synthetic construct
  source 1..50: mol_type = other DNA
gccctgggggtttcagtcatctccccatttggtcccctggactgtctttg

SEQ ID NO: 121 - DNA, 50 bp, synthetic construct
  source 1..50: mol_type = other DNA
gaaccactgagtcaggagagccaggtggaggaaccaccgagtcaggagag

SEQ ID NO: 122 - DNA, 50 bp, synthetic construct
  source 1..50: mol_type = other DNA
acgctagggactggttccagggccacacatataccaaaatctgcccatac

SEQ ID NO: 123 - DNA, 50 bp, synthetic construct
  source 1..50: mol_type = other DNA
caagataagcatttctttcctgagttcaggtgactgaggaagagccacaa

SEQ ID NO: 124 - DNA, 50 bp, synthetic construct
  source 1..50: mol_type = other DNA
gtctttgcacttacacacactcttcctgttctgccttcacctatgccggg

SEQ ID NO: 125 - DNA, 50 bp, synthetic construct
  source 1..50: mol_type = other DNA
gcgtagtttggtggaaaaacgggccttgcgtcggcctcacccccagtgtt

SEQ ID NO: 126 - DNA, 50 bp, synthetic construct
  source 1..50: mol_type = other DNA
gccgctccagctttgcacgtttcgatcccaaaggccctttttagggccga

SEQ ID NO: 127 - DNA, 50 bp, synthetic construct
  source 1..50: mol_type = other DNA
ctcctgcttcctccctgccattcatccctgcccctctccatgaagcttga

SEQ ID NO: 128 - DNA, 50 bp, synthetic construct
  source 1..50: mol_type = other DNA
cacacagccacagttgccaaatcccgtactccttgccaccggctttctag

SEQ ID NO: 129 - DNA, 50 bp, synthetic construct
  source 1..50: mol_type = other DNA
cctgccctgggttctgagttgctgagtggagacgatgatggagagcagtg

SEQ ID NO: 130 - DNA, 50 bp, synthetic construct
  source 1..50: mol_type = other DNA
cttgctatagaagacctgggacagaggactgctgtctgccctctctggtc

SEQ ID NO: 131 - DNA, 50 bp, synthetic construct
  source 1..50: mol_type = other DNA
tgcctttgccctccctttacactgtctgccggcgtcaacaagcgacacag

SEQ ID NO: 132 - DNA, 50 bp, synthetic construct
  source 1..50: mol_type = other DNA
ctggtgcctgtctcttgcttaccaatgtctaaggtccccactgcctgctg

SEQ ID NO: 133 - DNA, 50 bp, synthetic construct
  source 1..50: mol_type = other DNA
aagaggctgcggtgagccaatttagagcccaaagagccccgagggaacct

SEQ ID NO: 134 - DNA, 50 bp, synthetic construct
  source 1..50: mol_type = other DNA
ataccggcttccagagaccccttttctccagccatattacatcaggctag

SEQ ID NO: 135 - DNA, 50 bp, synthetic construct
  source 1..50: mol_type = other DNA
cccagggcaggtttgcttgacctctgcctcagttctcgactctaaaggac

SEQ ID NO: 136 - DNA, 50 bp, synthetic construct
  source 1..50: mol_type = other DNA
cctcccagtgtattctgcattgtccttaccctagatcagccccttctgtg

SEQ ID NO: 137 - DNA, 50 bp, synthetic construct
  source 1..50: mol_type = other DNA
accagacatcttgaccattcagctagaaccctggcagcaacagagctatt

SEQ ID NO: 138 - DNA, 50 bp, synthetic construct
  source 1..50: mol_type = other DNA
tccagtctgtcaccctcctttcctgctcccatacacccaaggcttgtttc

SEQ ID NO: 139 - DNA, 50 bp, synthetic construct
  source 1..50: mol_type = other DNA
ccaaggcacacagggtactttctggacccactgctggacagacttgaagg

SEQ ID NO: 140 - DNA, 50 bp, synthetic construct
  source 1..50: mol_type = other DNA
tctgtggaattggtgtccctgttagcaatggcagagaccagcctggagcc

SEQ ID NO: 141 - DNA, 50 bp, synthetic construct
  source 1..50: mol_type = other DNA
ggttcctgtagcagcagccgaagggagacctcgcttaaaccaacacaatc

SEQ ID NO: 142 - DNA, 50 bp, synthetic construct
  source 1..50: mol_type = other DNA
ccctgctccagtcacacccggaagctgactggtccacgcacagctgaagc

SEQ ID NO: 143 - DNA, 50 bp, synthetic construct
  source 1..50: mol_type = other DNA
aggagtcgctctagctggtacccgtaaaagttgtgggaattgtgaccccc

SEQ ID NO: 144 - DNA, 50 bp, synthetic construct
  source 1..50: mol_type = other DNA
tctgggttcaaattctgaaactgtcacttattacctgtatgaacatgggc

SEQ ID NO: 145 - DNA, 50 bp, synthetic construct
  source 1..50: mol_type = other DNA
ctctgtgcaacgccacctttaaaaagaccttccggcacctgctgctgtgc

SEQ ID NO: 146 - DNA, 50 bp, synthetic construct
  source 1..50: mol_type = other DNA
ttgtgaacgaccacccctgggattctggtgctgatggcggtacttcggtt

SEQ ID NO: 147 - DNA, 50 bp, synthetic construct
  source 1..50: mol_type = other DNA
gatgtgatagagtgtgtggggctctgtgtccttccctgggagctggcatt

SEQ ID NO: 148 - DNA, 50 bp, synthetic construct
  source 1..50: mol_type = other DNA
gccagcatggtctgtgtttccttgtggattcacctgtggccctgctgtgg

SEQ ID NO: 149 - DNA, 50 bp, synthetic construct
  source 1..50: mol_type = other DNA
catggacagaagaaggcagcaggtggcagaatggggtgcatgaaggtttc

SEQ ID NO: 150 - DNA, 50 bp, synthetic construct
  source 1..50: mol_type = other DNA
gaggtctagaagtacctaaacctgctgttccacagccagtcccttcaagc

SEQ ID NO: 151 - DNA, 50 bp, synthetic construct
  source 1..50: mol_type = other DNA
tcatatatcacccgtgtggggaccttccgggcacacagcagccctgagat

SEQ ID NO: 152 - DNA, 50 bp, synthetic construct
  source 1..50: mol_type = other DNA
tggctgggccatgtagactgcctcctccatgagtttgccagctttttgag

SEQ ID NO: 153 - DNA, 50 bp, synthetic construct
  source 1..50: mol_type = other DNA
cggtagtcaacgggtctgctttcccctcgggatcaactcttccaggacca

SEQ ID NO: 154 - DNA, 50 bp, synthetic construct
  source 1..50: mol_type = other DNA
ccctgactttgtttctgcaaaggcacctgcatgtgtctgtgttcgtgtag

SEQ ID NO: 155 - DNA, 50 bp, synthetic construct
  source 1..50: mol_type = other DNA
gcaaaagggccagtcgcgtttctatttctctcgatcccaggcttctgcgg

SEQ ID NO: 156 - DNA, 50 bp, synthetic construct
  source 1..50: mol_type = other DNA
tgccccgctgaatctcctggctgactttgctggtggtggccttatgtgtg

SEQ ID NO: 157 - DNA, 50 bp, synthetic construct
  source 1..50: mol_type = other DNA
tgccactgcttgggcagagtaaaaggtgccaggaggagcatgggtgtgga

SEQ ID NO: 158 - DNA, 50 bp, synthetic construct
  source 1..50: mol_type = other DNA
cggccaaacatccctggactagaagttgcaggggaaatcaaacagcccag

SEQ ID NO: 159 - DNA, 50 bp, synthetic construct
  source 1..50: mol_type = other DNA
ccctacaatggagcacaggactggatttactctggcagctactgggacag

SEQ ID NO: 160 - DNA, 50 bp, synthetic construct
  source 1..50: mol_type = other DNA
ccccaagacctaagggttttatctcctccccttgaatatgggtggctctg

SEQ ID NO: 161 - DNA, 50 bp, synthetic construct
  source 1..50: mol_type = other DNA
ggtaaagggatttggggatgcgtggctgggtggttctgtctccaggtctc

(19) United States
(12) Patent Application Publication
(10) Pub. No.: US 2026/0275417 A1
(43) Pub. Date: Sep. 17, 2026

(54) GENE SIGNATURE

(71) Applicant: IMPERIAL COLLEGE INNOVATIONS LIMITED, London (GB)

(72) Inventors: Michael LEVIN, London (GB); Aubrey CUNNINGTON, London (GB); Myrsini KAFOROU, London (GB); Jethro HERBERG, London (GB); Dominic HABGOOD-COOTE, London (GB)

(21) Appl. No.: 19/168,215

(22) PCT Filed: Mar. 22, 2024
(86) PCT No.: PCT/GB2024/050788

§ 371 Date: Sep. 23, 2025

(30) Foreign Application Priority Data
Mar. 23, 2023 (GB) 2304229.4

(51) Int. Cl. C12Q 1/6883; C12Q 1/689; C12Q 1/6893; C12Q 1/70

(52) U.S. Cl. CPC C12Q 1/6883; C12Q 1/689; C12Q 1/6893; C12Q 1/701; C12Q 2600/158

(57) ABSTRACT

The invention relates to gene signatures. In particular, the invention relates methods and kits for diagnosing a subject having a disease, by simultaneously discriminating between multiple infectious and inflammatory diseases using a gene signature. The invention also relates to the use of a gene signature as a diagnostic or prognostic biomarker for diseases.

### CROSS REFERENCE TO RELATED APPLICATIONS

This application is the U.S. National Stage Application under 35 U.S.C. § 371 of International Application No. PCT/GB2024/050788, filed Mar. 22, 2024, designating the U.S. and published in English as WO 2024/194656 A1 on Sep. 26, 2024, which claims the benefit of Great Britain Patent Application No. GB 2304229.4, filed Mar. 23, 2023, each of which is incorporated by reference herein in its entirety. Similarly, any and all applications for which a foreign or a domestic priority is claimed is/are identified in the Application Data Sheet filed herewith and is/are hereby incorporated by reference in their entirety under 37 C.F.R. § 1.57.

### SEQUENCE LISTING IN ELECTRONIC FORMAT

The present application includes an Electronic Sequence Listing as an XML file. The Electronic Sequence Listing is provided as a file entitled 126842PCT1.xml, created on Mar. 15, 2024, which is 147,332 bytes in size. The information in the Electronic Sequence Listing is incorporated herein by reference in its entirety.

### FIELD

The present invention relates to gene signatures, and particularly, although not exclusively, to methods and kits for diagnosing a subject having a disease, by simultaneously discriminating between multiple infectious and inflammatory diseases using a gene signature. The invention also relates to the use of a gene signature as a diagnostic or prognostic biomarker for diseases.

### DESCRIPTION OF RELATED ART

Infectious and inflammatory diseases are the most common causes of children seeking medical care in both hospital and community settings. It is a considerable challenge for clinical teams to identify and appropriately treat the small proportion of patients who have severe bacterial infection or inflammatory conditions, whilst avoiding over-treating the majority of patients who have self-limiting, usually viral, illness.

### SUMMARY

There is provided a method for diagnosing a subject having at least one disease, by simultaneously discriminating between at least three diseases and identifying the aetiology of the at least one disease that the subject is suffering from, the method comprising detecting, in a subject-derived RNA sample, the modulation in gene expression levels of a gene signature selected from the subject's transcriptome, to thereby diagnose a subject having at least one disease.

### BRIEF DESCRIPTION OF THE DRAWINGS

FIG. 1 shows a confusion matrix for gene expression microarray test set predictions. Performance of a 161-transcript signature in the 25% microarray test set over 18 specific disease classes (A) and over 6 broad disease classes (B). Confusion matrices show the numbers of each type of misclassification made where each sample is predicted to belong to the class with highest probability. Cost-weighting, point estimates for sensitivity and specificity for each prediction are shown on the right. E. coli: Escherichia coli, GAS: group A Streptococcus, GBS: group B Streptococcus, N. meningititis: Neisseria meningitidis, S. pneumoniae: Streptococcus pneumoniae, S. aureus: Staphylococcus aureus, HHV6: Human herpesvirus 6, RSV: respiratory syncytial virus, HSP: Henoch-Schönlein purpura, JIA: juvenile idiopathic arthritis, SLE: systemic lupus erythematosus, KD: Kawasaki disease.

FIG. 2 shows the microarray test set predictions of broad disease categories. Pairwise and one-versus-all discrimination of broad disease categories. Scatter plots and ROC curves are shown for pairs of disease categories (columns 1-6). Each scatter plot shows the predicted probabilities for patients with one of a pair condition; conditions for each scatter plot are given on the diagonal, above (x axis) and to the right (y axis) of each plot. ROC curves show the performance when distinguishing each pair of conditions; conditions for each ROC plot are given on the diagonal, below and to the left of each plot. Separation of the pair of diseases is performed using the predicted probabilities of both classes where the decision threshold is defined by varying the gradient of a line p(class1)=m*p(class2). The rightmost panels (columns 7-8) show the predicted probabilities for each class (left) and the one-vs-all ROC curve defined using only these probabilities to distinguish the class in a one-versus-all comparison. (*Confidence intervals not calculated due to lack of overlap).

FIG. 3 shows predicted probabilities for all classes shown on a per patient basis in the microarray test set. Predicted probabilities are shown for all patients in the microarray test set. Patients are grouped by the clinically assigned (true) diagnosis in the 6 major disease groups: (A) Bacterial diagnosis (B) Viral diagnosis (C) Inflammatory diagnosis (D) Tuberculosis diagnosis (E) Kawasaki Disease diagnosis (D) Malaria diagnosis. For each disease group, each patient corresponds to a row in the relevant different panel. For each patient (row) predicted probabilities for each sample sum to 1 (black) or can sum to more than 1 (grey). The bold boxed column corresponds to the true disease category. The labels at the top and bottom of the plot correspond to the clinically-assigned disease diagnosis.

FIG. 4 shows the performance of the 145-transcript panel in the validation cohort. Multi-class confusion matrices for disease prediction in the RNA-Seq validation set for specific disease classes (A); and for broad disease classes (B). Circle area corresponds to number of patients. Specificities and sensitivities for the detection of each class were derived from discrete class predictions. Cost-weighting and point estimates for sensitivity and specificity for each prediction are shown on the right. E. coli: Escherichia coli, GAS: group A Streptococcus, N. meningititis: Neisseria meningitidis, S. pneumoniae: Streptococcus pneumoniae, S. aureus: Staphylococcus aureus, RSV: respiratory syncytial virus, JIA: juvenile idiopathic arthritis, KD: Kawasaki Disease.

FIG. 5 shows the RNA-Seq validation set predictions of broad disease classes. Pairwise and one-versus-all discrimination of broad disease categories. Scatter plots and ROC curves are shown for pairs of disease categories (columns 1-6). Each scatter plot shows the predicted probabilities for patients with one of a pair condition; conditions for each scatter plot are given on the diagonal, above (x axis) and to the right (y axis) of each plot. ROC curves show the performance when distinguishing each pair of conditions; conditions for each ROC plot are given on the diagonal, below and to the left of each plot. Separation of the pair of diseases is performed using the predicted probabilities of both classes where the decision threshold is defined by varying the gradient of a line p(class1)=m*p(class2). The rightmost panels (columns 7-8) show the predicted probabilities for each class (left) and the one-vs-all ROC curve defined using only these probabilities to distinguish the class in a one-versus-all comparison.

FIG. 6 shows predictions of the broad disease model in the RNA-Seq cohort. Model coefficients for the 161 probes were fitted over the combined training and test sets from the microarray dataset, predictions in the RNA-Seq cohort of each broad disease category are based on these coefficients as independent linear models without applying the softmax function. Scatter plot axes are adjusted such that zero and one correspond to lowest and highest values respectively. ROC curves for pairwise comparisons are derived from the ratio of predictions.

FIG. 7 shows RNA-Seq validation set predictions for specific disease categories. Pairwise and one-versus-all discrimination of classes. Scatter plots show the predicted probabilities for the pair of classes above (X axis) and to the right (Y axis) in samples over samples in each pair of groups. ROC curves show the performance when distinguishing each pair of classes (below and to the left) when using the predicted probabilities of both classes where the decision threshold is defined by varying the gradient of a line p(class1)=m*p(class2). The rightmost panels show the predicted probabilities for each class (left) and the ROC curve defined using only these probabilities to distinguish the class in a one-versus-all comparison. (* confidence intervals could not be calculated due to lack of overlap.

FIG. 8 shows predicted probabilities for all classes shown on a per patient basis in the RNA-Seq dataset. Predicted probabilities are shown for all patients in the RNA-Seq dataset. Patients are grouped by the clinically assigned (true) diagnosis in the 6 major disease groups: (A) Bacterial diagnosis (B) Viral diagnosis (C) Inflammatory diagnosis (D) Tuberculosis diagnosis (E) Kawasaki Disease diagnosis (D) Malaria diagnosis. For each disease group, each patient corresponds to a row in the relevant different panel. For each patient (row) predicted probabilities for each sample sum to 1 (black) or can sum to more than 1 (grey). The bold boxed columns correspond to the true disease category. The labels at the top and bottom of the plot correspond to the clinically-assigned disease diagnosis.

FIG. 9 shows the comparison of the multi-class RNA signature to previously published signatures of infectious disease. ROC curves and 95% confidence intervals of specificity are shown for the multi-class signature and previously reported signatures for: tuberculosis (A), Kawasaki disease (B) and for distinguishing bacterial and viral infection (C, D, E). The comparison to the bacterial-viral signature is split by the formulation of the classification problem. (C) shows a bacterial versus viral comparison where for the multi-class classifier the ratio of predicted probabilities for bacterial and viral infection are used. (D) and (E) show the problem as a viral versus all and bacterial versus all respectively with each using the corresponding component of the multi-class signature.

FIG. 10 shows Table 1, which shows the genes identified in the gene signature according to the invention. The table also shows the selected microarray probes and their overlap in the RNAseq data. The probes highlighted in grey correspond to multiple potential genes. Probe IDs are given as illumina nuIDs.

FIG. 11 shows Table 13, which lists the genes of the gene signature and indicates whether they are up-regulated (+) or down-regulated (−) depending on the disease of the subject. This modulation of gene expression is comparing one disease to the rest of the diseases.

FIG. 12 shows Table 14, which lists the probes used to identify the genes of the gene signature according to the invention. Table 14 identifies the NCBI Accession Number of the probes, as well as their nucleotide sequence (with the corresponding SEQ ID No).

### DETAILED DESCRIPTION

Conventional diagnostic tests cannot simultaneously distinguish and accurately identify the multitude of potential aetiologies with sufficient speed and accuracy to inform initial treatment. Culture-based microbiological diagnosis is slow, and while molecular diagnostic techniques are faster, they are limited by the pathogens included in the panel and positive results may identify pathogens that are not the cause of the current illness, particularly for respiratory samples. Infection can involve either a single causative pathogen or the interaction of multiple organisms, limiting the utility of viral pathogen detection. Infections are frequently localised in inaccessible sites (such as the lungs) and, consequently pathogen detection from accessible sites, such as blood, urine or cerebrospinal fluid is frequently negative, even when severe infections are present.

For most inflammatory disorders, there is currently no single test to confirm or refute the diagnosis, and therefore patients with conditions, such as Kawasaki disease or juvenile idiopathic arthritis, are often not diagnosed until after a long period of hospitalisation, treatment for presumed infection and numerous investigations. As a result of the limitations of existing diagnostics, definitive final diagnoses are made for less than 50% of children attending an emergency department with fever, and in only half of children admitted to paediatric intensive care with suspected infection. Given this diagnostic uncertainty, many patients without bacterial infection are unnecessarily treated with broad-spectrum antibiotics to mitigate the risks of missing severe bacterial infection, contributing to the growing problem of antimicrobial drug resistance.

Gene expression microarrays and, more recently, RNA sequencing (RNA-Seq), have revealed an alternative approach, in which infectious or inflammatory diseases can be characterized by unique patterns of host gene expression in patients' blood, thereby bypassing the need for direct pathogen detection. There is a growing literature documenting that specific infectious and inflammatory diseases can be distinguished from conditions with similar presenting features using sparse transcriptional signatures in whole blood, including: discriminating between bacterial and viral infections, malaria, dengue virus, respiratory syncytial virus, rotavirus, tuberculosis (TB) and diagnosing inflammatory conditions such as Kawasaki disease (KD) and systemic lupus erythematosus (SLE).

However, a problem with such previous studies is that they use gene expression for diagnosis which focus on only very simplified binary distinctions, for example either one versus one (e.g. bacterial versus viral) or one versus all (e.g. tuberculosis versus other diseases). However, in clinical practice, there is a hierarchy of diagnostic categories, and many potential aetiologies must be considered and prioritised according to the risks posed by each. Furthermore, previous work is imprecise, as it can only inform the clinician whether an infection is bacterial, viral, or neither, or whether the patient has inflammatory disease, or not; it does not provide any specific information on which specific bacteria, virus or inflammatory disease is the cause of the illness.

There is, therefore, the need for a method of simultaneously discriminating multiple infectious and inflammatory diseases, and identifying the causative infectious agent, by a limited number of gene transcripts measured in patients' blood.

Accordingly, in a first aspect of the invention, there is provided a method for diagnosing a subject having at least one disease, by simultaneously discriminating between at least three diseases and identifying the aetiology of the at least one disease that the subject is suffering from, the method comprising detecting, in a subject-derived RNA sample, the modulation in gene expression levels of a gene signature selected from the subject's transcriptome, to thereby diagnose a subject having at least one disease.

Advantageously, as demonstrated in the examples, the inventors have surprisingly demonstrated that diverse infectious and inflammatory diseases can be simultaneously discriminated and identified by the expression levels of a single panel of genes in blood.

Advantageously, and preferably, the method of the invention enables a precise diagnosis, not just whether the disease is bacterial, or viral, or neither, but which specific bacteria, virus, or inflammatory disease is the cause of the disease.

Advantageously, and preferably, the method of the invention enables the assessment of many different possible diseases simultaneously, not just two to three disease categories.

Advantageously, and preferably, the method of the invention enables the detection of the co-occurrence of diseases, for example, co-infection with bacteria and virus(es).

Advantageously, and preferably, the disease categories which are simultaneously considered do not have to be mutually exclusive.

Advantageously, and preferably, the method of the invention assigns probabilities to different diseases, such that a clinician can consider not just the most likely one, but may also determine whether they need to account for other diseases which are less likely, but may have important implications for treatment, infection control and outcome.

The data described in the Examples and Figures show that diagnosis and classification of febrile illness can be achieved on a single blood sample and open the way to a new approach for diagnosis, particularly of children with fever.

The invention also provides a kit for identifying and simultaneously discriminating between multiple infectious and inflammatory diseases in a subject.

Hence, according to a second aspect of the invention, there is provided a diagnostic kit for diagnosing a subject having at least one disease, by simultaneously discriminating between at least three diseases and identifying the aetiology of the at least one disease that the subject is suffering from, the kit comprising means for detecting, in a subject-derived RNA sample, the modulation in gene expression levels of at least two genes selected from a gene signature in Table 1.

As described in the Examples, the inventors have demonstrated that the panel of genes listed in Table 1 can be used to discriminate between several types of disease and provide a diagnosis in a subject.

Accordingly, in a third aspect of the invention, there is provided the use of at least two genes selected from a gene signature in Table 1, as a diagnostic or prognostic biomarker for at least one disease.

The method of the first aspect can aid in the appropriate treatment of patients, for example where it is unclear if the patient is suffering from a bacterial infection, a viral infection, and/or an inflammatory disease. This has the advantage of ensuring rapid and appropriate treatment, and ensures that treatments are only prescribed when the subject genuinely has a disease which the treatment is intended to treat.

The rapid identification of causative pathogens would be useful for optimal treatment and choice of antibiotics, and clinical teams require a high degree of confidence in the broad disease category (i.e. viral, bacterial or inflammatory) to ensure potentially life-threatening conditions are not missed and to direct empiric treatment and appropriate subsequent investigations.

Thus, in one embodiment, the method according to the first aspect comprises a step of administering a therapeutic agent to the subject based on the results of the analysis of the gene signature.

Accordingly, in a fourth aspect of the invention, there is provided a method of treating a subject suffering from at least one disease, the method comprising:
(i) detecting, in a subject-derived RNA sample, the modulation in gene expression levels of a gene signature selected from the subject's transcriptome;
(ii) simultaneously discriminating between at least three diseases and identifying the aetiology of the at least one disease that the subject is suffering from; and
(ii) administering, or having administered, to the test subject, a therapeutic agent or putting the test subject on a specialised diet, wherein the therapeutic agent or the specialised diet prevents, reduces or delays progression of the at least one disease.

Advantageously, the methods, kit and use, according to the invention are able to assess, discriminate and identify between several different possible diseases simultaneously. Typically, the discrimination and identification can be achieved in a single step. Accordingly, in one preferred embodiment, the method comprises simultaneously discriminating between at least four diseases, at least five diseases, at least six diseases, at least seven diseases, at least eight diseases, at least nine diseases, or at least ten diseases.

Alternatively, in another preferred embodiment, the method comprises simultaneously discriminating between at least 12 diseases, at least 14 diseases, at least 16 diseases, at least 18 diseases, or at least 20 diseases. In another preferred embodiment, the method comprises simultaneously discriminating between at least 25 diseases, at least 30 diseases, at least 35 diseases, at least 40 diseases, at least 45 diseases, or at least 50 diseases. In another preferred embodiment, the method comprises simultaneously discriminating between at least 60 diseases, at least 70 diseases, at least 80 diseases, at least 90 diseases, or at least 100 diseases. In another preferred embodiment, the method comprises simultaneously discriminating between at least 120 diseases, at least 140 diseases, at least 160 diseases, at least 180 diseases, or at least 200 diseases.

Preferably, the at least one disease that the subject is suffering from, and the at least three diseases that the methods are able to discriminate between, are an infectious disease, an inflammatory disease, cancer, a metabolic disease, a degenerative disease, an endocrine disease and/or drug toxicity. The infectious disease may be a bacterial infection, a viral infection, or a parasitic infection. Accordingly, in a preferred embodiment, the methods of the invention comprise simultaneously discriminating between a bacterial infection, a viral infection, a parasitic infection, an inflammatory disease, cancer, a metabolic disease, a degenerative disease, an endocrine disease and/or drug toxicity.

Preferably, the methods of the invention comprise simultaneously discriminating between an infectious disease and an inflammatory disease. More preferably, the methods of the invention comprise simultaneously discriminating between a bacterial infection, a viral infection, a parasitic infection, and/or an inflammatory disease. In a most preferred embodiment, the method of the invention comprises simultaneously discriminating between a bacterial infection, a viral infection, a parasitic infection, and an inflammatory disease.

Advantageously, using the methods according to the invention, the diagnosis is precise because the aetiology of the disease that the subject is suffering from is identified, i.e. the specific bacteria, virus or inflammatory disease that is the cause of the illness. The aetiology of the disease refers to the cause or manner of the disease, for example, the specific bacteria or virus responsible for an infectious disease, or the specific type of inflammatory disease.

Accordingly, in an embodiment in which the subject is suffering from an infectious disease, the method comprises identifying the pathogen responsible for the infectious disease from which the subject is suffering from. Preferably, identifying the pathogen comprises naming the pathogen's genus, and more preferably, the pathogen's species.

In an embodiment in which the subject is suffering from an inflammatory disease, the method comprises identifying the specific type of inflammatory disease from which the subject is suffering from. Preferably, identifying the type of inflammatory disease comprises naming disease at the disease or syndrome level. The syndrome level refers to a recognisable complex of symptoms and physical findings which indicate a specific condition for which a direct cause is not necessarily understood. Accordingly, identifying the type of inflammatory disease at the syndrome level may comprise identifying a group of symptoms that indicate an inflammatory disease. The disease level refers to the causative agent or process responsible for the disease, which has clearly identifiable diagnostic features, disease progression and response to specific treatment. Accordingly, identifying the type of inflammatory disease at the disease level may comprise identifying the defining cause of the inflammatory disease.

In another embodiment in which the subject is suffering from cancer, a metabolic disease, a degenerative disease, an endocrine disease and/or drug toxicity, the method comprises identifying the specific type of these diseases from which the subject is suffering from, for example by naming the disease at the disease or syndrome level.

In one preferred embodiment, the bacterial infection is a Gram positive bacterial infection selected from the group consisting of: Corynebacterium diphtheriae, Clostridium botulinum, Clostridium difficile, Clostridium perfringens, Clostridium tetani, Enterococcus faecalis, Enterococcus faecium, Listeria monocytogenes, Staphylococcus aureus, Staphylococcus epidermidis, Staphylococcus saprophyticus, Group A Streptococcus, Group B streptococcus, Streptococcus agalactiae, Streptococcus pneumoniae, Streptococcus pyogenes, or acid fast bacteria such as Mycobacterium leprae, Mycobacterium tuberculosis, Mycobacterium ulcerans and Mycobacterium avium intercellularae, or a Gram negative bacterial infection selected from the group consisting of: Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Escherichia coli, Francisella tularensis, Haemophilus influenzae, Helicobacter pylori, Legionella pneumophila, Leptospira interrogans, Mycoplasma pneumonia, Neisseria gonorrhoeae, Neisseria meningitidis, Pseudomonas aeruginosa, Pseudomonas spp, Rickettsia rickettsii, Salmonella typhi, Salmonella typhimurium, Shigella sonnei, Treponema pallidum, Vibrio cholerae, Yersinia pestis, Kingella kingae, Stenotrophomonas and Klebsiella.

In one preferred embodiment, the bacterial infection is selected from the group consisting of: Mycobacterium tuberculosis, Staphylococcus aureus, Escherichia coli, Group A Streptococcus, Streptococcus pneumoniae, Group B streptococcus, and Neisseria meningitidis.

Preferably, the viral infection is selected from the group consisting of: influenza such as Influenza A, including but not limited to: H1N1, H2N2, H3N2, H5N1, H7N7, H1N2, H9N2, H7N2, H7N3, H10N7, Influenza B and Influenza C, Respiratory Syncytial Virus (RSV), rhinovirus, enterovirus, bocavirus, parainfluenza, adenovirus, metapneumovirus, herpes simplex virus, Chickenpox virus, Human papillomavirus, Hepatitis, Epstein-Barr virus, Varicella-zoster virus, Human cytomegalovirus, Human herpesvirus, human herpesvirus 6 (HHV6), type 8 BK virus, JC virus, Human coronaviruses, Smallpox, Parvovirus B19, Human astrovirus, Norwalk virus, coxsackievirus, poliovirus, Severe acute respiratory syndrome coronaviruses, yellow fever virus, dengue virus. West Nile virus, Rubella virus, Human immunodeficiency virus, Guanarito virus, Junin virus, Lassa virus, Machupo virus, Sabia virus, Crimean-Congo haemorrhagic fever virus, Ebola virus, Marburg virus, Measles virus, Mumps virus, Rabies virus and Rotavirus.

In one preferred embodiment, the viral infection is selected from the group consisting of: influenza, Respiratory Syncytial Virus (RSV), enterovirus, adenovirus, human herpesvirus 6 (HHV6), and rhinovirus.

Preferably, the parasitic infection is selected from the group including but not limited to: Malaria, African trypanosomiasis, babesiosis, Chagas disease, leishmaniasis, and toxoplasmosis.

In one preferred embodiment, the parasitic infection is malaria.

Preferably, the inflammatory disease is selected from the group including but not limited to: systemic lupus erythematosus (SLE), juvenile idiopathic arthritis (JIA), Henoch-Schönlein purpura (HSP), Kawasaki disease, rheumatoid arthritis, ulcerative colitis, Crohn's disease, chronic active hepatitis, celiac disease and vasculitis.

In one preferred embodiment, the inflammatory disease is selected from the group consisting of: Kawasaki disease, systemic lupus erythematosus (SLE), juvenile idiopathic arthritis (JIA), and Henoch-Schönlein purpura (HSP).

Advantageously, the methods, kit and use of the gene signature according to the claimed invention can detect the co-occurrence of diseases, such as co-infection with a bacteria and a virus(es). Accordingly, in a preferred embodiment, the at least three diseases that are simultaneously discriminated between are not mutually exclusive. For example, in a preferred embodiment, the at least three disease that are discriminated between are a bacterial infection, a viral infection, an inflammatory disease, malaria, tuberculosis and Kawasaki disease.

In one preferred embodiment, the method comprises diagnosing a subject having at least two diseases, and identifying the aetiology of the at least two diseases that the subject is suffering from. For example, in one embodiment, the method comprises diagnosing a subject having at least one bacterial infection and at least one viral infection, and identifying the aetiology of the at least one bacterial infection and the at least one viral infection that the subject is suffering from. In another embodiment, the method comprises diagnosing a subject having at least one bacterial infection and at least one inflammatory disease, and identifying the aetiology of the at least one bacterial infection and the at least one inflammatory disease that the subject is suffering from. In another embodiment, the method comprises diagnosing a subject having at least one viral infection and at least one inflammatory disease, and identifying the aetiology of the at least one viral infection and the at least one inflammatory disease that the subject is suffering from.

Alternatively, in another embodiment, the method comprises diagnosing a subject having at least three diseases, and identifying the aetiology of the at least three diseases that the subject is suffering from. For example, in one embodiment, the method comprises diagnosing a subject having at least one bacterial infection, at least one viral infection, and at least one inflammatory disease, identifying the aetiology of the at least one bacterial infection, the at least one viral infection, and the at least one inflammatory disease that the subject is suffering from.

The subject may be a vertebrate, mammal, or domestic animal. Most preferably, however, the subject is a human being. The subject may be a male or female.

The subject may be a child or adult. The subject may be under the age of 20, under the age of 15, under the age of 10, or under the age of 5. Alternatively, the age of the subject may be at least 20, 25, 30, 35, 40, 45 or 50. The age of the subject may be at least 55, 60, 65, 70, 75, 80, 85, 90, 95 or 100.

Preferably, the subject is a febrile child.

The term “gene signature” as used herein, refers to a set of genes which when tested together are able to detect and/or discriminate the relevant clinical status. Hence, a gene signature represents a minimal set of genes which have sufficient discriminatory power to identify and discriminate the specific type of infectious or inflammatory disease a subject is suffering from.

In one preferred embodiment, the method comprises detecting the modulation in gene expression levels of at least 2 genes, at least 5 genes, at least 10 genes, at least 15 genes, at least 20 genes, at least 25 genes, at least 30 genes, at least 35 genes, at least 40 genes, at least 45 genes, or at least 50 genes, of a gene signature. More preferably, the method comprises detecting the modulation in gene expression levels of at least 55 genes, at least 60 genes, at least 65 genes, at least 70 genes, at least 75 genes, at least 80 genes, at least 85 genes, at least 90 genes, at least 95 genes, or at least 100 genes, of a gene signature. Even more preferably, the method comprises detecting the modulation in gene expression levels of at least 105 genes, at least 110 genes, at least 115 genes, at least 120 genes, at least 125 genes, at least 130 genes, at least 135 genes, at least 140 genes, at least 145 genes, at least 150 genes, at least 155 genes, or at least 160 genes, of a gene signature.

In particular, the inventors have demonstrated that the panel of genes listed in Table 1 can be used to discriminate between several types of disease and provide a diagnosis in a subject.

Accordingly, in one preferred embodiment, the gene signature comprises a list of genes defined in Table 1. In one embodiment, the method comprises detecting the modulation in gene expression levels of at least 2 genes, at least 5 genes, at least 10 genes, at least 15 genes, at least 20 genes, at least 25 genes, at least 30 genes, at least 35 genes, at least 40 genes, at least 45 genes, or at least 50 genes, of a gene signature in Table 1. More preferably, the method comprises detecting the modulation in gene expression levels of at least 55 genes, at least 60 genes, at least 65 genes, at least 70 genes, at least 75 genes, at least 80 genes, at least 85 genes, at least 90 genes, at least 95 genes, or at least 100 genes, of a gene signature in Table 1. Even more preferably, the method comprises detecting the modulation in gene expression levels of at least 105 genes, at least 110 genes, at least 115 genes, at least 120 genes, at least 125 genes, at least 130 genes, at least 135 genes, at least 140 genes, at least 145 genes, at least 150 genes, at least 155 genes, or at least 160 genes, of a gene signature show in Table 1. In a preferred embodiment, the method comprises detecting the modulation in gene expression levels of 155 genes of a gene signature in Table 1. In a most preferred embodiment, the method comprises detecting the modulation in gene expression levels of all 161 genes of a gene signature in Table 1.

The inventors discovered that ten of the genes in the gene signature did not have sufficient read counts in the RNA-Seq dataset for reliable quantification, and thus were only detected at a moderate level (“Low expression in RNA seq” in Table 1). However, it will be appreciated that this level of detection occurred when using just one platform, and many other platforms that are more sensitive, for example, may be able to detect these ten genes in an RNA-Seq dataset. In one embodiment, the gene signature comprises or consists of the genes in Table 1 labelled with “TRUE” in RNA-Seq data. Preferably, the gene signature does not comprise the genes labelled with “Low expression in RNA seq” in RNA-Seq data in Table 1.

In one preferred embodiment, when the at least one disease is influenza, the method comprises detecting the modulation in gene expression levels of at least two genes selected from the group consisting of: HESX1, IL4I1, AXL, IER5, RNASE1, NTN3, PID1, TEF, and C9orf21. Alternatively and/or additionally, when the at least one disease is influenza, the method comprises detecting the modulation in gene expression levels with a probe comprising a nucleotide sequence as set out in SEQ ID No: 17.

In one preferred embodiment, when the at least one disease is respiratory syncytial virus (RSV), the method comprises detecting the modulation in gene expression levels of at least two genes selected from the group consisting of: HIST2H2BE, FLJ10213, PAK2, IFI6, ATP6V1G1, IFI27, EBI3, HNRPM, NOV, and PINX1.

In one preferred embodiment, when the at least one disease is adenovirus, the method comprises detecting the modulation in gene expression levels of at least two genes selected from the group consisting of: GLDC, BFSP2, IFI27, NOV, COQ10A, CHRM4, LOC648526, HSP90AA1, CLC, and MXRA7.

In one preferred embodiment, when the at least one disease is Kawasaki disease, the method comprises detecting the modulation in gene expression levels of at least two genes selected from the group consisting of: CCL23, APOL3, CETP, NTN3, ITGAX, HIST1H2BK, PRR5, MT1F, PLA2G7, and HIST1H3H.

In one preferred embodiment, when the at least one disease is group A Streptococcus, the method comprises detecting the modulation in gene expression levels of at least two genes selected from the group consisting of: C9orf21, PARP8, GLDC, LOC644162, GIMAP4, MID1, IL8, LOC641705, HOXC10, and ITGAX.

In one preferred embodiment, when the at least one disease is juvenile idiopathic arthritis, the method comprises detecting the modulation in gene expression levels of at least two genes selected from the group consisting of: KIAA1600, NOV, PID1, NDRG2, LOC389816, HOXC10, CD24, EGLN2, IFI27, and BHLHB2.

In one preferred embodiment, when the at least one disease is systemic lupus erythematosus, the method comprises detecting the modulation in gene expression levels of at least two genes selected from the group consisting of: ZCCHC2, REC8, IFI27, NOV, IFI44L, IL8, TYSND1, TNRC6A, GAS6, and PIK3CG.

In one preferred embodiment, when the at least one disease is Human herpesvirus 6, the method comprises detecting the modulation in gene expression levels of at least two genes selected from the group consisting of: PARP8, TMED4, AC004080.2, MASTL, MXRA7, POLE4, HSP90AA1, PTPRM, MGC42367, and PRR5.

In one preferred embodiment, when the at least one disease is enterovirus, the method comprises detecting the modulation in gene expression levels of at least two genes selected from the group consisting of: RBM3, NPEPL1, GIMAP4, CDKN1C, RHOQ, TNFRSF21, IFI6, RTN1, LOC641705, and ITGAX.

In one preferred embodiment, when the at least one disease is rhinovirus, the method comprises detecting the modulation in gene expression levels of at least two genes selected from the group consisting of: LOC652185, SEPN1, EBF2, ARHGEF9, CTAGE6, S100A9, C18orf54, HLA-A, TNFRSF21, and MMP9.

In one preferred embodiment, when the at least one disease is Escherichia coli, the method comprises detecting the modulation in gene expression levels of at least two genes selected from the group consisting of: BID, REC8, HIST1H3F, LHPP, CAV2, NPEPL1, IGLL1, PLA2G7, AMFR, and S100A9.

In one preferred embodiment, when the at least one disease is Staphylococcus aureus, the method comprises detecting the modulation in gene expression levels of at least two genes selected from the group consisting of: HOXA9, MS4A3, ATP6V1G1, DEM1, TEF, TXNRD2, BMF, SEPN1, PINX1, and IFI27.

In one preferred embodiment, when the at least one disease is tuberculosis, the method comprises detecting the modulation in gene expression levels of at least two genes selected from the group consisting of: ARAP2, LOC644739, PRDM1, SNX6, RAP2C-AS1, TYSND1, PCGF6, EGLN2, KCTD12. Alternatively and/or additionally, when the at least one disease is tuberculosis, the method comprises detecting the modulation in gene expression levels with a probe comprising a nucleotide sequence as set out in SEQ ID No: 17.

In one preferred embodiment, when the at least one disease is malaria, the method comprises detecting the modulation in gene expression levels of at least two genes selected from the group consisting of: LOC650850, PDCD1, EBI3, TOMM70A, HSP90AA1, KIR3DL2, CTLA4, SAP18, MMP9, and VPS52.

In one preferred embodiment, when the at least one disease is Streptococcus pneumoniae, the method comprises detecting the modulation in gene expression levels of at least two genes selected from the group consisting of: RNASE3, COQ10A, DEM1, BRD2, VCX, CD24, HEMK1, TYSND1, CXCR6, and DEF6.

In one preferred embodiment, when the at least one disease is group B Streptococcus, the method comprises detecting the modulation in gene expression levels of at least two genes selected from the group consisting of: VCX, ATP2C2, CYTSA, EXT2, MT1F, ARHGEF9, RHOQ, IGLL1, HLA-A, and P2RX7.

In one preferred embodiment, when the at least one disease is Neisseria meningitidis, the method comprises detecting the modulation in gene expression levels of at least two genes selected from the group consisting of: CMTM1, RNASE1, MMD, AMACR, LOC644532, HEMK1, MTL5, C1QC, and CDKN1C. Alternatively and/or additionally, when the at least one disease is Neisseria meningitidis, the method comprises detecting the modulation in gene expression levels with a probe comprising a nucleotide sequence as set out in SEQ ID No: 161.

In one preferred embodiment, when the at least one disease is Henoch-Schönlein purpura, the method comprises detecting the modulation in gene expression levels of at least two genes selected from the group consisting of: C9orf117, APOL3, EPSTI1, SAP18, PCGF6, CYDL2, LOC648000, PID1, CHRM4, and CTAGE6.

Gene expression as referred to herein is the process by which information from a gene is used in the synthesis of a functional gene product. Gene expression can be determined by the abundance of the corresponding RNA (e.g., mRNA, noncoding RNA (ncRNA), miRNA, tRNA, rRNA, snoRNA, siRNA and piRNA). Measuring gene expression at the mRNA level may include measuring levels of cDNA corresponding to mRNA.

Accordingly, in a preferred embodiment, RNA from the sample is analysed. Preferably, the method comprises analysing mRNA, ncRNA, miRNA, tRNA, rRNA, snoRNA, siRNA or piRNA.

The modulation in gene expression levels as described herein may be the up-regulation or down-regulation of a gene or genes. Up-regulated gene expression refers to a gene transcript which is expressed at higher levels in a diseased or infected patient sample relative to, for example, a control sample free from a relevant disease or infection, or in a sample with latent disease or infection or a different stage of the disease or infection, as appropriate. Down-regulated gene expression refers to a gene transcript which is expressed at lower levels in a diseased or infected patient sample relative to, for example, a control sample free from a relevant disease or infection or in a sample with latent disease or infection or a different stage of the disease or infection.

In one preferred embodiment, one or more of the following genes are up-regulated in a subject having influenza: HESX1, IL4I1, AXL, IER5, RNASE1, TEF and C9orf21. In one preferred embodiment, one or more of the following genes are down-regulated in a subject having influenza: NTN3 and PID1. Preferably, these genes are up-regulated or down-regulated when compared to diseases other than influenza.

In one preferred embodiment, one or more of the following genes are up-regulated in a subject having respiratory syncytial virus (RSV): IFI6, ATP6V1G1, IFI27, and HNRPM. In one preferred embodiment, one or more of the following genes are down-regulated in a subject having respiratory syncytial virus (RSV): HIST2H2BE, FLJ10213, PAK2, EBI3, NOV, and PINX1. Preferably, these genes are up-regulated or down-regulated when compared to diseases other than respiratory syncytial virus (RSV).

In one preferred embodiment, one or more of the following genes are up-regulated in a subject having adenovirus: GLDC, BFSP2, IFI27, COQ10A, CHRM4, HSP90AA1, and MXRA7. In one preferred embodiment, one or more of the following genes are down-regulated in a subject having adenovirus: NOV, LOC648526, and CLC. Preferably, these genes are up-regulated or down-regulated when compared to diseases other than adenovirus.

In one preferred embodiment, one or more of the following genes are up-regulated in a subject having Kawasaki disease: CCL23, CETP, NTN3, ITGAX, HIST1H2BK, and HIST1H3H. In one preferred embodiment, one or more of the following genes are down-regulated in a subject having Kawasaki disease: APOL3, PRR5, MT1F, and PLA2G7. Preferably, these genes are up-regulated or down-regulated when compared to diseases other than Kawasaki disease.

In one preferred embodiment, one or more of the following genes are up-regulated in a subject having group A Streptococcus: GLDC and ITGAX. In one preferred embodiment, one or more of the following genes are down-regulated in a subject having group A Streptococcus: C9orf21, PARP8, LOC644162, GIMAP4, MID1, IL8, LOC641705, and HOXC10. Preferably, these genes are up-regulated or down-regulated when compared to diseases other than group A Streptococcus.

In one preferred embodiment, one or more of the following genes are up-regulated in a subject having juvenile idiopathic arthritis: NOV, PID1, NDRG2, LOC389816, HOXC10, EGLN2, and BHLHB2. In one preferred embodiment, one or more of the following genes are down-regulated in a subject having juvenile idiopathic arthritis: KIAA1600, CD24, and IFI27. Preferably, these genes are up-regulated or down-regulated when compared to diseases other than juvenile idiopathic arthritis.

In one preferred embodiment, one or more of the following genes are up-regulated in a subject having systemic lupus erythematosus: ZCCHC2, REC8, IFI27, NOV, IL8, IFI44L, TYSND1, and TNRC6A. In one preferred embodiment, one or more of the following genes are down-regulated in a subject having systemic lupus erythematosus: GAS6 and PIK3CG. Preferably, these genes are up-regulated or down-regulated when compared to diseases other than systemic lupus erythematosus.

In one preferred embodiment, one or more of the following genes are up-regulated in a subject having Human herpesvirus 6: PARP8, TMED4, AC004080.2, MASTL, MXRA7, POLE4, HSP90AA1, and PRR5. In one preferred embodiment, one or more of the following genes are down-regulated in a subject having Human herpesvirus 6: PTPRM and MGC42367. Preferably, these genes are up-regulated or down-regulated when compared to diseases other than Human herpesvirus 6.

In one preferred embodiment, one or more of the following genes are up-regulated in a subject having enterovirus: GIMAP4, CDKN1C, TNFRSF21, and IFI6. In one preferred embodiment, one or more of the following genes are down-regulated in a subject having enterovirus: RBM3, NPEPL1, RHOQ, RTN1, LOC641705, and ITGAX. Preferably, these genes are up-regulated or down-regulated when compared to diseases other than enterovirus.

In one preferred embodiment, one or more of the following genes are up-regulated in a subject having rhinovirus: LOC652185, ARHGEF9, C18orf54, EBF2, and TNFRSF21. In one preferred embodiment, one or more of the following genes are down-regulated in a subject having rhinovirus: SEPN1, CTAGE6, S100A9, HLA-A, and MMP9. Preferably, these genes are up-regulated or down-regulated when compared to diseases other than rhinovirus.

In one preferred embodiment, one or more of the following genes are up-regulated in a subject having Escherichia coli: BID, CAV2, PLA2G7, and S100A9. In one preferred embodiment, one or more of the following genes are down-regulated in a subject having Escherichia coli: REC8, HIST1H3F, LHPP, NPEPL1, IGLL1, and AMFR. Preferably, these genes are up-regulated or down-regulated when compared to diseases other than Escherichia coli.

In one preferred embodiment, one or more of the following genes are up-regulated in a subject having Staphylococcus aureus: HOXA9, MS4A3, TEF, TXNRD2, BMF, and SEPN1. In one preferred embodiment, one or more of the following genes are down-regulated in a subject having Staphylococcus aureus: ATP6V1G1, DEM1, PINX1, and IFI27. Preferably, these genes are up-regulated or down-regulated when compared to diseases other than Staphylococcus aureus.

In one preferred embodiment, one or more of the following genes are up-regulated in a subject having tuberculosis: ARAP2, LOC644739, PRDM1, PCGF6, RAP2C-AS1, and KCTD12. In one preferred embodiment, one or more of the following genes are down-regulated in a subject having tuberculosis: SNX6, TYSND1, and EGLN2. Preferably, these genes are up-regulated or down-regulated when compared to diseases other than tuberculosis.

In one preferred embodiment, one or more of the following genes are up-regulated in a subject having malaria: LOC650850, PDCD1, EBI3, TOMM70A, HSP90AA1, KIR3DL2, CTLA4, SAP18, MMP9, and VPS52. Preferably, these genes are up-regulated when compared to diseases other than malaria.

In one preferred embodiment, one or more of the following genes are up-regulated in a subject having Streptococcus pneumoniae: RNASE3, VCX, and CD24. In one preferred embodiment, one or more of the following genes are down-regulated in a subject having Streptococcus pneumoniae: COQ10A, DEM1, BRD2, HEMK1, TYSND1, CXCR6, and DEF6. Preferably, these genes are up-regulated or down-regulated when compared to diseases other than Streptococcus pneumoniae.

In one preferred embodiment, one or more of the following genes are up-regulated in a subject having group B Streptococcus: VCX, ATP2C2, and CYTSA. In one preferred embodiment, one or more of the following genes are down-regulated in a subject having group B Streptococcus: EXT2, MT1F, ARHGEF9, RHOQ, IGLL1, HLA-A, and P2RX7. Preferably, these genes are up-regulated or down-regulated when compared to diseases other than group B Streptococcus.

In one preferred embodiment, one or more of the following genes are up-regulated in a subject having Neisseria meningitidis: CMTM1, RNASE1, AMACR, LOC644532, HEMK1, MTL5, C1QC, and CDKN1C. In one preferred embodiment, the gene MMD is down-regulated in a subject having Neisseria meningitidis. Preferably, these genes are up-regulated or down-regulated when compared to diseases other than Neisseria meningitidis.

In one preferred embodiment, one or more of the following genes are up-regulated in a subject having Henoch-Schönlein purpura: PCGF6, LOC648000, PID1, and CHRM4. In one preferred embodiment, one or more of the following genes are down-regulated in a subject having Henoch-Schönlein purpura: C9orf117, APOL3, EPSTI1, SAP18, CYDL2, and CTAGE6. Preferably, these genes are up-regulated or down-regulated when compared to diseases other than Henoch-Schönlein purpura.

Alternatively, in another embodiment, one or more of the genes listed in Table 13 may be up-regulated (+) or down-regulated (−) in a subject having a particular disease, as shown in Table 13.

Methods of detecting modulation in gene expression levels will be well-known to those in the art. For example, the detection of modulation in gene expression levels may be measured using a microarray, DNA or RNA arrays, RNA-seq, or PCR, such as RT-PCR. In one embodiment, the PCR may be multiplex PCR.

As such, the means for detecting modulation in gene expression in the kit according to the second aspect, may include oligonucleotides that specifically hybridise to RNA of the genes in Table 1. Such oligonucleotides can be used as PCR primers in RT-PCR reactions, or hybridization probes. The oligonucleotides in the kit may be labelled with any suitable detection marker including but not limited to, radioactive isotopes, fluorophores, biotin, enzymes (e.g., alkaline phosphatase), enzyme substrates, ligands and antibodies.

In one embodiment, the oligonucleotides (PCR primers or hybridization probes) comprise a nucleotide sequence as set out in any one of SEQ ID Nos: 1 to 161.

In a fifth aspect, there is provided a gene chip comprising probes for detecting the modulation in gene expression levels of at least two genes of a gene signature in Table 1.

In one preferred embodiment, the gene chip comprises probes for detecting the modulation in gene expression levels of at least 5 genes, at least 10 genes, at least 15 genes, at least 20 genes, at least 25 genes, at least 30 genes, at least 35 genes, at least 40 genes, at least 45 genes, or at least 50 genes, of a gene signature in Table 1. More preferably, the gene chip comprises probes for detecting the modulation in gene expression levels of at least 55 genes, at least 60 genes, at least 65 genes, at least 70 genes, at least 75 genes, at least 80 genes, at least 85 genes, at least 90 genes, at least 95 genes, or at least 100 genes, of a gene signature in Table 1. Even more preferably, the gene chip comprises probes for detecting the modulation in gene expression levels of at least 105 genes, at least 110 genes, at least 115 genes, at least 120 genes, at least 125 genes, at least 130 genes, at least 135 genes, at least 140 genes, at least 145 genes, at least 150 genes, at least 155 genes, or at least 160 genes, of a gene signature show in Table 1. In a preferred embodiment, the gene chip comprises probes for detecting the modulation in gene expression levels of 155 genes of a gene signature in Table 1. In a most preferred embodiment, the gene chip comprises probes for detecting the modulation in gene expression levels of all 161 genes of a gene signature in Table 1.

Preferably, the gene chip does not contain probes for any other genes.

In one embodiment, the probes of the gene chip comprise a nucleotide sequence as set out in any one of SEQ ID Nos: 1 to 161.

In one embodiment the gene chip is a fluorescent gene chip, i.e. the readout is fluorescence. Fluorescence as employed herein refers to the emission of light by a substance that has absorbed light or other electromagnetic radiation.

In another embodiment, the gene chip is a colorimetric gene chip, for example colorimetric gene chip uses microarray technology wherein avidin is used to attach enzymes such as peroxidase or other chromogenic substrates to the biotin probe currently used to attach fluorescent markers to DNA. The present disclosure extends to a microarray chip adapted to be read by colorimetric analysis.

The methods may be carried out in vivo, in vitro or ex vivo. Preferably, however, the method is carried out in vitro.

The kit of the second aspect may comprise sample extraction means for obtaining the sample from the test subject. The sample extraction means may comprise a needle or syringe or the like. The kit may comprise a sample collection container for receiving the extracted sample, which may be liquid, gaseous or semi-solid.

The sample is preferably a biological bodily sample taken from the test subject. Detecting the modulation of gene expression levels is therefore preferably carried out in vitro. The sample may comprise tissue, blood, plasma, serum, spinal fluid, urine, sweat, saliva, sputum, tears, breast aspirate, prostate fluid, seminal fluid, vaginal fluid, stool, cervical scraping, amniotic fluid, intraocular fluid, mucous, moisture in breath, animal tissue, cell lysates, tumour tissue, hair, skin, buccal scrapings, nails, bone marrow, cartilage, prions, bone powder, ear wax, or combinations thereof. The sample may be a biopsy. Preferably, the sample is blood. Alternatively, the sample may be an ex vivo sample.

It will also be appreciated that “fresh” bodily samples may be analysed immediately after they have been taken from a subject. Alternatively, the samples may be frozen and stored. Preferably, an appropriate medium is added to the sample to protect the RNA from degradation. The sample may then be de-frosted and analysed at a later date.

The method and/or kit according to the invention may comprise the use of a positive control and/or a negative control against which the modulation in gene expression levels may be compared. For example, a negative control sample is a sample taken from a subject who does not have the at least one disease that the subject is suffering from. A positive control sample is a sample taken from a subject who does have the at least one disease that the subject is suffering from.

In another embodiment, the method and/or kit according to the invention may comprise the use of a reference gene (or housekeeping gene) against which the modulation in gene expression levels may be compared. A reference gene may be one which is invariant in the diseases of interest, i.e. it is transcribed at a relatively constant level. Examples of references genes include ACTB, B2M, GAPDH, HPRT, PGK1, RPS18, TBP, UBC.

Accordingly, in one embodiment, the method further comprises comparing the gene expression levels of the gene signature with the gene expression levels of the gene signature of a positive and/or negative control. The kit preferably comprises a positive control sample and/or a negative control sample. In one embodiment, an increase in expression levels of a gene compared to the negative control suggests that the subject does have the at least one disease. In another embodiment, a decrease in expression levels of a gene compared to the negative control suggests that the subject does have the at least one disease.

It is advantageous if the method according to the invention can assign probabilities to different diseases, such that a clinician can consider not just the most likely one (i.e. the one which would be assigned as the cause of the disease), but also to determine whether their clinical management needs to take account of other diseases, which are less likely but may have important implications for treatment, infection control and outcome.

Accordingly, in one embodiment, the method further comprises determining the likelihood that the subject suffers from the at least one disease.

For example, it will be appreciated that if a subject has a gene signature which has considerably modulated gene expression levels compared to a control sample free from a relevant disease or infection, or in a sample with latent disease or infection or a different stage of the disease or infection, then the subject would be more likely to suffer from the at least one disease. Alternatively, if a subject has a gene signature which has only slightly modulated gene expression levels compared to a control sample free from a relevant disease or infection, or in a sample with latent disease or infection or a different stage of the disease or infection, then the subject would be less likely to suffer from the at least one disease.

The skilled technician will appreciate how to analyse the gene expression levels of the gene signature in a statistically significant number of control samples, and the gene expression levels of the gene signature in the test subject, and then use these respective figures to determine whether the test subject has a statistically significant modulation in gene expression levels of the gene signature, and therefore infer the likelihood that the subject is suffering from the at least one disease.

In one preferred embodiment, the method comprises the use of a mathematical model, preferably a multinomial logistic regression model. The model takes the gene expression measurements for each sample or subject and makes a prediction in the form of a probability for each disease. At the time of making predictions, there is no statistical testing involved, the data is put into a model, function, or equation to get a prediction. The probabilities are predicted on the basis of gene expression. However, in another embodiment, the probabilities may be predicted on the basis of the population or context within which the test is applied, where the prior probabilities of different diseases will vary between each other and from the discovery cohort.

In an embodiment in which the at least one disease is a bacterial infection, preferably the method comprises administering an anti-bacterial agent, such as an antibiotic. Suitable anti-bacterial agents may be selected from the group including but not limited to: ceftobiprole, ceflaroline, clindamycin, dalbavancin, daptomycin, linezolid, oritavancin, tedizolid, telavancin, tigecycline, vancomycin, aminoglycosides, carbapenems, ceftazidime, ceftobiprole, fluoroquinolines, piperacillin/tazobactam, ticarcillin/clavulanic acid, streptogramins, such as amikacin, gentamicin, kanamycin, netilmicin, tobramycin, paromomycin, streptomycin, geldanamycin, herbimycin, rifaximin, loracarbef, ertapenem, doripenem, imipenem/cilastatin, meropenem, cefadroxil, cefazolin, cefalotin/cefalothin, cefalexin, cefaclor, k cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, dalbavancin, oritavancin, clindamycin, linomycin, daptomycin, azithromycin, clarithromycin, dirithromycin, erythromycin, roxithromycin, troleandomycin, telithromycin, spiramycin, aztreonam, furazilidone, linezolid, posizolid, radezolid, torezolid, amoxicillin, ampicillin, azlocillin, carbenicillin, cloxacillin, dicloxacillin, flucloxacillin, mezlocillin, nafcillin, oxacillin, penicillin G, penicillin V, piperacillin, temocillin, ticarcillin, amoxicillin/clavulanate, ampicillin/sulbactam, piperacillin/tazobactam, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfasalazine, sulfisoxazole, trimethoprim-sulfamethoxazole, sulfonamidochrysoidine, demeclocycline, doxycycline, minocycline, oxytetracycline, tetracycline, clofazimine, dapsone, capremycin, cycloserine, ethambutol, ethionamide, isoniazid, pyrazinamide, rifampicin, rifapentine, streptomycin, chloramphenicol, fosfomycin, fusidic acid, metronidazole, mupirocin, platensimycin, quinupristin/dalfopristin, thiamphenicol, tigecycline, tinidazole and trimethoprim.

In an embodiment in which the at least one disease is a viral infection, preferably the method comprises administering an anti-viral agent, an immunomodulatory agent, and/or supportive care where the viral infection is suspected to be self-limiting. Suitable anti-viral agents may be selected from the group including but not limited to: Aciclovir, valaciclovir, ganciclovir, valganciclovir, oseltamivir, zanamivir, amantadine, rimantadine, cidofovir, brincidofovir, famciclovir, foscarnet, molnupiravir, nirmatrelvir, remdesivir, sotrovimab, entecavir, interferon alfa, tenofovir, adefovir, dipivoxil, lamivudine, sofosbuvir, ribavirin, and immunoglobulin.

In an embodiment in which the at least one disease is a parasitic infection, preferably the method comprises administering an anti-parasitic agent. Suitable anti-parasitic agents may be selected from the group including but not limited to: Praziquantel, Mebendazole, Albendazole, Metronidazole, Ivermectin, Nitazoxanide, Suramin, Amphotericin B, Pentamidine, Melarsoprol, Eflornithine, Nifurtimox, Atovaquone, Azithromycin, Clindamycin, Quinine, Doxycycline, Benznidazole, Miltefosine, Paromomycin, Sodium stibogluconate, meglumine antimoniate, Chloroquine, Artesunate, Artemether, Mefloquine, Proguanil, Primaquine, Lumefantrine, Piperaquine, Pyronaridine, Sulfadiazine, Pyrimethamine, and Spiramycin.

In an embodiment in which the at least one disease is an inflammatory disease, preferably the method comprises administering an agent including but not limited to: Ibuprofen, naproxen, aspirin, hydroxychloroquine, colchicine, corticosteroids, methotrexate, sulfasalazine, adalimumab, infliximab, etanercept, anakinra, canakinumab, tocilizumab, sarilumab, baricitinib, tofacitinib, imatinib, upadacitinib, filgotinib, mesalamine, rituximab, belimumab, mycophenolate mofetil, cyclophosphamide, abatacept, ustekinumab, vedolizumab, natalizumab, plasma exchange, and immunoglobulin.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with any of the sequence identified herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from any of the sequences described herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

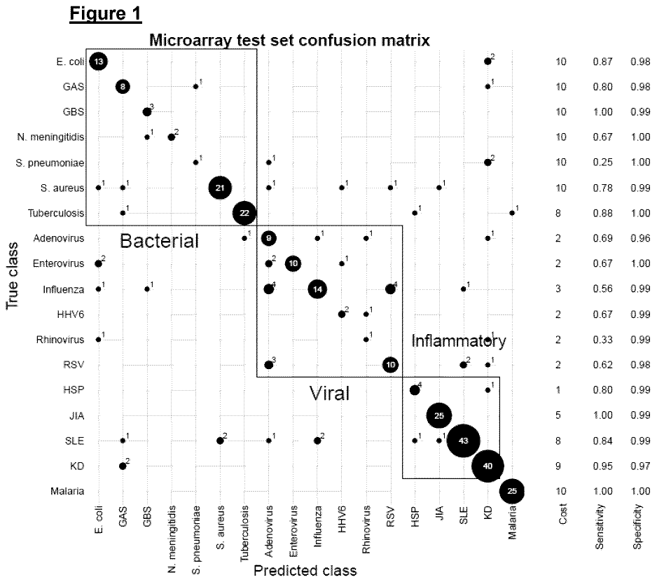

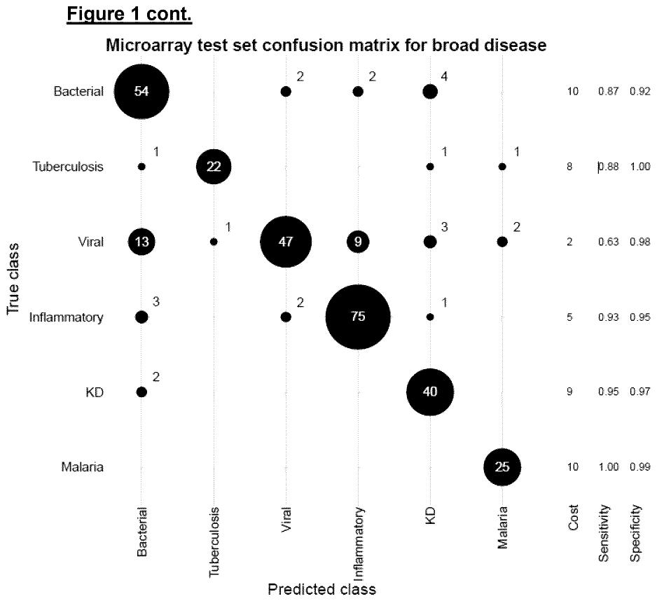

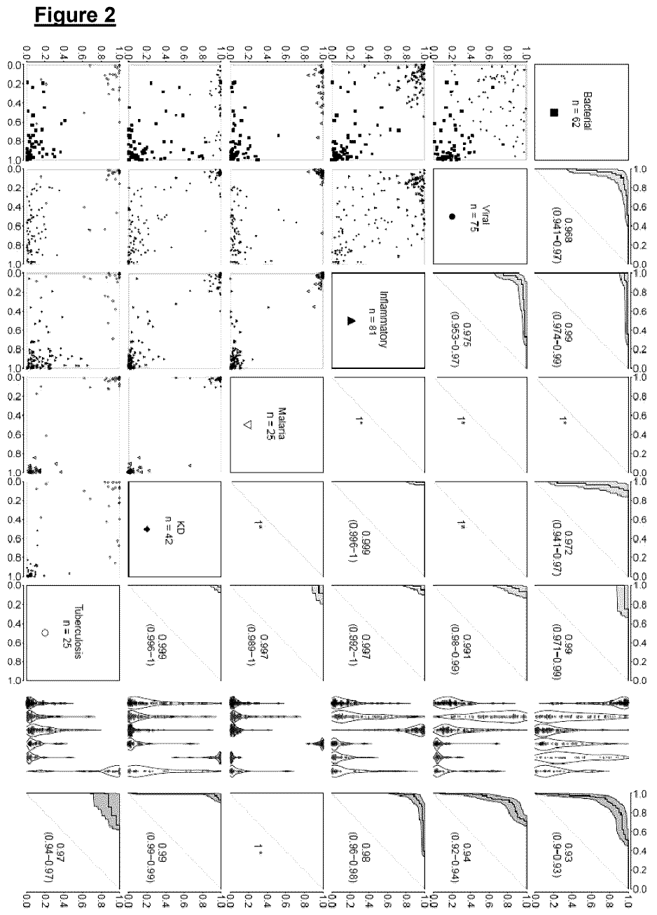

FIG. 1 shows a confusion matrix for gene expression microarray test set predictions. Performance of a 161-transcript signature in the 25% microarray test set over 18 specific disease classes (A) and over 6 broad disease classes (B). Confusion matrices show the numbers of each type of misclassification made where each sample is predicted to belong to the class with highest probability. Cost-weighting, point estimates for sensitivity and specificity for each prediction are shown on the right. E. coli: Escherichia coli, GAS: group A Streptococcus, GBS: group B Streptococcus, N. meningititis: Neisseria meningitidis, S. pneumoniae: Streptococcus pneumoniae, S. aureus: Staphylococcus aureus, HHV6: Human herpesvirus 6, RSV: respiratory syncytial virus, HSP: Henoch-Schönlein purpura, JIA: juvenile idiopathic arthritis, SLE: systemic lupus erythematosus, KD: Kawasaki disease.

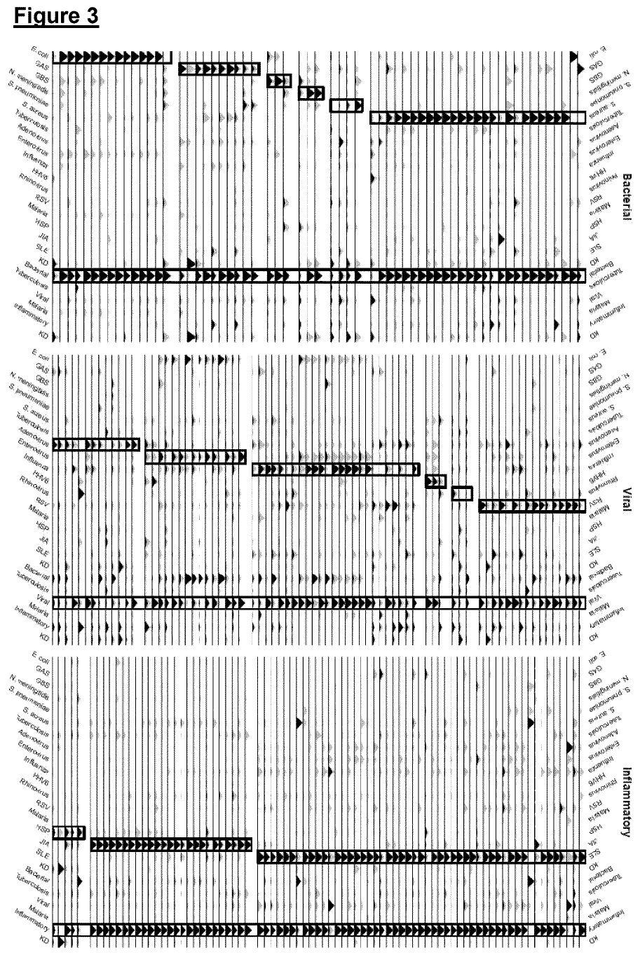

FIG. 2 shows the microarray test set predictions of broad disease categories. Pairwise and one-versus-all discrimination of broad disease categories. Scatter plots and ROC curves are shown for pairs of disease categories (columns 1-6). Each scatter plot shows the predicted probabilities for patients with one of a pair condition; conditions for each scatter plot are given on the diagonal, above (x axis) and to the right (y axis) of each plot. ROC curves show the performance when distinguishing each pair of conditions; conditions for each ROC plot are given on the diagonal, below and to the left of each plot. Separation of the pair of diseases is performed using the predicted probabilities of both classes where the decision threshold is defined by varying the gradient of a line p(class1)=m*p(class2). The rightmost panels (columns 7-8) show the predicted probabilities for each class (left) and the one-vs-all ROC curve defined using only these probabilities to distinguish the class in a one-versus-all comparison. (*Confidence intervals not calculated due to lack of overlap).

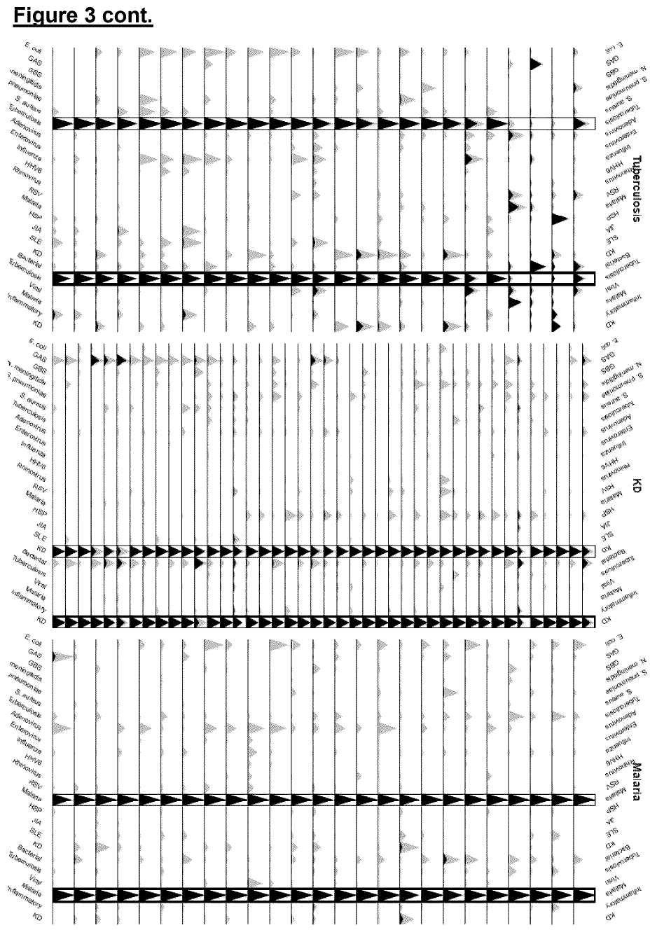

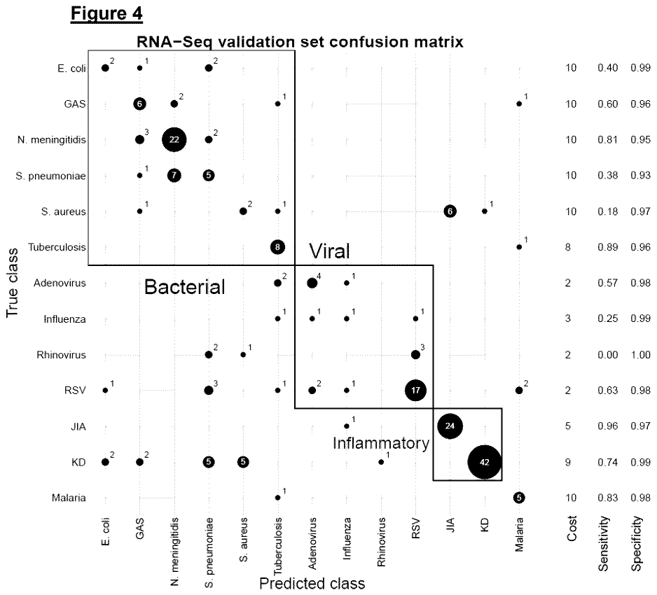

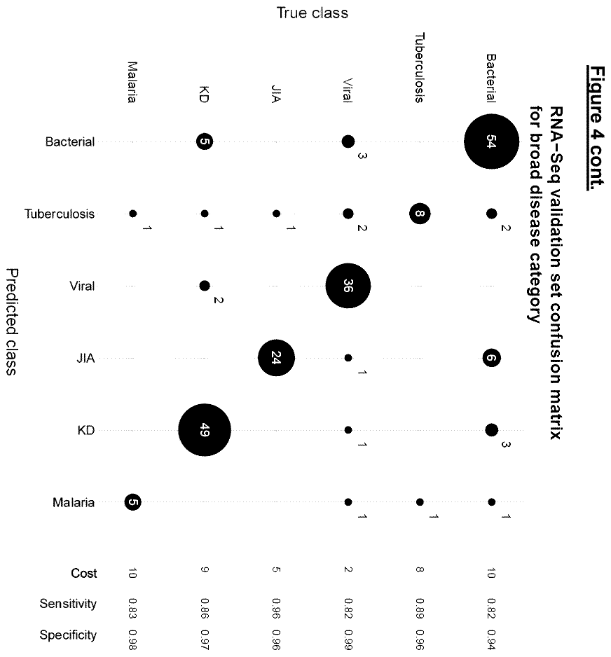

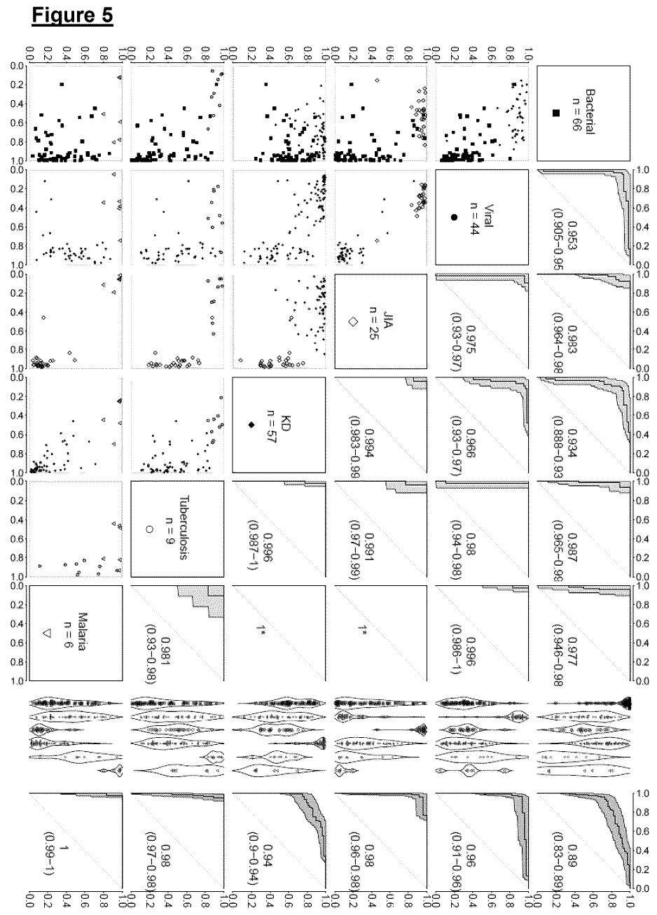

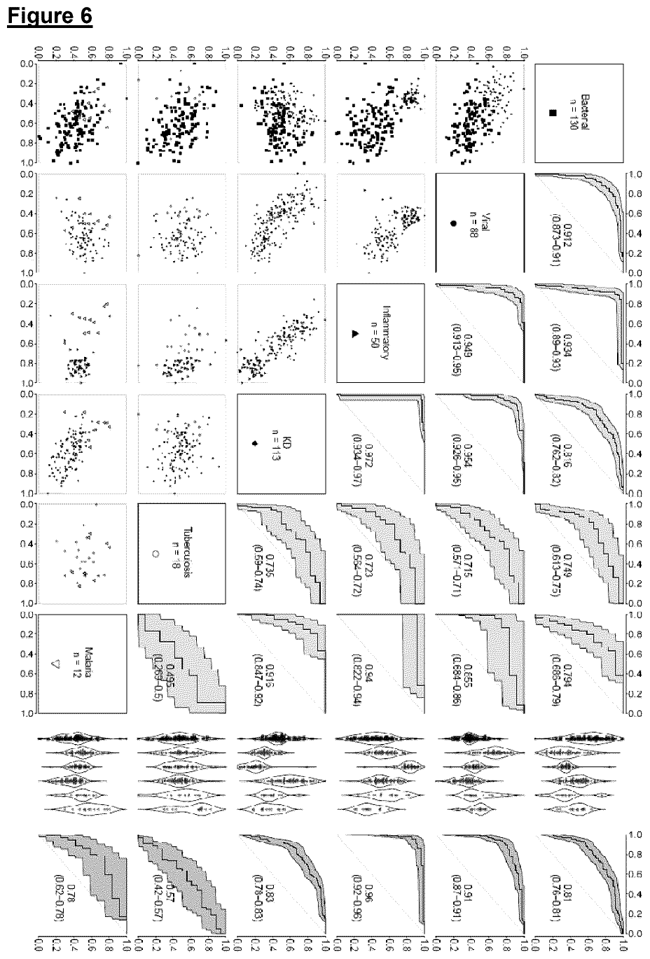

FIG. 3 shows predicted probabilities for all classes shown on a per patient basis in the microarray test set. Predicted probabilities are shown for all patients in the microarray test set. Patients are grouped by the clinically assigned (true) diagnosis in the 6 major disease groups: (A) Bacterial diagnosis (B) Viral diagnosis (C) Inflammatory diagnosis (D) Tuberculosis diagnosis (E) Kawasaki Disease diagnosis (D) Malaria diagnosis. For each disease group, each patient corresponds to a row in the relevant different panel. For each patient (row) predicted probabilities for each sample sum to 1 (black) or can sum to more than 1 (grey). The bold boxed column corresponds to the true disease category. The labels at the top and bottom of the plot correspond to the clinically-assigned disease diagnosis.

FIG. 4 shows the performance of the 145-transcript panel in the validation cohort. Multi-class confusion matrices for disease prediction in the RNA-Seq validation set for specific disease classes (A); and for broad disease classes (B). Circle area corresponds to number of patients. Specificities and sensitivities for the detection of each class were derived from discrete class predictions. Cost-weighting and point estimates for sensitivity and specificity for each prediction are shown on the right. E. coli: Escherichia coli, GAS: group A Streptococcus, N. meningititis: Neisseria meningitidis, S. pneumoniae: Streptococcus pneumoniae, S. aureus: Staphylococcus aureus, RSV: respiratory syncytial virus, JIA: juvenile idiopathic arthritis, KD: Kawasaki Disease.

FIG. 5 shows the RNA-Seq validation set predictions of broad disease classes. Pairwise and one-versus-all discrimination of broad disease categories. Scatter plots and ROC curves are shown for pairs of disease categories (columns 1-6). Each scatter plot shows the predicted probabilities for patients with one of a pair condition; conditions for each scatter plot are given on the diagonal, above (x axis) and to the right (y axis) of each plot. ROC curves show the performance when distinguishing each pair of conditions; conditions for each ROC plot are given on the diagonal, below and to the left of each plot. Separation of the pair of diseases is performed using the predicted probabilities of both classes where the decision threshold is defined by varying the gradient of a line p(class1)=m*p(class2). The rightmost panels (columns 7-8) show the predicted probabilities for each class (left) and the one-vs-all ROC curve defined using only these probabilities to distinguish the class in a one-versus-all comparison.

FIG. 6 shows predictions of the broad disease model in the RNA-Seq cohort. Model coefficients for the 161 probes were fitted over the combined training and test sets from the microarray dataset, predictions in the RNA-Seq cohort of each broad disease category are based on these coefficients as independent linear models without applying the softmax function. Scatter plot axes are adjusted such that zero and one correspond to lowest and highest values respectively. ROC curves for pairwise comparisons are derived from the ratio of predictions.

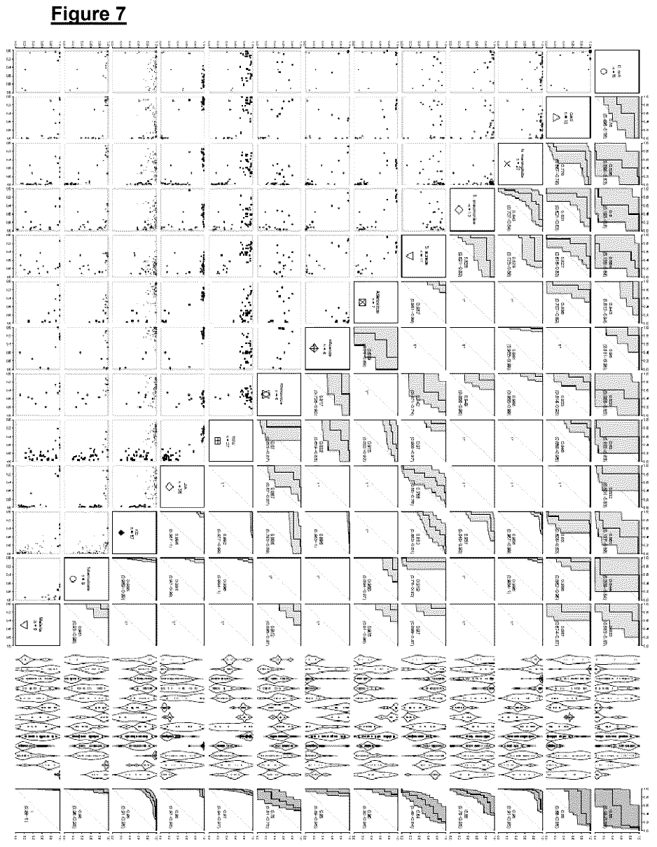

FIG. 7 shows RNA-Seq validation set predictions for specific disease categories. Pairwise and one-versus-all discrimination of classes. Scatter plots show the predicted probabilities for the pair of classes above (X axis) and to the right (Y axis) in samples over samples in each pair of groups. ROC curves show the performance when distinguishing each pair of classes (below and to the left) when using the predicted probabilities of both classes where the decision threshold is defined by varying the gradient of a line p(class1)=m*p(class2). The rightmost panels show the predicted probabilities for each class (left) and the ROC curve defined using only these probabilities to distinguish the class in a one-versus-all comparison. (* confidence intervals could not be calculated due to lack of overlap.

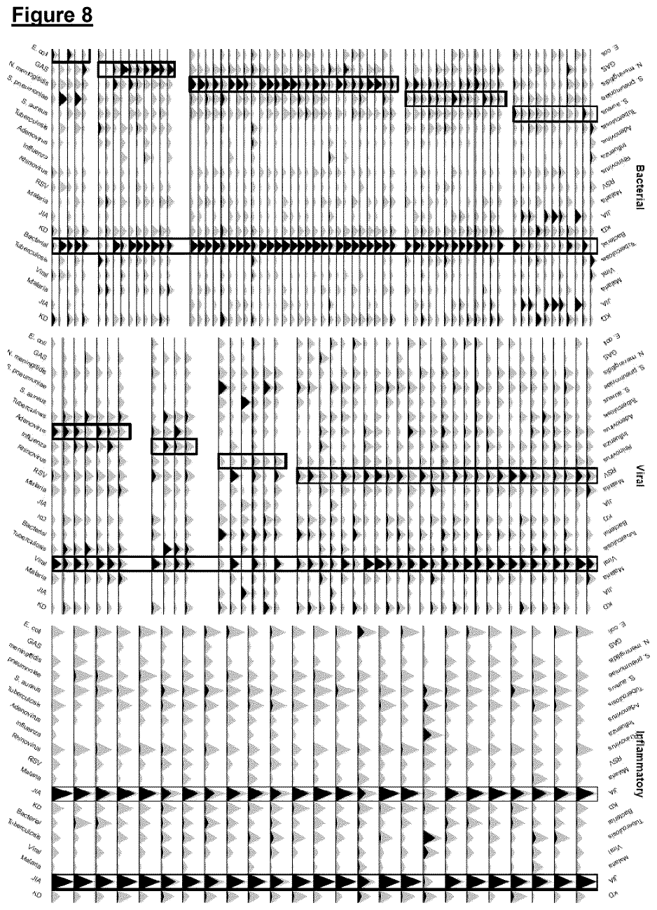

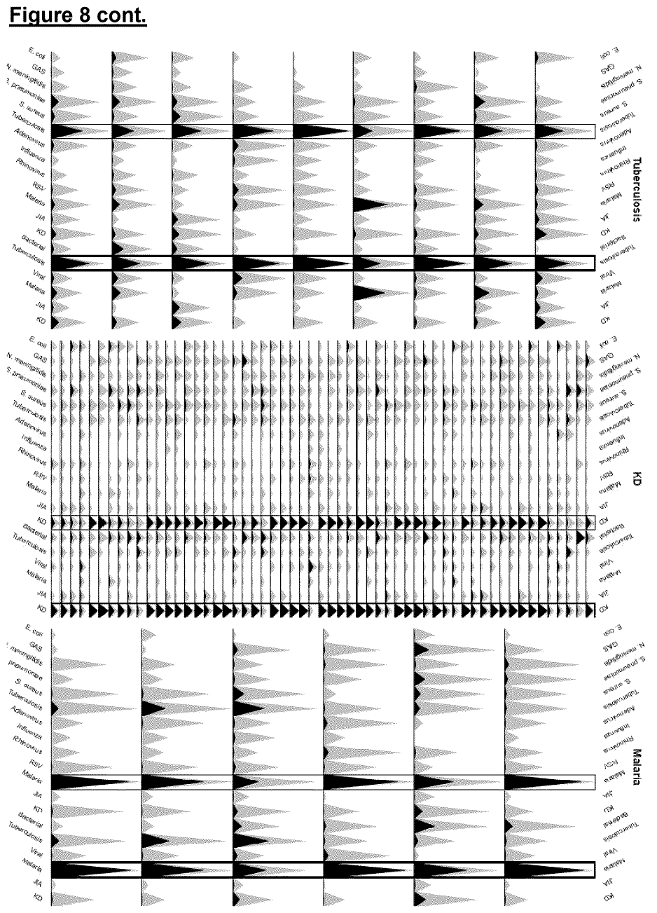

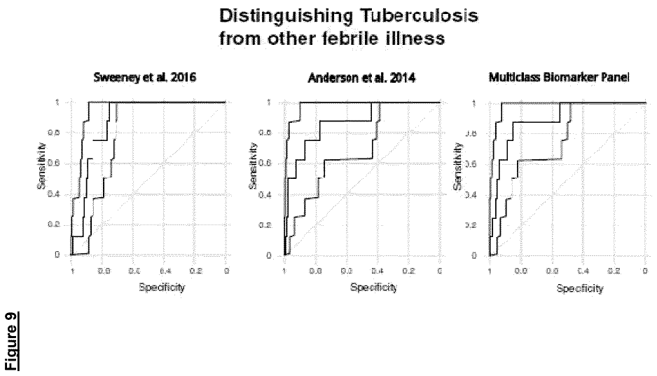

FIG. 8 shows predicted probabilities for all classes shown on a per patient basis in the RNA-Seq dataset. Predicted probabilities are shown for all patients in the RNA-Seq dataset. Patients are grouped by the clinically assigned (true) diagnosis in the 6 major disease groups: (A) Bacterial diagnosis (B) Viral diagnosis (C) Inflammatory diagnosis (D) Tuberculosis diagnosis (E) Kawasaki Disease diagnosis (D) Malaria diagnosis. For each disease group, each patient corresponds to a row in the relevant different panel. For each patient (row) predicted probabilities for each sample sum to 1 (black) or can sum to more than 1 (grey). The bold boxed columns correspond to the true disease category. The labels at the top and bottom of the plot correspond to the clinically-assigned disease diagnosis.

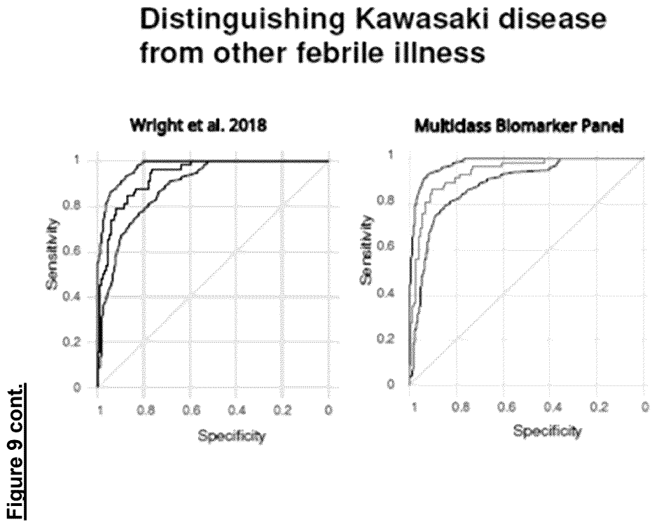

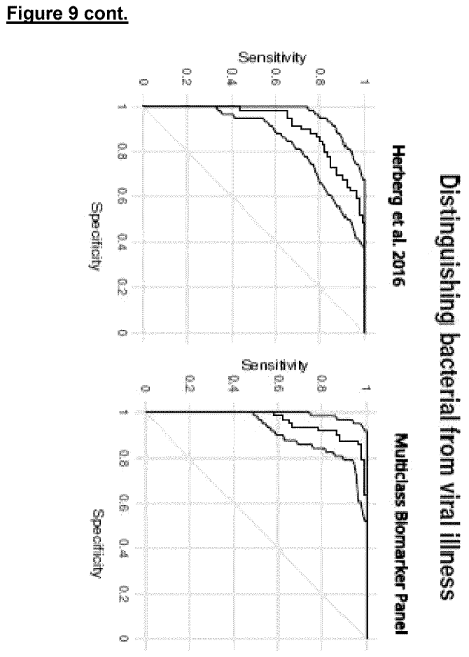

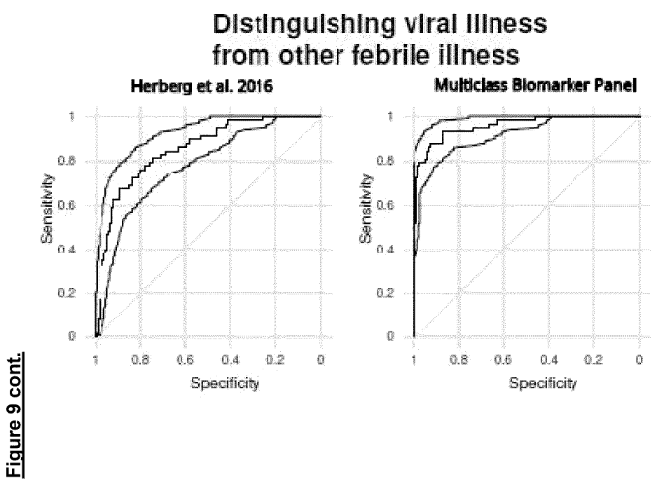

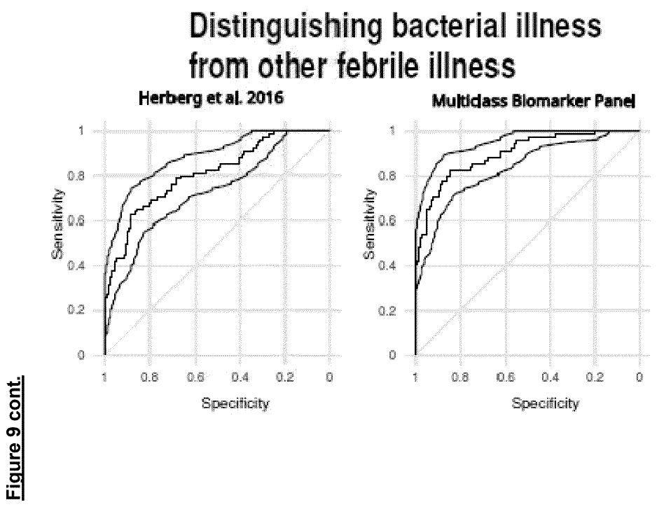

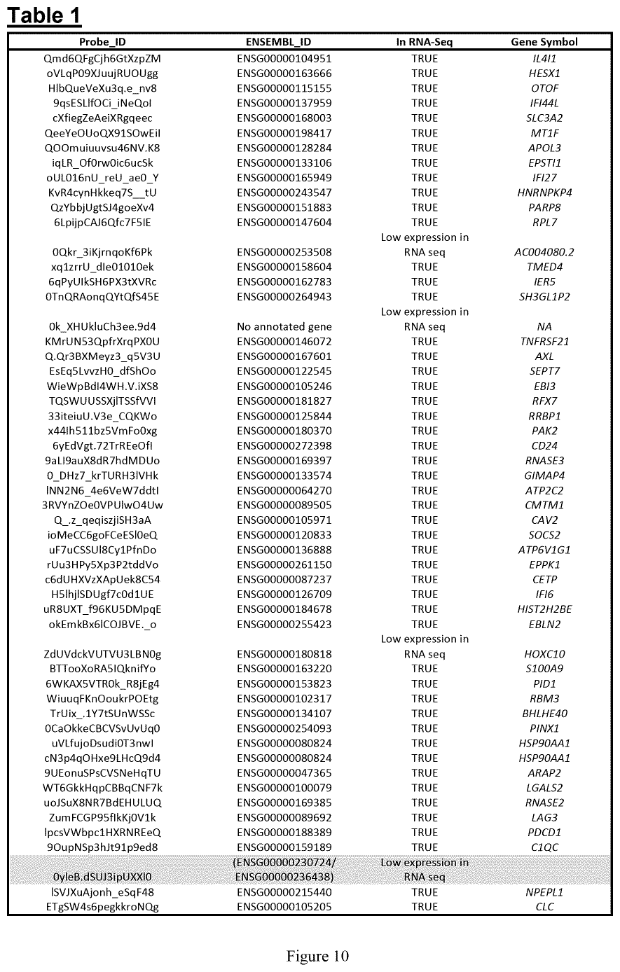

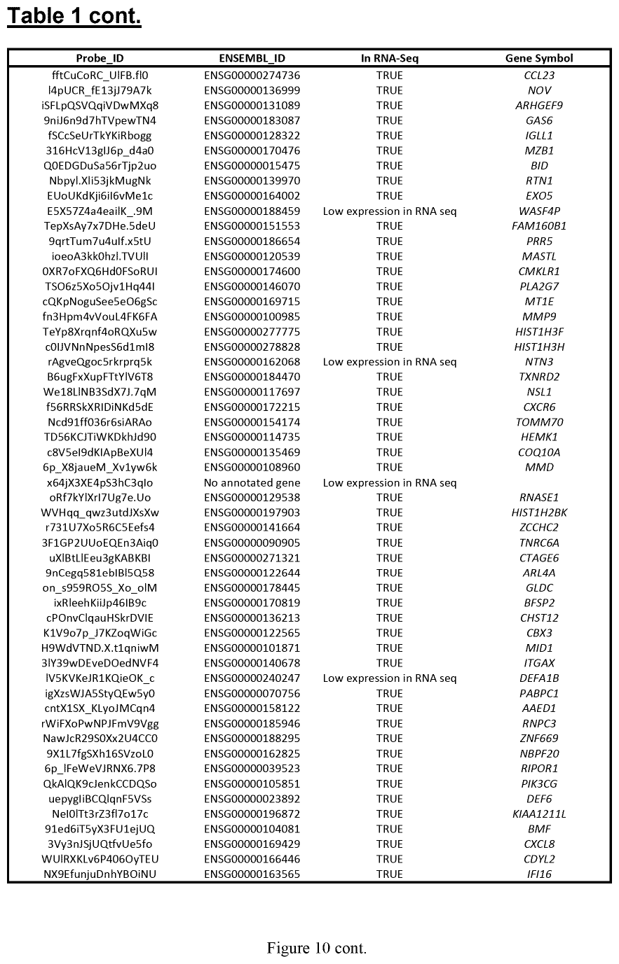

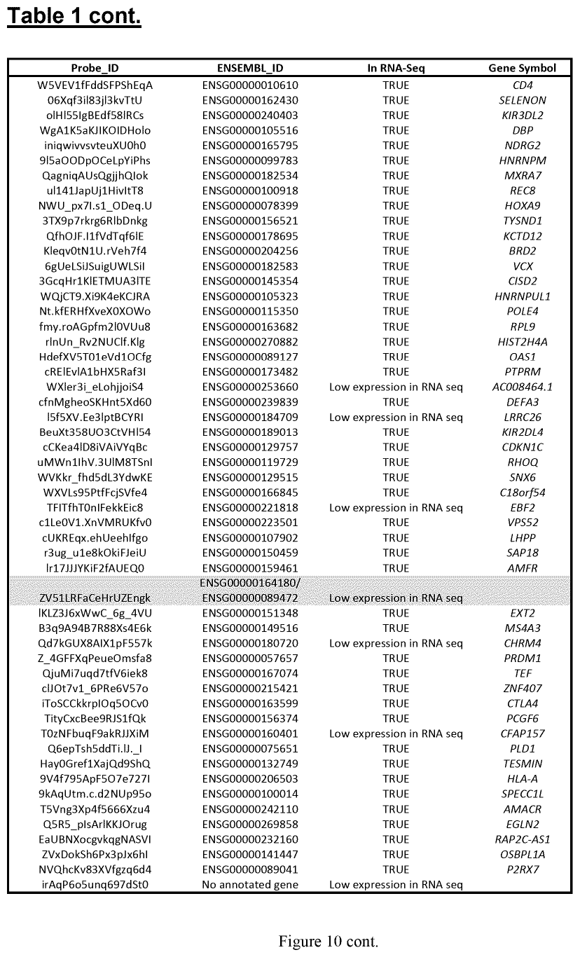

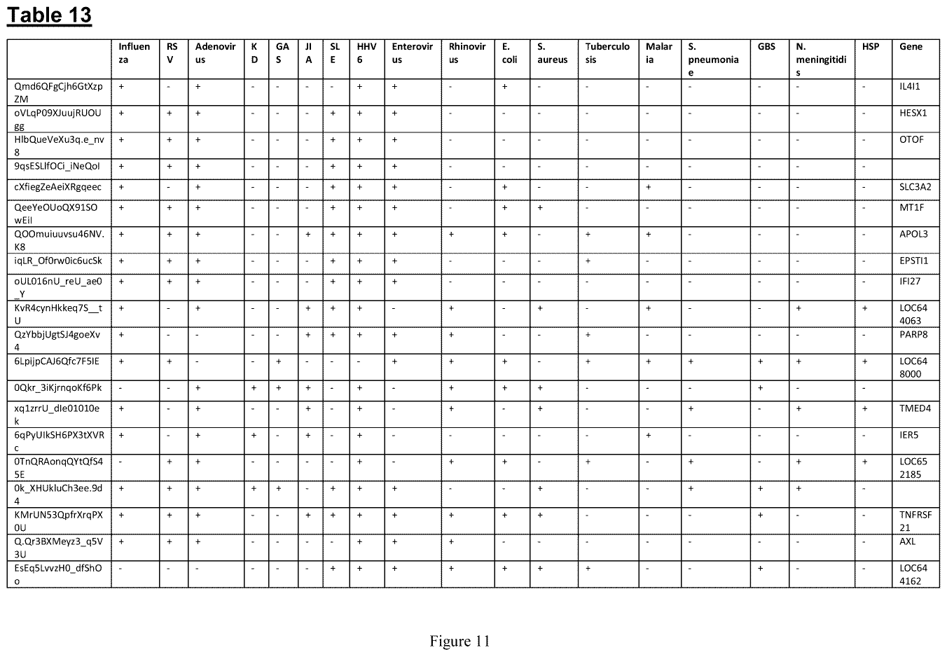

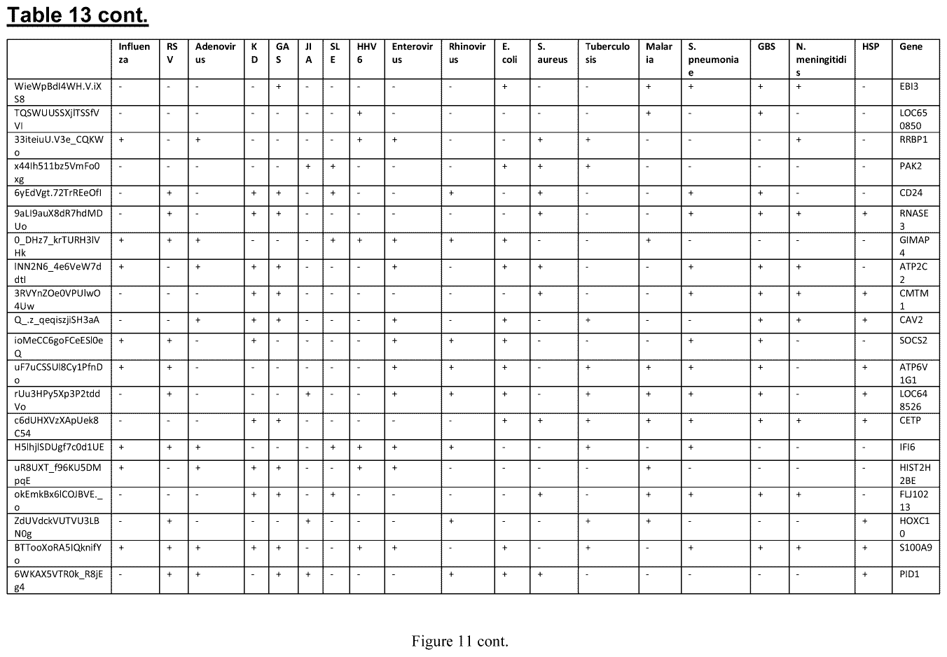

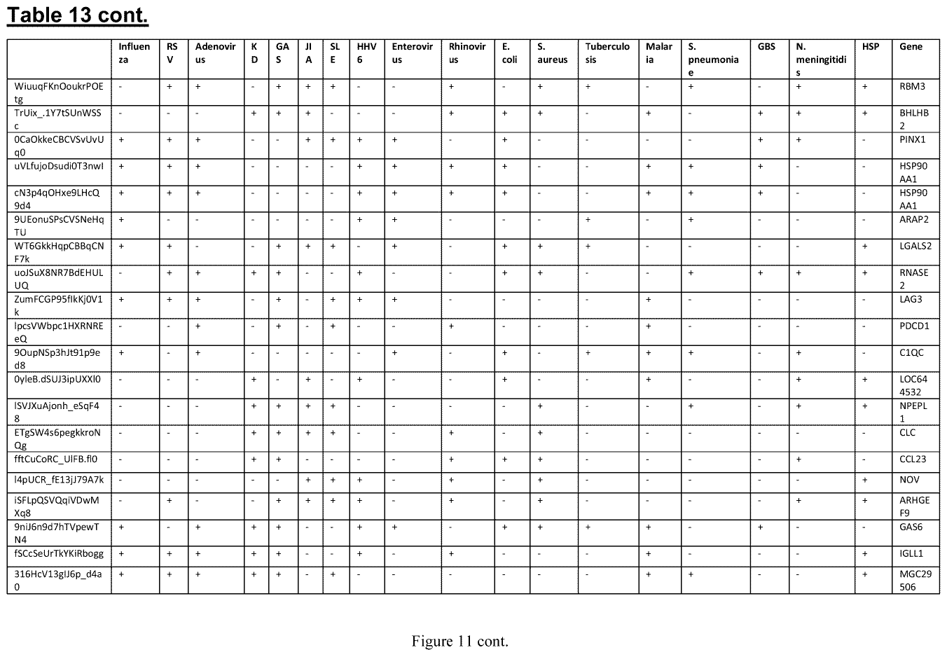

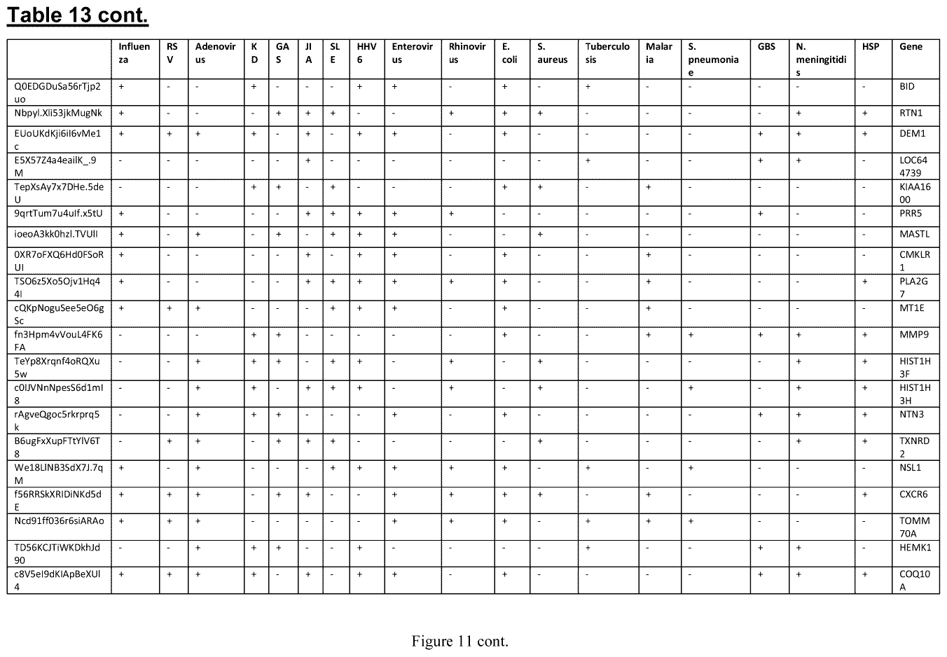

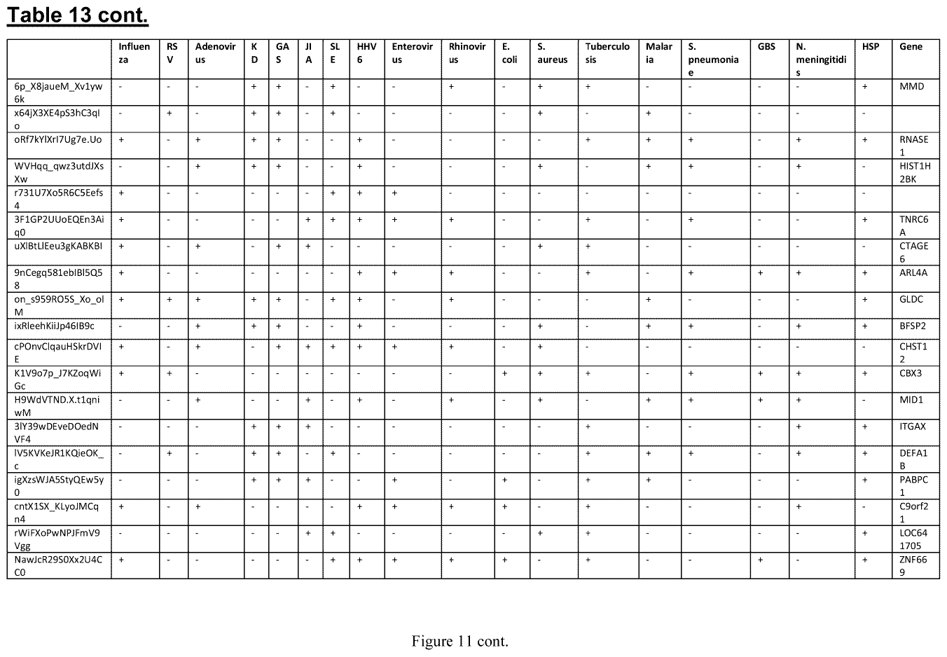

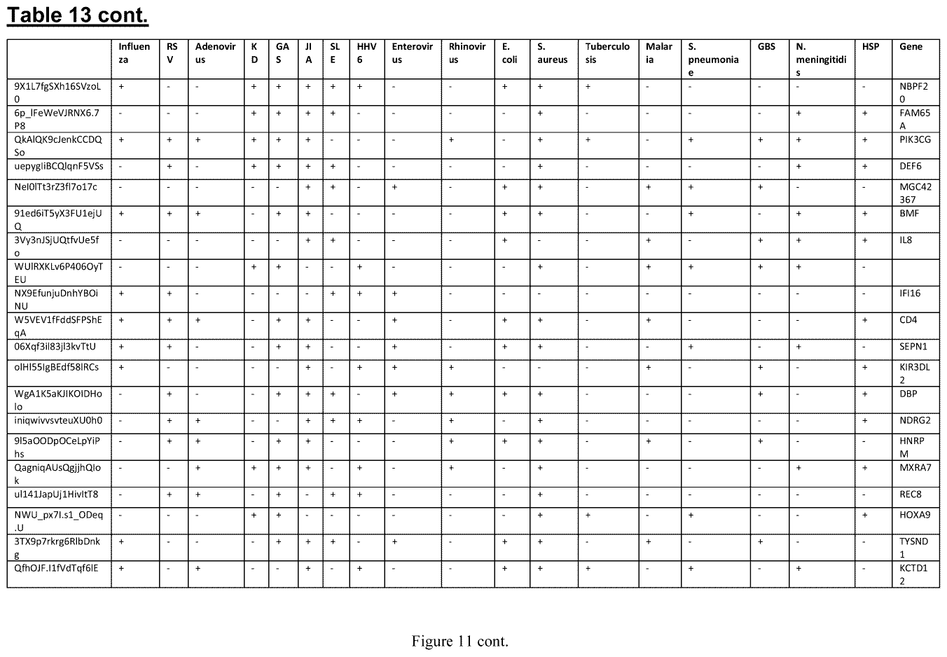

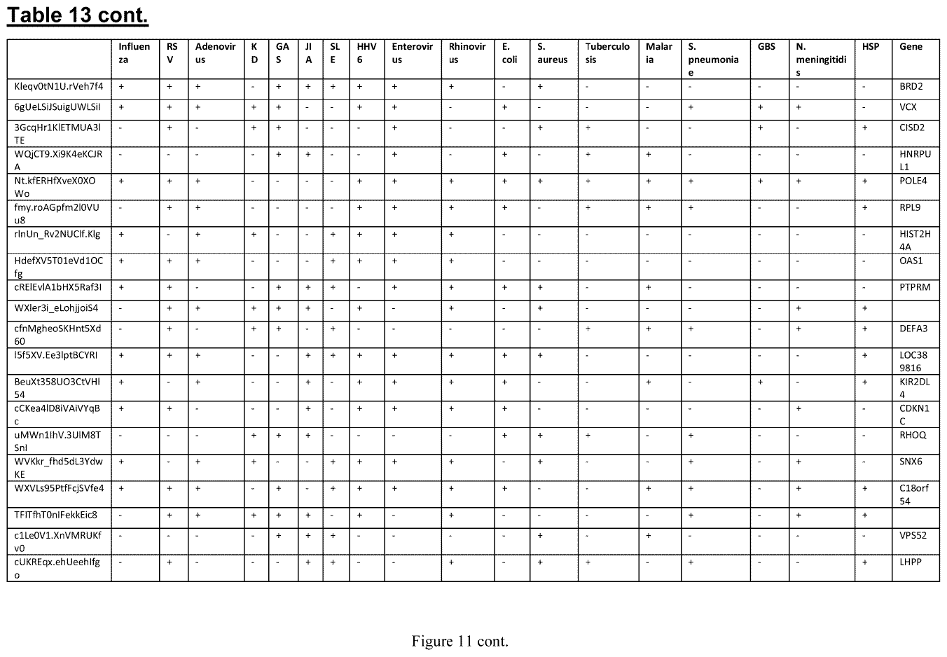

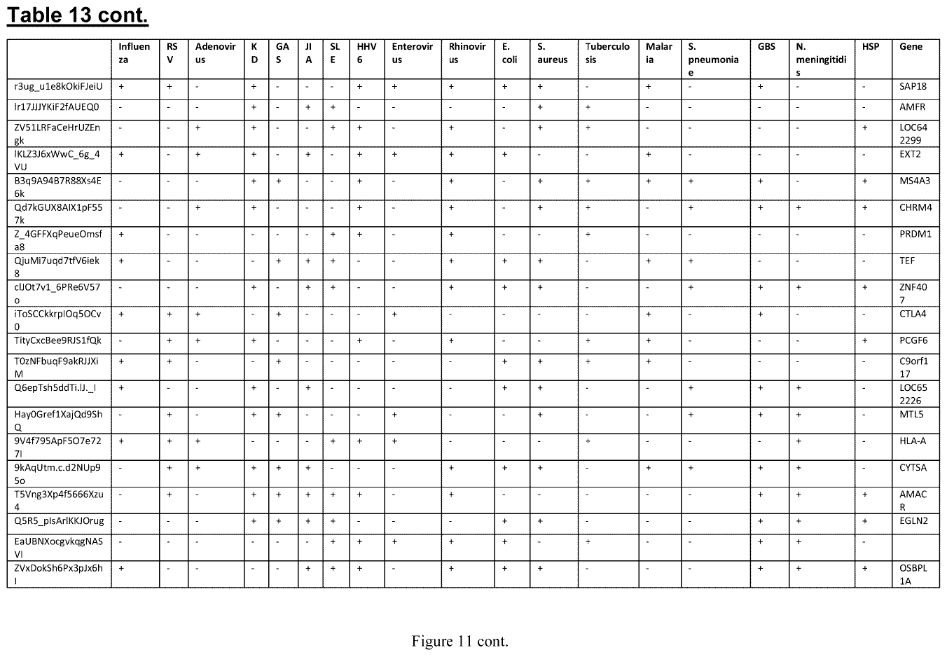

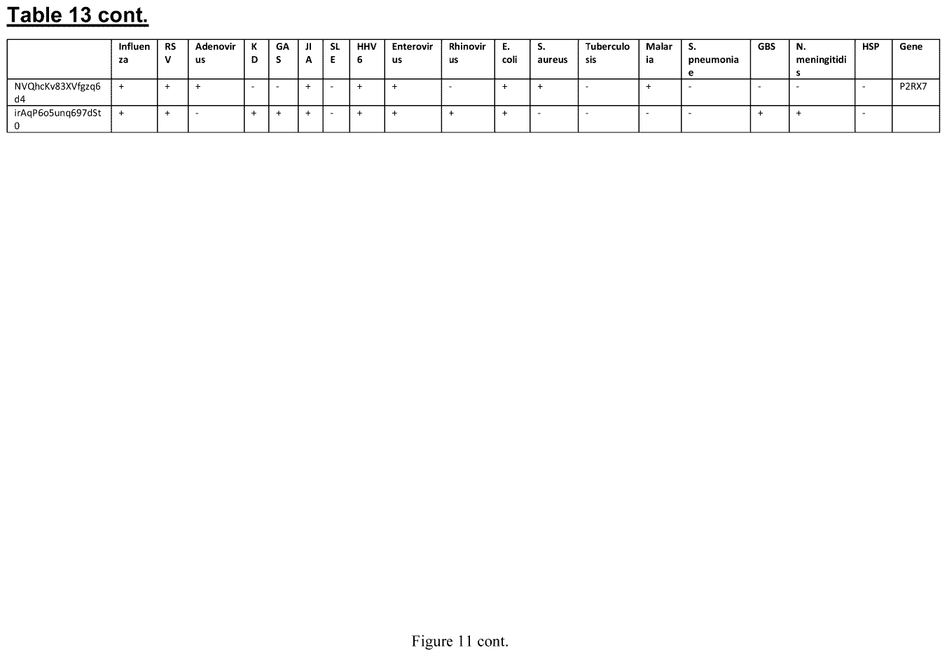

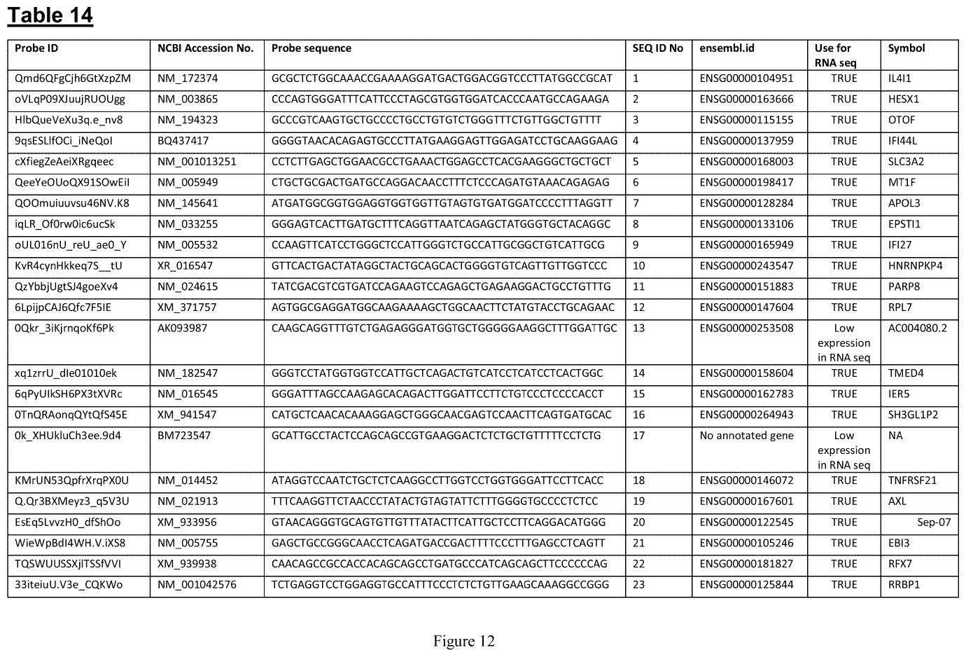

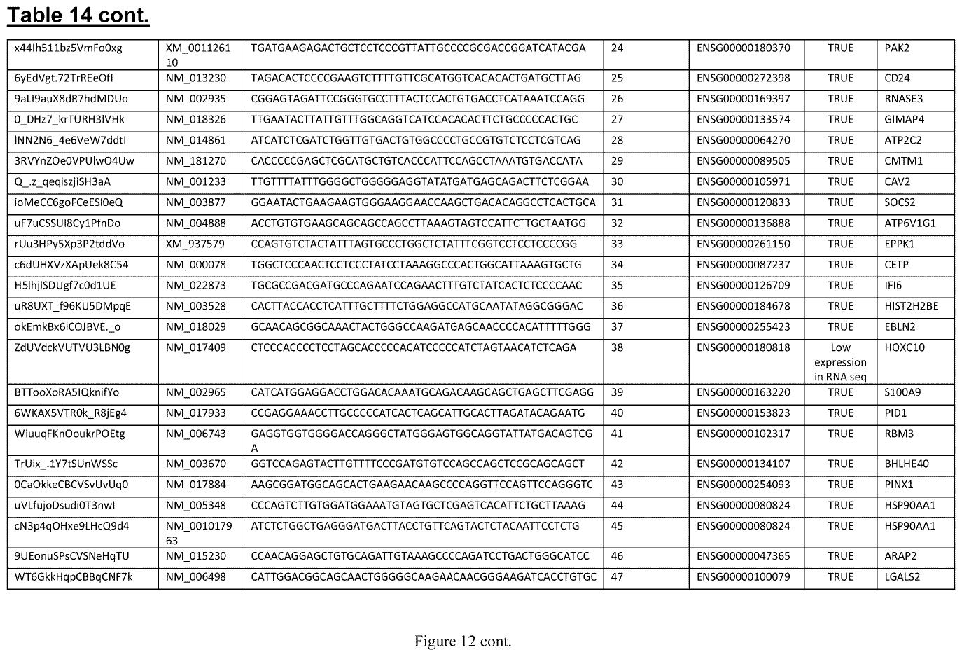

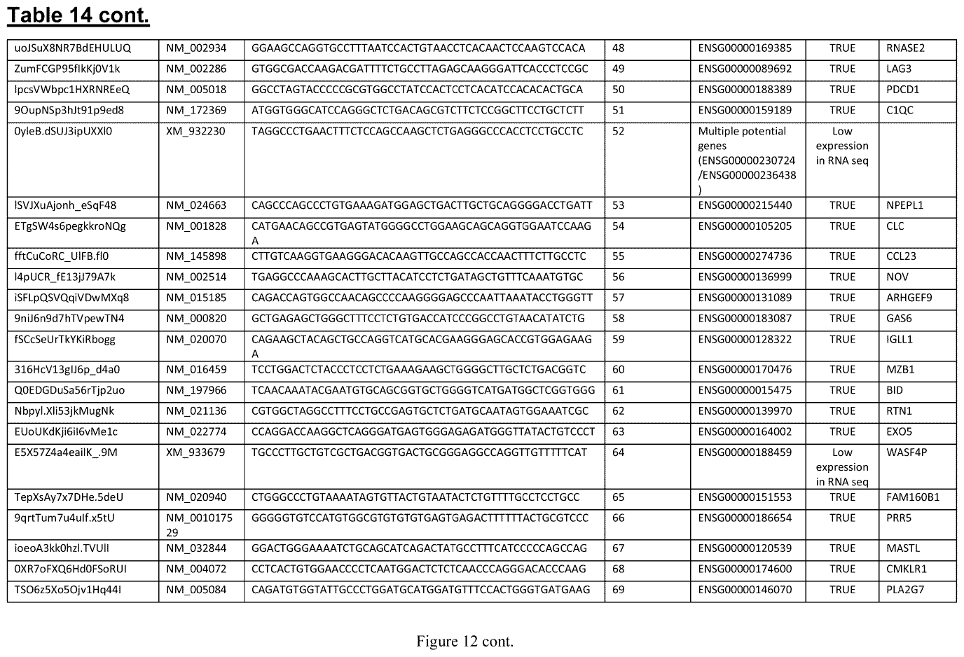

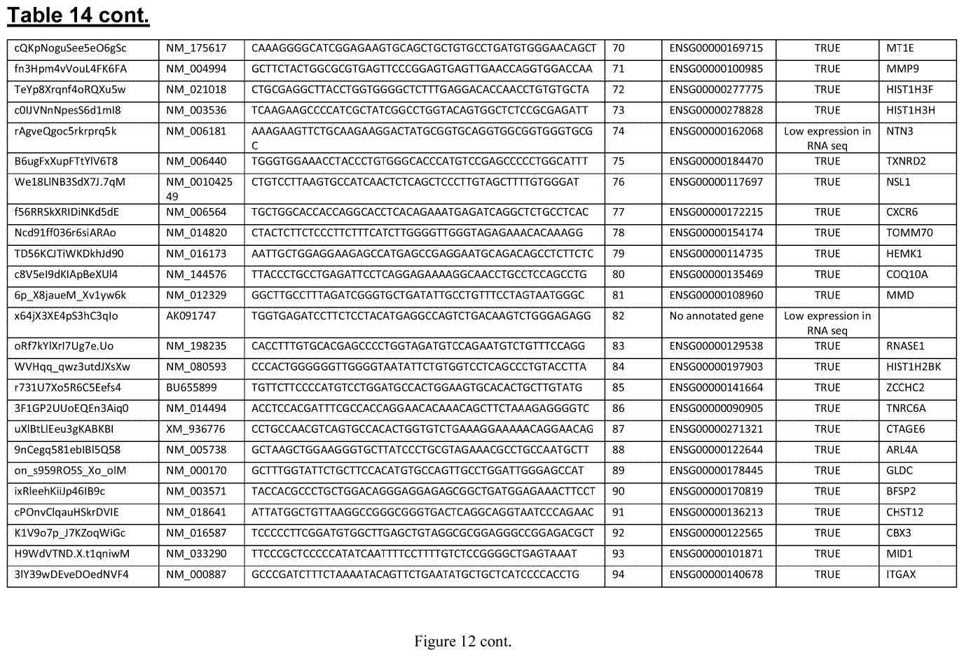

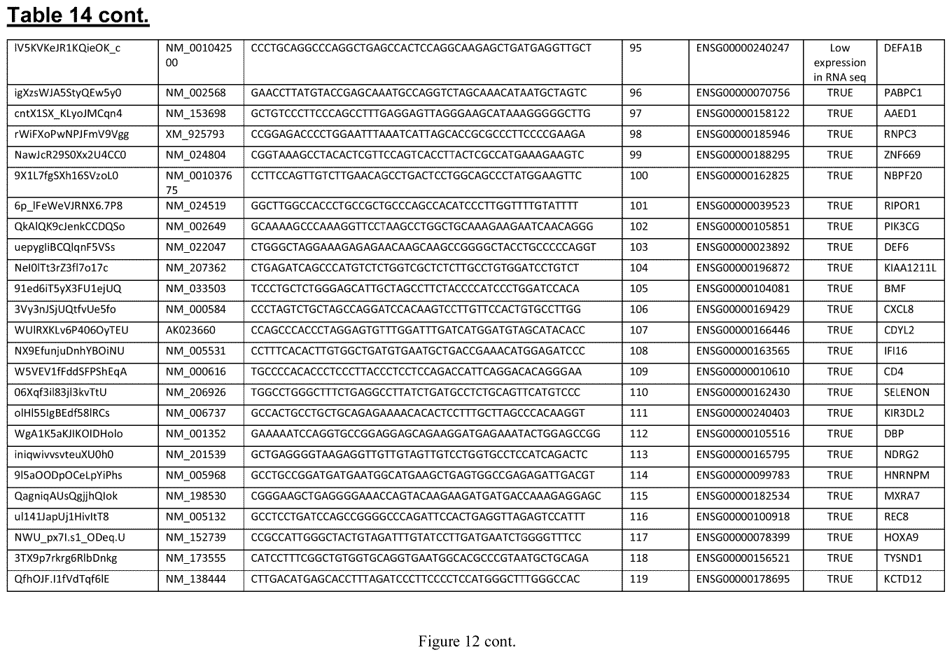

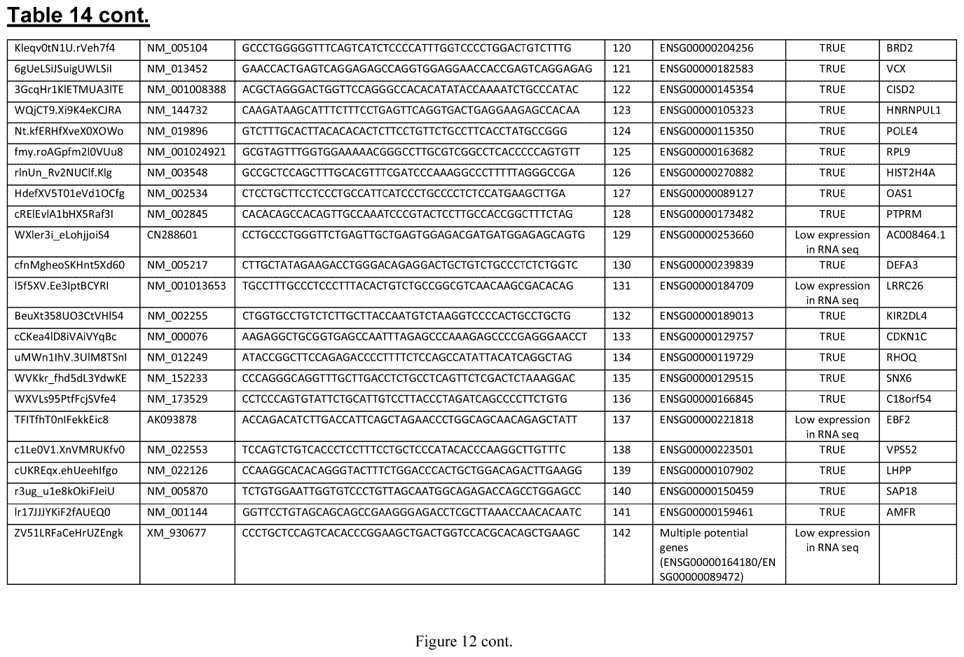

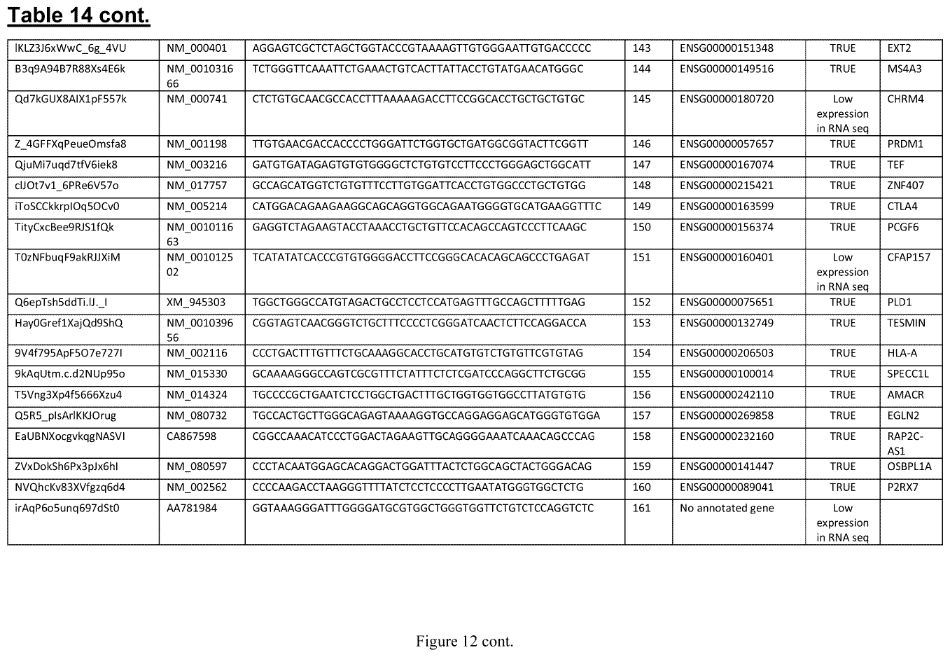

FIG. 9 shows the comparison of the multi-class RNA signature to previously published signatures of infectious disease. ROC curves and 95% confidence intervals of specificity are shown for the multi-class signature and previously reported signatures for: tuberculosis (A), Kawasaki disease (B) and for distinguishing bacterial and viral infection (C, D, E). The comparison to the bacterial-viral signature is split by the formulation of the classification problem. (C) shows a bacterial versus viral comparison where for the multi-class classifier the ratio of predicted probabilities for bacterial and viral infection are used. (D) and (E) show the problem as a viral versus all and bacterial versus all respectively with each using the corresponding component of the multi-class signature.

Table 1 shows the genes identified in the gene signature according to the invention. The table also shows the selected microarray probes and their overlap in the RNAseq data. The probes highlighted in grey correspond to multiple potential genes. Probe IDs are given as illumina nuIDs.

Table 13 lists the genes of the gene signature and indicates whether they are up-regulated (+) or down-regulated (−) depending on the disease of the subject. This modulation of gene expression is comparing one disease to the rest of the diseases.

Table 14 lists the probes used to identify the genes of the gene signature according to the invention. Table 14 identifies the NCBI Accession Number of the probes, as well as their nucleotide sequence (with the corresponding SEQ ID No).

### EXAMPLES

The inventors hypothesised that multiple infectious and inflammatory diseases could be simultaneously discriminated by a limited number of gene transcripts measured in patients' blood. To investigate this hypothesis, the inventors applied a multi-class feature selection and classification approach based on Lasso and Ridge regression to genome-wide RNA expression data from 1,212 febrile children in 12 publicly available gene expression microarray datasets, representing 18 disease categories, incorporating the clinical risks associated with incorrect diagnosis as a “cost weighting”. The inventors identified 161 transcripts that predicted the broad disease category (bacterial, viral, inflammatory, malaria, TB and KD) with high confidence, as well as specific causative pathogens and diseases. Cross-platform validation of the signature was performed in an independent cohort of patients (n=411) for whom gene expression was instead measured in whole blood by RNA-Seq. The data provide evidence that the pattern of expression of a single set of transcripts in each patient's blood can be surprisingly used to diagnose a wide range of infectious and inflammatory diseases.

### Materials and Methods

### Quantification and Statistical Analysis

Analyses were performed in R version 3.4.4.

### Microarray Data Pre-Processing

The inventors identified human Illumina gene expression micro-array datasets in National Institutes of Health Gene Expression Omnibus database and ArrayExpress, which included expression data from children with infectious and inflammatory diseases as well as healthy controls, as shown in Table 2.

TABLE 2
Samples included in the discovery gene expression microarray dataset.

| | | E. | | | | | | | N | S. | |
|---|---|---|---|---|---|---|---|---|---|---|---|
| Dataset_ID | Adenovirus | coli | Influenza | GAS | GBS | HSP | JIA | KD | meningitidis | pneumoniae | Rhinovirus |
| GSE73464 | 22 | 1 | 19 | 6 | 3 | 18 | 97 | 78 | 10 | 6 | 4 |
| GSE68004 | 19 | | | 17 | | | | 89 | | | |
| GSE65391 | | | | | | | | | | | |
| GSE64456 | | 55 | 48 | | 7 | | | | | | |
| GSE42026 | | | 12 | 3 | | | | | | 9 | |
| GSE40396 | 11 | 2 | | | | | | | | | 8 |
| GSE39941 | | | | | | | | | | | |
| GSE38900 | | | | | | | | | | | |
| GSE34404 | | | | | | | | | | | |
| GSE30119 | | | | | | | | | | | |
| GSE29366 | | | 19 | | | | | | | | |
| GSE22098 | | | | 12 | | | 1 | | | | |
| GSE63881 | | | | | | | | | | | |
| GSE25504 | | | | | | | | | | | |
| GSE38900 | | | | | | | | | | | |
| (V3/GPL6884) | | | | | | | | | | | |

| | | S. | Active | | | | | | |
|---|---|---|---|---|---|---|---|---|---|
| Dataset_ID | RSV | aureus | TB | SLE | Enterovirus | HHV6 | Malaria | Controls | Notes |
| GSE73464 | 27 | 1 | 5 | | | | | 62 | NA |
| GSE68004 | | | | | | | | 37 | NA |
| GSE65391 | | | | 135 | | | | 48 | Initial time |
| | | | | | | | | | points only |
| GSE64456 | | 3 | | | 51 | | | 19 | NA |
| GSE42026 | 6 | | | | | | | 33 | NA |
| GSE40396 | | 4 | | | 6 | 10 | | 22 | NA |
| GSE39941 | | | 95 | | | | 4 | 68 | NA |
| GSE38900 | 28 | | | | | | | 8 | Only V4/ |
| | | | | | | | | | GPL10558 |
| | | | | | | | | | array |
| GSE34404 | | | | | | | 93 | 62 | NA |
| GSE30119 | | 99 | | | | | | 43 | NA |
| GSE29366 | | | | | | | | 12 | NA |
| GSE22098 | | | | 69 | | | | 39 | NA |
| GSE63881 | | | | | | | | | No controls |
| GSE25504 | | | | | | | | | Premature |
| | | | | | | | | | infants |
| GSE38900 | | | | | | | | | excluded |
| (V3/GPL6884) | | | | | | | | | due to failed |
| | | | | | | | | | probes |

Only datasets where Illumina Beadchip arrays (V3, V4) were used to measure whole blood gene expression were included. Datasets were retrieved with getGEO, normalised using robust spline normalisation (RSN) from the lumi package and log transformed independently prior to batch correction. Probes common to all datasets were identified using lumiHumanIDMapping to map probes to Illumina nuIDs. Duplicate samples between datasets were identified using correlation structure and checked using patient characteristics. ComBat was performed using the R package SVA to correct for a batch internal to GSE72829 before using COCONUT, which assumes that healthy controls are drawn from the same distribution, to correct for batch effects between experiments.

Disease groups with fewer than 10 patients were excluded from the discovery set, as were cases in which a single causative pathogen was not identified or the diagnosis was uncertain. Stratified holdout was used to select the 25% of the data to be used in testing.

### Feature Pre-Filtering

Prior to feature selection, pre-filtering was performed to reduce the size of the search space and remove probes with little or no association with any of the diseases considered. To this end a differential expression analysis was performed with limma for all 153 pairwise disease comparisons. Probes with absolute log 2 fold change below 0.5 were discarded and the remaining probes for each comparison were ranked by p-value. Probes were selected from these lists in an iterative process until at least 2,000 probes were present. At each iteration the contribution of each probe was divided between the comparisons in which it was selected (i.e. a probe selected by 2 comparisons contributes a weight of 0.5 to each) in order that all comparisons were defined by similar numbers of discriminatory probes.

### Method Selection

In order to compare methods for performing the feature selection and classification, the inventors used stratified 10-fold cross-validation in the microarray training set, this was repeated 10 times by changing seed values. The inventors considered five different multivariate penalised regression methods, implemented here using glmnet: one-vs-all LASSO, one-vs-all LASSO followed by multinomial Ridge regression over the selected feature set, multinomial-LASSO, multinomial-LASSO+Ridge and multinomial relaxed LASSO. Nested cross validation was used to select hyper-parameters; when performing feature selection, the 1SE method was used and when refitting coefficients, the parameters were selected to minimise error. Performance was evaluated using mean weighted square error (MWSE) and mean size of the selected feature set. The inventors concluded that the one-versus-all approach was not feasible due to the identification of very large gene signatures with more highly correlated and redundant features. Of the multi-class approaches used, the LASSO+Ridge two-stage procedure obtained the smallest models with high predictive performance.

### LASSO+Ridge Hybrid

Penalised regression was performed on standardised expression values using the glmnet package in Bioconductor. Coefficients were grouped so that all coefficients for each feature were set to zero together. l1 and l2 penalised regression were combined into a two-stage procedure, referred to here as LASSO+Ridge, for which the LASSO (l1 penalty) was used to perform feature selection followed by a Ridge regression (l2 penalty) to refit the coefficients for the resulting feature set. This method has similarities with the Relaxed LASSO and LARS-OLS methods, which use LASSO and ordinary least squares (OLS) for the second stage respectively. For the LASSO+Ridge procedure the tuning parameters of LASSO (λ) and Ridge fits (φ) were selected using nested cross validation. At each λ of the LASSO regularisation path, genes with non-zero coefficients are used as input for a Ridge regression. For each Ridge regression the tuning parameter φ was selected to minimise the MWSE. λ was then selected to minimise model size such that the MWSE was within two standard errors of the minimum (2SE). Relative to LASSO and Relaxed LASSO, the LASSO+Ridge hybrid method had lower MWSE for each feature set, which resulted in smaller signatures with similar predictive accuracy.

### Cost and Rescalinq

Example weighting was used to bias the feature selection in order to prioritise the reduction of false negative error for diseases which are associated with greater immediate risk to the patient. These relative weights were defined for each disease class by a team of 5 paediatric infectious disease specialists to reflect: risk of negative outcome (e.g. death, organ damage), speed of disease progression and the availability of effective treatment (Table 3).

TABLE 3
Table of misclassification costs for the Discovery gene expression microarray dataset

| | | Cost/ | | |
|---|---|---|---|---|
| Disease | | disease | | |
| Group | Frequency | penalty | Justification | Weight |
| E. coli | 58 | 10 | Acute and life-threatening bacterial infection, | 0.17 |
| | | | requiring immediate diagnosis. | |
| GAS | 38 | 10 | Acute and life-threatening bacterial infection, | 0.26 |
| | | | requiring immediate diagnosis. | |
| GBS | 10 | 10 | Acute and life-threatening bacterial infection, | 1 |
| | | | requiring immediate diagnosis. | |
| N. meningitidis | 10 | 10 | Acute and life-threatening bacterial infection, | 1 |
| | | | requiring immediate diagnosis. | |
| S. pneumoniae | 15 | 10 | Acute and life-threatening bacterial infection, | 0.67 |
| | | | requiring immediate diagnosis. | |
| S. aureus | 107 | 10 | Acute and life-threatening bacterial infection, | 0.09 |
| | | | requiring immediate diagnosis. | |
| Tuberculosis | 100 | 8 | Slowly progressive infection, delay in diagnosis | 0.08 |
| | | | can result in more severe and disseminated | |
| | | | disease. | |
| Adenovirus | 52 | 2 | Usually self-limiting viral infection, no specific | 0.04 |
| | | | treatment, misclassification may result in | |
| | | | unnecessary antibiotic use. | |
| Enterovirus | 57 | 2 | Usually self-limiting viral infection, no specific | 0.04 |
| | | | treatment, misclassification may result in | |
| | | | unnecessary antibiotic use. | |
| Influenza | 98 | 3 | Sometimes severe, viral infection. Treatable by | 0.03 |
| | | | early initiation of antiviral therapy. | |
| HHV6 | 10 | 2 | Usually self-limiting viral infection, no specific | 0.2 |
| | | | treatment, misclassification may result in | |
| | | | unnecessary antibiotic use. | |
| Rhinovirus | 12 | 2 | Usually self-limiting viral infection, no specific | 0.17 |
| | | | treatment, misclassification may result in | |
| | | | unnecessary antibiotic use. | |
| RSV | 61 | 2 | Usually self-limiting viral infection, no specific | 0.03 |
| | | | treatment, misclassification may result in | |
| | | | unnecessary antibiotic use. | |
| HSP | 18 | 1 | Typically, self-limiting condition, with very few | 0.06 |
| | | | consequences of misdiagnosis at first | |
| | | | presentation. | |
| JIA | 98 | 5 | Chronic inflammatory condition, predominantly | 0.05 |
| | | | involving joints, delay to diagnosis incurs | |
| | | | significant healthcare costs and may result in | |
| | | | joint damage. | |
| SLE | 204 | 8 | Inflammatory condition, multiple organ | 0.04 |
| | | | involvement, delay to diagnosis incurs | |
| | | | significant healthcare costs and may result in | |
| | | | multiple end-organ organ damage. | |
| KD | 167 | 9 | Inflammatory condition, delay to diagnosis | 0.05 |
| | | | carries high risk of coronary artery aneurysms. | |
| Malaria | 97 | 10 | Acute and life-threatening parasitic infection, | 0.1 |
| | | | requiring immediate diagnosis. | |

Broad disease categories

| | | | | |
|---|---|---|---|---|
| Bacterial | 238 | 10 | Acute and life-threatening bacterial infection, | 0.04 |
| | | | requiring immediate diagnosis. | |
| Viral | 290 | 2 | Usually self-limiting viral infections, no specific | 0.01 |
| | | | treatment for most, misclassification may result | |
| | | | in unnecessary antibiotic use. | |
| Inflammatory | 320 | 5 | Mostly slowly progressive but earlier diagnosis | 0.02 |
| | | | may improve outcomes. | |
| KD | 167 | 9 | Inflammatory condition, delay to diagnosis | 0.05 |
| | | | carries high risk of coronary artery aneurysms. | |
| Malaria | 97 | 10 | Acute and life-threatening parasitic infection, | 0.1 |
| | | | requiring immediate diagnosis. | |
| Tuberculosis | 100 | 8 | Slowly progressive infection, delay in diagnosis | 0.08 |
| | | | can result in more severe and disseminated | |
| | | | disease. | |

The effect of adding class weights to a multinomial LASSO is to bias the feature set and weights towards reducing the false negative error for classes with worse potential outcomes, more rapid progression and available treatment; this also leads to an increase in the false positive error for these classes and the converse for diseases with smaller weights.

Weights were also modified to counteract the bias induced by differences in the numbers of samples in each group (class imbalance), as there was a 20:1 ratio between most and least abundant classes. This was done by updating class weights by dividing costs by the number of patients in each class.

### Performance

Classifier performance is shown using confusion matrices where discrete class predictions, for each patient, are the class with highest predicted probability. ROC curves were derived using the predicted probabilities for each class with the pROC package and trapezoidal calculation of AUC, the bootstrap methods were used for to derive confidence intervals and compare AUCs. Pairwise ROC curves were derived using the ratio of the predicted probabilities of the two classes.

### Subject Details

### Description of the Validation Study (RNA-Sea Dataset)

Patient recruitment: Patients were recruited as part of the European Union Childhood Life-threatening Infectious Disease Study (EUCLIDS https:/www.euclids-project.eu), a prospective, multicentre, cohort study conducted in six countries in Europe. Patients aged 1 month to 18 years with sepsis (or suspected sepsis) or severe focal infections, admitted to 98 participating hospitals in the UK, Austria, Germany, Lithuania, Spain, Switzerland and the Netherlands were prospectively recruited between Jul. 1, 2012, and Dec. 31, 2015. Febrile patients were recruited additionally with similar criteria in Spain (GENDRES network, Santiago de Compostela), in the Netherlands (Virgo cohort, JIA cohort), in the USA (Rady Children's Hospital-San Diego as described previously), and in Cape Town (Red Cross Children's hospital) between 2009 and 2013. Patients were recruited if they met the inclusion criteria of having febrile illness (temperature >=38° C.) of perceived sufficient severity to warrant blood testing or hospital admission and were <17 years of age. Patients were excluded if they had comorbidities or treatments likely to affect gene expression, including prior bone marrow transplant, immunodeficiency, or immunosuppressive treatment. Blood samples for RNA analysis were collected together with clinical blood tests at, or as close as possible to, presentation to hospital, irrespective of antibiotic use at the time of collection.

Diagnostic process: All patients underwent routine diagnostic investigations as part of clinical care in each hospital's microbiology and virology laboratories, including blood count and differential, C-reactive protein (CRP), blood chemistry, blood, and urine cultures, and cerebrospinal fluid (CSF) analysis where indicated. Throat swabs were cultured for bacteria, and viral diagnostics were undertaken on nasopharyngeal aspirates using multiplex PCR for common respiratory viruses. Chest radiographs and other tests were undertaken as clinically indicated. Patients were assigned to diagnostic groups using predefined criteria as described previously. The Definite Bacterial group included only patients with bacteria identified in a sample from a sterile site, and the Definite Viral group included only patients with culture, PCR or Immunofluorescent test—confirmed viral infection. Children were recorded as having juvenile idiopathic arthritis, Kawasaki disease, tuberculosis disease and malaria in the respective studies. Children in whom definitive diagnosis was not established were not used in this study.

Study conduct and oversight: Clinical data and patient samples were identified only by study number. Assignment of patients to clinical groups was made independent of those managing the patient clinically by consensus of two experienced clinicians, after review of the investigation results and using previously agreed definitions. Statistical analysis was conducted after the RNA expression data and clinical assignment databases had been locked. Written, informed consent was obtained from parents or guardians at all sites using locally approved permissions (St Mary's Research Ethics Committee (REC 09/H0712/58); Ethical Committee of Clinical Investigation of Galicia (CEIC ref 2010/015); Amsterdam, the Netherlands (NL41023.018.12 and NL34230.018.10); the University of California San Diego (Human Research Protection Program 140220); The Gambia Government/MRC Joint Ethics (Committee reference L2013.07V2); Cape Town, South Africa (HREC No 389/2017 linked to No 045/2008); Cantonal Ethis Committee Bern (KEK-029)).

Peripheral blood RNA sequencing: Whole blood was collected at the time of recruitment into PAXgene blood RNA tubes (PreAnalytiX, Germany), frozen, and later extracted. Library preparation and sequencing of 30 million 75 or 100 bp paired end reads was conducted using the Illumina's TruSeq RNA Sample Preparation Kit, ribosomal and globin RNA depletion was performed using the Illumina® Ribo-Zero Gold kit and HiSeq 4000 at The Wellcome Centre for Human Genetics.

### RNA-Sequencing Analysis

The RNA-Seq analysis pipeline consisted of: quality control using FastQC, MultiQC and annotations modified with BEDTools, alignment and read counting using STAR, SAMtools, FeatureCounts and version 89 ensembl GCh38 genome and annotation. Normalisation was performed using the DESeq2 method for estimating scale factors with a subsequent log transformation. The 161 microarray probes were mapped uniquely using BLAST to 155 genes, 10 of which were removed due to low read counts (unnormalised counts >5 in fewer than 10 samples) (Table 1). Refitting of the coefficients of the model was performed using Ridge regression on 50% of the dataset, the remainder was used for performance evaluation. The same split was used when retraining and testing comparator signatures (Table 5). Differential expression and enrichment analyses were performed using DESeq2 and g:Profiler.

### Results

Two datasets measuring whole blood gene expression from paediatric patients with febrile illness were used to investigate the potential of a multiclass approach to biomarker discovery. A dataset comprising 12 publicly available gene expression microarray datasets (n=1,212) was used for the discovery of a biomarker panel which was then applied to a newly generated RNA-Seq dataset (n=411).

### The Discovery Dataset

To explore the feasibility of using a limited number of RNA transcripts to classify febrile illness, the inventors merged and analysed publicly available microarray datasets. A comprehensive literature search, limited to Illumina Beadchip arrays, identified 12 datasets (GEO accession numbers: GSE73464, GSE68004, GSE65391, GSE64456, GSE42026, GSE40396, GSE39941, GSE38900, GSE34404, GSE30119, GSE29366, GSE22098), that measured gene expression in whole blood samples from both paediatric patients with acute febrile illnesses and appropriate controls (Table 2). The control samples in each dataset were used to perform batch correction with the COCONUT method.

Patients with multiple potentially causative pathogens and disease groups with fewer than 10 cases were excluded leaving 1,212 patients across 18 disease classes. Of these patients, 338 had bacterial infections caused by: Staphylococcus aureus (n=107), Streptococcus pneumoniae (n=15), group A Streptococcus (GAS) (n=38), group B Streptococcus (GBS) (n=10), Neisseria meningitidis (n=10), Escherichia coli (n=58) or Mycobacterium tuberculosis (n=100). 290 cases were due to viral infections, including: respiratory syncytial virus (RSV) (n=61), rhinovirus (n=12), human herpesvirus 6 (HHV6) (n=10), influenza virus (n=98), enterovirus (n=57) and adenovirus (n=52). 487 cases of inflammatory disease included systemic lupus erythematosus (SLE) (n=204), juvenile idiopathic arthritis (JIA) (n=98), Henoch-Schönlein purpura (HSP) (n=18) or Kawasaki disease (n=167). Malaria (n=97) was the only parasitic infection present in the datasets. The merged and batch corrected data were randomly split into subsets comprising 75% and 25% for training and testing respectively using stratified holdout to maintain class proportions.

### Identification of a Multi-Class Signature of Febrile Illness

In the discovery set, repeated cross-validation was performed in order to select the best method for deriving a multi-class signature of febrile illness (See Methods). Of the five multivariate penalised regression methods compared, LASSO+Ridge derived the smallest models with good classification performance while allowing cost-sensitivity. This is a two-stage method in which LASSO regression is used to perform feature selection followed by a Ridge regression to refit the coefficients and improve predictive performance.

An important consideration in the context of clinical diagnostics is the potential consequence of incorrect diagnosis. In order to incorporate the clinical consequences of misdiagnosis, the inventors applied a “cost-sensitive learning” approach by performing example weighting. Class weights were assigned by the consensus judgement of five independent paediatric infectious disease specialists to reflect the risks posed by each disease if untreated, the speed of disease progression and the availability of effective treatment. Weights were divided by the abundance of each class to offset the effect of class imbalance, as shown in Table 3.

The effect of incorporating these weights in the training process is to bias the feature set and coefficients to reduce the false negative error for high-risk groups at the expense of increasing the false negative error of low-risk groups.

The inventors applied multinomial LASSO+Ridge penalised regression in the 75% discovery set to identify an RNA transcript panel consisting of 161 probes for the discrimination of 18 disease classes. This set of probes was selected from the LASSO regularisation path at a value of lambda at which the cross validated mean square error (weighted by cost and class imbalance) for the Ridge regression was within 2 standard errors of the minimum.

### Test Set Predictions

As illustrated in FIG. 1, performance of the 161-transcript signature in the 250 microarray test set was tested over 18 specific disease classes (A) and over 6 broad disease classes (B). As shown, the gene signature resulted in a large number of correct predictions (true positives) for each disease category, with high specificity and sensitivity scores. The ability of the classifier to separate disease groups was also assessed for pairwise (one-versus-one) and one-versus-all discrimination on the basis of predicted probabilities (Table 4).

TABLE 4
Performance metrics for specific disease classes in the 25% microarray test set.
Sensitivity and specificity values correspond to the discrete class predictions
made by taking, for each patient, the class with highest predicted probability.

| | | | | Sensitivity for | Specificity for |
|---|---|---|---|---|---|
| | Number | | | discrete class | discrete class |
| Disease Group | of cases | AUCROC | CI (95%) | predictions | predictions |
| E. coli | 15 | 0.97 | 0.928-1 | 0.87 | 0.98 |
| GAS | 10 | 0.97 | 0.942-0.987 | 0.80 | 0.98 |
| GBS | 3 | 1 | 0.989-1 | 1.00 | 0.99 |
| N. meningitidis | 3 | 0.99 | 0.973-1 | 0.67 | 1.00 |
| S. pneumoniae | 4 | 0.91 | 0.826-0.985 | 0.25 | 1.00 |
| S. aureus | 27 | 0.94 | 0.879-1 | 0.78 | 0.99 |
| Tuberculosis | 25 | 0.97 | 0.932-1 | 0.88 | 1.00 |
| Adenovirus | 13 | 0.85 | 0.724-0.984 | 0.69 | 0.96 |
| Enterovirus | 15 | 0.93 | 0.891-0.976 | 0.67 | 1.00 |
| Influenza | 25 | 0.89 | 0.814-0.962 | 0.56 | 0.99 |
| HHV6 | 3 | 1 | 0.983-1 | 0.67 | 0.99 |
| Rhinovirus | 3 | 0.64 | 0.291-0.984 | 0.33 | 0.99 |
| RSV | 16 | 0.96 | 0.921-0.99 | 0.63 | 0.98 |
| HSP | 5 | 0.98 | 0.968-0.998 | 0.80 | 0.99 |
| JIA | 25 | 1 | 0.996-1 | 1.00 | 0.99 |
| SLE | 51 | 0.98 | 0.95-1 | 0.84 | 0.99 |
| KD | 42 | 0.99 | 0.984-1 | 0.95 | 0.97 |
| Malaria | 25 | 1 | NA | 1.00 | 1.00 |

Broad Clinical Categories with Immediate Clinical Implications

While the rapid identification of causative pathogens would be useful for optimal treatment and choice of antibiotics, clinical teams require a high degree of confidence in the broad disease category (i.e. viral, bacterial or inflammatory) to ensure potentially life-threatening conditions are not missed and to direct empiric treatment and appropriate subsequent investigations.

The inventors investigated whether the biomarker panel could also be used to make confident predictions of broad disease category. Refitting the coefficients for the 161 transcripts using multinomial Ridge regression allowed the panel to predict the broad disease categories: inflammatory disease, viral infection, bacterial infection, Kawasaki disease, malaria and tuberculosis (Table 5).

TABLE 5
AUCs, Sensitivities and Specificities for broad diagnostic categories in the microarray
test set. Sensitivity and specificity values correspond to the discrete class predictions
made by taking, for each patient, the class with highest predicted probability.

| | | | | Sensitivity | Specificity | |
|---|---|---|---|---|---|---|
| | | | | for discrete | for discrete | |
| | Number | AUC | CI | class | class | Specific |
| Disease | of cases | ROC | (95%) | predictions | predictions | diseases included |
| Tuberculosis | 25 | 0.97 | 0.938-1 | 0.88 | 1 | |
| KD | 42 | 0.99 | 0.986-1 | 0.95 | 0.97 | |
| Malaria | 25 | 1 | NA | 1 | 0.99 | |
| Bacterial | 62 | 0.93 | 0.902-0.966 | 0.87 | 0.92 | S. aureus (27), |
| | | | | | | E. coli (15), GAS |
| | | | | | | (10), S. pneumoniae |
| | | | | | | (4), GBS (3), |
| | | | | | | N. meningitidis (3) |
| Viral | 75 | 0.95 | 0.921-0.969 | 0.63 | 0.98 | Influenza (25), |
| | | | | | | RSV (16), |
| | | | | | | Enterovirus (15), |
| | | | | | | Adenovirus (13), |
| | | | | | | HHV6 (3), |
| | | | | | | Rhinovirus (3) |
| Inflammatory | 81 | 0.98 | 0.962-0.997 | 0.93 | 0.95 | SLE (51), JIA |
| | | | | | | (25), HSP (5) |

Although tuberculosis is a bacterial disease, it was considered as a separate class, as it requires very different clinical management from the other bacterial infections, and also induces distinct transcriptional responses. Similarly, Kawasaki disease, which also induces distinct transcriptional responses, was considered as a distinct class. Although epidemiological features suggest an infectious agent as the cause of Kawasaki disease, its aetiology remains unknown and treatment is directed at immunomodulation.

The resulting model accurately predicted the presence of these six disease classes both when considering the most likely class for each patient (FIG. 1B) and when considering classes independently (FIG. 2, Table 5) and these predictions allow the model to reflect the diagnostic classification used in clinical decision-making and simultaneously address multiple clinical questions.

The clinical teams can also be provided with the probabilities for each patient to belong in each class as an optimal input for decision-making (FIG. 3). As shown in FIG. 3, the predicted probabilities for the majority of the disease classes shown correspond to the patients' true disease category.

### Validation in an Independent Study Using RNA-Sequencing

The inventors evaluated the performance of the diagnostic signature in an independent patient cohort and using a different RNA quantification platform. The inventors used a newly generated dataset of whole blood RNA-Seq including 411 paediatric patients with a range of infectious or inflammatory diseases, covering all six broad diagnostic classes and 13 of the 18 specific diagnostic classes used in the discovery dataset (Demographic and clinical details Table 6 and Study details in Methods). Patients could be affected by more than one syndrome at the same time.

TABLE 6
RNA-Seq set demographics.

| | RNA-Seq dataset | | | | | |
|---|---|---|---|---|---|---|
| Characteristic | Bacterial | Viral | Inflammatory | TB | Malaria | KD |
| Number of patients | 130 | 88 | 50 | 18 | 12 | 113 |
| Age - mo. median (IQR) | 30 | 7 | 171 | 79 | 70 | 35 |
| | (9-65) | (2-20) | (132-200) | (43-93) | (51-93) | (18-56) |
| Male sex - no. (%) | 72 | 58 | 11 | 10 | 6 | 68 |
| | (55) | (66) | (22) | (56) | (50) | (60) |
| Population group | | | | | | |
| African | 25 | 5 | 0 | 1 | 12 | 7 |
| | (19.2) | (5.7) | | (5.6) | (100) | (6.2) |
| Asian | 5 | 2 | 1 | 0 | 0 | 16 |
| | (3.9) | (2.3) | (2.0) | | | (14.2) |
| European | 85 | 62 | 49 | 0 | 0 | 27 |
| | (65.4) | (70.5) | (98.0) | | | (23.9) |
| Latin American | 1 | 8 | 0 | 1 | 0 | 37 |
| | (0.8) | (9.1) | | (5.6) | | (32.7) |
| mixed/other/unknown | 14 | 11 | 0 | 16 | 0 | 26 |
| | (10.8) | (12.5) | | (88.9) | | (23.0) |
| Days from symptoms - | 2 | 5 | 264 (158-765)/ | 14 | 3 | 6 |
| median (IQR) | (1-4) | (2-7) | 877 (364-2095)* | (7-30) | (2-3) | (5-7) |
| Intensive care - | 69 | 17 | 0 | 0 | 0 | 2 |
| no. (%) | (53.1) | (19.3) | | | | (1.8) |
| Deaths - no. | 10 | 1 | 0 | 0 | 0 | 0 |
| CRP (mg/L) - | 203 | 6 | 10 | 60 | NA | 72 |
| median (IQR) | (111-281) | (3-18) | (3-44) | (51-69) | | (42-162) |
| Blood cell differential | | | | | | |
| Neutrophil %: | 75.0 | 29.0 | 51.3 | NA | 65.8 | 61.3 |
| median (IQR) | (59.9-85.7) | (16.8-48.7) | (42.8-59.4) | | (56.6-74.4) | (52.5-76.9) |
| Lymphocytes %: | 17.0 | 47.5 | 35.4 | 29.5 | 27.1 | 22.5 |
| median (IQR) | (9.3-27.9) | (28.5-60.3) | (29.8-45.0) | (25.0-35.5) | (18.8-34.4) | (11.8-30.2) |
| Monocyte %: | 5.88 | 7.7 | 8.4 | 5.0 | 6.8 | 5.0 |
| median (IQR) | (3.0-8.0) | (5.0-11.1) | (7.0-10.7) | (5.0-6.9) | (4.7-8.5) | (3.0-8.0) |
| Clinical Syndromes | | | | | | |
| Soft-tissue | 10 | 0 | 0 | 0 | 0 | 0 |
| Inflammatory | 0 | 0 | 50 | 0 | 0 | 113 |
| Gastrointestinal | 3 | 1 | 0 | 0 | 0 | 0 |
| Urinary tract | 8 | 0 | 0 | 0 | 0 | 0 |
| infection | | | | | | |
| Upper respiratory/ | 3 | 25 | 0 | 0 | 0 | 0 |
| Ear, Nose, Throat | | | | | | |
| Lower respiratory | 17 | 62 | 0 | 17 | 0 | 0 |
| tract | | | | | | |
| Central nervous | 38 | 0 | 0 | 0 | 0 | 1 |
| system involvement | | | | | | |
| Musculoskeletal | 7 | 0 | 0 | 0 | 0 | 0 |
| Other ^{‡} | 6 | 1 | 0 | 9 | 0 | 0 |
| Pathogen specific ^{†} | 3 | 0 | 0 | 0 | 12 | 0 |
| Sepsis | 76 | 0 | 0 | 0 | 0 | 0 |

IQR = Interquartile range, CRP: C-Reactive Protein, Ethnicity = self-reported ethnicity, TB: Tuberculosis; KD: Kawasaki disease;
*Relative to initial symptom onset for the first episode or exacerbations respectively;
^{†} Including: Scarlet fever, staphylococcal scalded skin and malaria;
^{‡} Including: central line-associated bloodstream infection, endocarditis, extra-pulmonary TB, facial palsy, pericarditis and status epilepticus.

The 161 microarray probes were mapped uniquely to 155 genes of which 10 did not have sufficient read counts in the RNA-Seq dataset for reliable quantification, leaving 145 genes in the panel in the RNA-Seq dataset (Table 1). Gene level read counts were normalised for sequencing depth with scaling factors calculated with DESeq2 followed by a log transformation. To account for the different quantification platform and smaller signature, the coefficients of the multi-class models for classifying both broad and specific disease class were refitted on a random selection of 50% the dataset using multinomial Ridge regression with class weighting, as shown in Table 7.

TABLE 7
Table of costs and weights for the RNA-Seq cohort

| | | Cost/Disease | |
|---|---|---|---|
| Disease Group | Frequency | penalty | Weight |
| E. coli | 10 | 10 | 1.00 |
| GAS | 19 | 10 | 0.53 |
| N. meningitidis | 54 | 10 | 0.19 |
| S. pneumoniae | 26 | 10 | 0.39 |
| S. aureus | 21 | 10 | 0.48 |
| Tuberculosis | 18 | 8 | 0.44 |
| Adenovirus | 14 | 2 | 0.14 |
| Influenza | 8 | 3 | 0.38 |
| Rhinovirus | 12 | 2 | 0.17 |
| RSV | 54 | 2 | 0.04 |
| JIA | 50 | 5 | 0.10 |
| KD | 113 | 9 | 0.08 |
| Malaria | 12 | 10 | 0.83 |

Broad disease categories

| | | | |
|---|---|---|---|
| Bacterial | 130 | 10 | 0.08 |
| Viral | 88 | 2 | 0.02 |
| Malaria | 12 | 10 | 0.83 |
| Tuberculosis | 18 | 8 | 0.44 |

The performance in the remaining 50% is shown for discrete class predictions (FIG. 4), using predicted probabilities for pairwise and one-versus-all comparisons (FIGS. 5, 6 and 7, and Tables 8 and 9) and for individual patients (FIG. 8). As illustrated in FIG. 4, the 145-transcript panel correctly classified the majority of the specific disease classes (FIG. 4A) and broad disease classes (FIG. 4B). Additionally, as illustrated in FIGS. 5-7, the gene signature performed well when distinguishing each pair of conditions, for both the broad and specific disease categories.

TABLE 8
Performance metrics for specific disease categories
in the test set of the RNA-Seq cohort.

| | | | | Sensitivity for | Specificity for |
|---|---|---|---|---|---|
| | Number | | | discrete class | discrete class |
| Disease | of cases | AUCROC | CI (95%) | predictions | predictions |
| E. coli | 5 | 0.55 | 0.158-0.94 | 0.40 | 0.99 |
| GAS | 10 | 0.85 | 0.66-1 | 0.60 | 0.96 |
| N. meningitidis | 27 | 0.95 | 0.91-0.994 | 0.82 | 0.95 |
| S. pneumoniae | 13 | 0.85 | 0.759-0.94 | 0.39 | 0.93 |
| S. aureus | 11 | 0.64 | 0.49-0.796 | 0.18 | 0.97 |
| Tuberculosis | 9 | 0.98 | 0.96-1 | 0.89 | 0.97 |
| Adenovirus | 7 | 0.97 | 0.923-1 | 0.57 | 0.99 |
| Influenza | 4 | 0.95 | 0.885-1 | 0.25 | 0.99 |
| Rhinovirus | 6 | 0.75 | 0.606-0.89 | 0.00 | 1.00 |
| RSV | 27 | 0.97 | 0.945-0.989 | 0.63 | 0.98 |
| JIA | 25 | 0.99 | 0.968-1 | 0.96 | 0.97 |
| KD | 57 | 0.95 | 0.913-0.981 | 0.74 | 0.99 |
| Malaria | 6 | 1 | 0.986-1 | 0.83 | 0.98 |

TABLE 9
RNA-Seq test set performance for broad disease categories

| | | | | Sensitivity | Specificity | |
|---|---|---|---|---|---|---|
| | | | | for discrete | for discrete | |
| | Number | | CI | class | class | Specific |
| Disease | of cases | AUCROC | (95%) | predictions | predictions | diseases included |
| Tuberculosis | 9 | 0.98 | 0.966-1 | 0.89 | 0.97 | |
| JIA | 25 | 0.98 | 0.961-1 | 0.96 | 0.96 | |
| KD | 57 | 0.94 | 0.9-0.975 | 0.86 | 0.97 | |
| Malaria | 6 | 1 | 0.986-1 | 0.83 | 0.99 | |
| Bacterial | 66 | 0.89 | 0.831-0.94 | 0.82 | 0.94 | E. coli (5), |
| | | | | | | GAS (10), N. |
| | | | | | | meningitidis |
| | | | | | | (27), S. |
| | | | | | | pneumoniae |
| | | | | | | (13), S. |
| | | | | | | aureus (11) |
| Viral | 44 | 0.96 | 0.913-1 | 0.82 | 0.99 | Adenovirus (7), |
| | | | | | | Influenza (4), |
| | | | | | | Rhinovirus (6) |

In addition, and although microarray and RNA-Seq rely on very different quantification approaches, the inventors assessed the performance of the broad disease classifier in the RNA-Seq dataset without retraining the coefficients in the RNA-Seq dataset. The coefficients were refitted using ridge regression in the complete microarray dataset. This model was then used to make predictions on the RNA-Seq dataset after applying limma voom transformation to the DESeq2 depth normalised RNA-Seq count data (FIG. 6).

The utility of a diagnostic test is highly dependent on the prevalence of disease in the population on which it is being used, however since a multi-class diagnostic panel could be applied in different clinical contexts, values for specificity, positive predictive value and negative predicted value are shown for four illustrative scenarios of disease prevalence in different populations in Table 10.

TABLE 10
Illustrative scenarios for evaluation of diagnostic
performance of the high-level multiclass signature.

| Scenario | | Bacterial | Viral | Inflammatory | TB | Malaria | KD |
|---|---|---|---|---|---|---|---|
| All | Prevalence (%) | 16.6 | 16.6 | 16.6 | 16.6 | 16.6 | 16.6 |
| diseases | PPV | 0.840 | 0.959 | 0.894 | 0.748 | 0.848 | 0.926 |
| at equal | NPV | 0.964 | 0.965 | 0.992 | 0.977 | 0.967 | 0.972 |
| prevalence | Sensitivity | 0.818 | 0.818 | 0.960 | 0.889 | 0.833 | 0.860 |
| | Specificity | 0.969 | 0.993 | 0.977 | 0.940 | 0.970 | 0.986 |
| Febrile | Prevalence (%) | 20 | 70 | 3 | 1 | 1 | 5 |
| children | PPV | 0.759 | 0.997 | 0.458 | 0.176 | 0.294 | 0.632 |
| undergoing | NPV | 0.954 | 0.701 | 0.999 | 0.999 | 0.998 | 0.993 |
| blood | Sensitivity | 0.818 | 0.818 | 0.960 | 0.889 | 0.833 | 0.860 |
| tests in | Specificity | 0.935 | 0.994 | 0.965 | 0.958 | 0.980 | 0.974 |
| European | | | | | | | |
| emergency | | | | | | | |
| departments | | | | | | | |
| Hospitalized | Prevalence (%) | 36 | 15 | 3 | 1 | 1 | 44 |
| children | PPV | 0.858 | 0.888 | 0.444 | 0.239 | 0.455 | 0.950 |
| with fever | NPV | 0.900 | 0.968 | 0.999 | 0.999 | 0.998 | 0.897 |
| without | Sensitivity | 0.818 | 0.818 | 0.960 | 0.889 | 0.833 | 0.860 |
| source | Specificity | 0.924 | 0.982 | 0.963 | 0.971 | 0.990 | 0.965 |
| (<7 days | | | | | | | |
| duration) | | | | | | | |
| Hospitalized | Prevalence (%) | 40 | 16 | 28 | 5 | 1 | 10 |
| children | PPV | 0.943 | 0.974 | 0.871 | 0.567 | 0.353 | 0.798 |
| with fever | NPV | 0.889 | 0.966 | 0.984 | 0.994 | 0.998 | 0.984 |
| of unknown | Sensitivity | 0.818 | 0.818 | 0.960 | 0.889 | 0.833 | 0.860 |
| origin >7 | Specificity | 0.967 | 0.996 | 0.944 | 0.964 | 0.985 | 0.976 |
| days | | | | | | | |
| duration | | | | | | | |
| African | Prevalence (%) | 15 | 70 | 1 | 3 | 10 | 1 |
| Outpatient | PPV | 0.716 | 0.999 | 0.245 | 0.332 | 0.795 | 0.274 |
| children | NPV | 0.967 | 0.702 | 1.000 | 0.996 | 0.981 | 0.999 |
| with fever | Sensitivity | 0.818 | 0.818 | 0.960 | 0.889 | 0.833 | 0.860 |
| | Specificity | 0.943 | 0.999 | 0.970 | 0.945 | 0.976 | 0.977 |

In each scenario of Table 10, the prevalence of the six high level diagnostic groups has been extrapolated from published data with the simplifying assumption that these groups are mutually exclusive and together account for 100% of diagnoses. Where prevalence of a disease group could not be calculated from the original data, we made estimates based on our own clinical experience and set minimum prevalence for any group to 1%. Performance metrics taking account of disease prevalence scenarios were calculated from the microarray test set confusion matrix of broad disease categories. Rows of the confusion matrix were divided by the number of test set samples and multiplied by each of the prevalence scenarios prior to calculating the metrics.

The inventors performed differential expression analyses between each broad disease category and the other disease groups using DESeq2 and the enrichment analysis for the different comparisons using g:Profiler, of gene ontology (biological pathways) and Reactome terms.

Benchmarking with Previously Published One Vs all Signatures

There are no previously reported transcriptional panels which can simultaneously distinguish multiple causes of fever in children against which to benchmark performance. The inventors therefore compared the performance of their multi-class biomarker panel to four previously reported binary classification signatures for the classification of paediatric febrile illness: tuberculosis, Kawasaki disease and for distinguishing bacterial from viral infections (Sweeney et al. 2016, Herberg et al. 2016, Wright et al. 2018, Anderson et al. 2014). Since some of the microarray datasets were used for binary signature derivation, performance was only compared in the RNA-Seq dataset for comparison fairness. The coefficients of each linear model were refitted using the same 50% of the RNA-Seq dataset and performance was evaluated using Receiver Operating Characteristic (ROC) curves on the remaining 50%.

As shown in FIG. 9 and Tables 11 and 12, the improvement relative to the bacterial-viral signature was significant for the identification of viral infection (viral-versus-all (p=0.007))—and bacterial infection (bacterial-versus-all (p=0.007)), Additionally, the multi-class biomarker panel performed better in terms of AUC than the single class Sweeney signature for tuberculosis (p=0.03).

TABLE 11
AUCROC values for previously published signatures
of paediatric infectious disease and the multiclass
signature in the RNA-Seq test set.

| | | Test | | |
|---|---|---|---|---|
| | | set | | |
| Signature | comparison | cases | AUCROC | CI (95%) |
| MCS- Tuberculosis | Tuberculosis- | 9 | 0.98 | 0.97-1 |
| | vs-ALL | | | |
| Sweeney et al. | Tuberculosis- | 9 | 0.93 | 0.90-0.97 |
| 2016 | vs-ALL | | | |
| Anderson et al. | Tuberculosis- | 9 | 0.93 | 0.88-0.98 |
| 2014 | vs-ALL | | | |
| MCS-Kawasaki | Kawasaki-vs- | 57 | 0.94 | 0.90-0.97 |
| | ALL | | | |
| Wright et al. 2018 | Kawasaki-vs- | 57 | 0.91 | 0.87-0.95 |
| | ALL | | | |
| MCS-Bacterial | Bacterial-vs- | 66 | 0.95 | 0.90-1 |
| | Viral | | | |
| Herberg et al. | Bacterial-vs- | 66 | 0.93 | 0.88-0.98 |
| 2016 | Viral | | | |
| MCS-Viral | Viral-vs-ALL | 44 | 0.96 | 0.91-1 |
| Herberg et al. | Viral-vs-ALL | 44 | 0.86 | 0.81-0.91 |
| 2016 | | | | |
| MCS-Bacterial | Bacterial-vs- | 66 | 0.89 | 0.83-0.94 |
| | ALL | | | |
| Herberg et al. | Bacterial-vs- | 66 | 0.80 | 0.74-0.87 |
| 2016 | ALL | | | |

TABLE 12
Performance comparison between multi-class signature and previously published signatures
of paediatric infectious disease over respective comparisons in the RNA-Seq test set.

| | | AUC - | AUC - | | |
|---|---|---|---|---|---|
| Pairwise comparison | comparison | MSC | comparator | Z | p |
| MCS-tuberculosis/Sweeney et al. 2016 | Tuberculosis-vs-All | 0.98 | 0.93 | 2.43 | 0.015 |
| MCS-tuberculosis/Anderson et al. 2014 | Tuberculosis-vs-All | 0.98 | 0.93 | 1.92 | 0.054 |
| MCS-Bacterial/Herberg et al. 2016 | Bacterial-vs-Viral | 0.95 | 0.93 | 0.95 | 0.34 |
| MCS-Viral/Herberg et al. 2016 | Viral-vs-All | 0.96 | 0.86 | 3.13 | 0.0018 |
| MCS-Bacterial/Herberg et al. 2016 | Bacterial-vs-All | 0.89 | 0.80 | 3.04 | 0.0024 |
| MCS-Kawasaki/Wright et al. 2016 | KD-vs All | 0.94 | 0.91 | 1.31 | 0.19 |

MCS: MultiClassSignature

### a Nine Gene Panel for Distinguishing Between and Assassin the Aetiology of Infectious and Inflammatory Diseases

There are multiple clinical contexts in which a diagnostic test which can distinguish multiple diseases simultaneously would be advantageous. The formulation of these biomarker panels would be dependent on clinical needs in a given context, the number of biomarkers which can be quantified using a given platform and the resulting predictive performance. In order to explore potential use cases for these types of biomarker panels, the inventors compared a variety of formulations using cross-validation in a dataset comprising whole blood RNA-Seq from a large number of patients with infectious and inflammatory diseases.

A maximum number of nine genes was used as a constraint for signature discovery to ensure easier translation to biomarker tests, and nine is a number more suitable for rapid point of care devices. When the inventors ran the analysis, they came up with a panel of nine genes that would be more suited for diagnostic purposes in a European emergency departments, where most patients have either bacterial or viral or inflammatory disease. This panel was able to distinguish bacterial infection, viral infection and inflammatory disease and was found to have in cross validation a mean one-versus-all AUROC (area under the receiver operating characteristic curve) of 0.9 for each disease.

A different set of genes was identified to address the multi-class diagnosis question in clinical contexts in Africa. In such a context, a test would need to consider tuberculosis and malaria as well, i.e. diseases that have high prevalence in Africa. In the data, a panel consisting of 9 genes could distinguish bacterial (with a one-versus-all AUROC of 0.8), viral (0.9), TB (0.8) and malaria (1). It could also distinguish bacterial from malaria (AUROC of 1), TB (0.8) and viral (0.9), malaria from TB (0.9) and viral (1) and TB from viral (0.9).

### Discussion and Conclusions

The inventors investigated whether multiple diseases could be simultaneously distinguished and identified using a single whole blood transcriptional panel. A multi-class machine learning approach was applied to publicly available blood gene expression datasets to identify a set of 161 transcripts sufficient for accurate diagnosis of diverse causes of febrile illness in children. The 161-transcript panel can identify 18 specific inflammatory diseases and pathogen species and distinguish between six broad disease categories (bacterial infection, viral infection, inflammatory disease, tuberculosis, malaria and Kawasaki disease). As some diagnostic errors carry severe consequences (such as failure to diagnose a life-threatening bacterial infection), while others have few adverse consequences (such as failing to diagnose a self-limiting viral infection for which there is no specific treatment), the inventors used a “cost-sensitive learning” approach in their discovery pipeline by example weighting. The inventors used a weighting scheme based on expert consensus which could effectively prioritise the predictions in favour of diseases for which misdiagnosis carries the greatest consequence. The 161-transcript signature identified using gene expression microarray data sets, was validated in a translated 145-gene form in an independent study of febrile children in whom gene expression levels were detected by RNA-Seq, supporting the clinical validity, the robustness, and the reproducibility of the approach.

This invention demonstrates that a single panel of RNA transcripts can be used to assign patients with fever and non-specific clinical and laboratory findings to a range of aetiologies from a single whole blood sample. Coupled with diagnostic technological advances able to measure RNA transcripts rapidly and at an affordable cost, a multi-class diagnostic test for febrile illness could circumvent lengthy clinical diagnostic processes, reduce delays to diagnoses, missed diagnoses, and unnecessary antibiotic treatment, having a significant impact on global health.

“The project leading to this application has received funding from the European Union's Horizon 2020 research and innovation programme under grant agreement No 668303”.

1. A method for diagnosing a subject a having at least one disease, the method comprising detecting, in a subject-derived RNA sample, a modulation in gene expression levels of a gene signature selected from the subject's transcriptome, simultaneously discriminating between at least three diseases and identifying the etiology of the at least one disease that the subject is suffering from, thereby diagnosing the subject a having the at least one disease.

2. The method according to claim 1, comprising simultaneously discriminating between at least four diseases.

3. The method according to either claim 1, comprising simultaneously discriminating between an infectious disease, an inflammatory disease, cancer, a metabolic disease, a degenerative disease, an endocrine disease and/or drug toxicity.

4. The method according to claim 3, wherein the infectious disease is a bacterial infection, a viral infection, or a parasitic infection.

5. The method according to claim 3, wherein when the subject is suffering from an infectious disease, the method comprises identifying the pathogen responsible for the infectious disease from which the subject is suffering from.

6. The method according to claim 3, wherein when the subject is suffering from an inflammatory disease, the method comprises naming the inflammatory disease at the disease or syndrome level.

7. The method according to claim 4, wherein the bacterial infection is selected from the group consisting of: Mycobacterium tuberculosis, Staphylococcus aureus, Escherichia coli, Group A Streptococcus, Streptococcus pneumoniae, Group B streptococcus, and Neisseria meningitidis.

8. The method according to claim 4, wherein the viral infection is selected from the group consisting of: influenza, Respiratory Syncytial Virus (RSV), enterovirus, adenovirus, human herpesvirus 6 (HHV6), and rhinovirus.

9. The method according to claim 4, wherein the parasitic infection is malaria.

10. The method according to claim 3, wherein the inflammatory disease is selected from the group consisting of: Kawasaki disease, systemic lupus erythematosus (SLE), juvenile idiopathic arthritis (JIA), and Henoch-Schonlein purpura (HSP).

11. The method according to claim 1, wherein the at least three diseases that are simultaneously discriminated between are not mutually exclusive.

12. The method according to claim 1, wherein the method comprises diagnosing a subject having at least two diseases, and identifying the etiology of the at least two diseases that the subject is suffering from, or wherein the method comprises diagnosing a subject having at least three diseases, and identifying the etiology of the at least three diseases that the subject is suffering from.

13. The method according to any one of the preceding claims claim 1, wherein the method comprises detecting the modulation in gene expression levels of at least 5 genes, at least 10 genes, at least 15 genes, at least 20 genes, at least 25 genes, at least 30 genes, at least 35 genes, at least 40 genes, at least 45 genes, or at least 50 genes, of a gene signature, or wherein the method comprises detecting the modulation in gene expression levels of at least 55 genes, at least 60 genes, at least 65 genes, at least 70 genes, at least 75 genes, at least 80 genes, at least 85 genes, at least 90 genes, at least 95 genes, or at least 100 genes, of the gene signature.

14. The method according to any one of the preceding claims claim 1, wherein the method comprises detecting the modulation in gene expression levels of at least 5 genes, at least 10 genes, at least 15 genes, at least 20 genes, at least 25 genes, at least 30 genes, at least 35 genes, at least 40 genes, at least 45 genes, or at least 50 genes, of a gene signature in Table 1, or wherein the method comprises detecting the modulation in gene expression levels of at least 55 genes, at least 60 genes, at least 65 genes, at least 70 genes, at least 75 genes, at least 80 genes, at least 85 genes, at least 90 genes, at least 95 genes, or at least 100 genes, of a gene signature in Table 1.

15. The method according to claim 1, wherein the method comprises detecting the modulation in gene expression levels of at least 105 genes, at least 110 genes, at least 115 genes, at least 120 genes, at least 125 genes, at least 130 genes, at least 135 genes, at least 140 genes, at least 145 genes, at least 150 genes, at least 155 genes, or at least 160 genes, or all 161 genes of a gene signature show in Table 1.

16. The method according to claim 1, wherein:
(i) when the at least one disease is influenza, the method comprises detecting the modulation in gene expression levels of at least two genes selected from the group consisting of: HESX1, IL4I1, AXL, IER5, RNASE1, NTN3, PID1, TEF, and C9orf21, and/or the method comprises detecting the modulation in gene expression levels with a probe comprising a nucleotide sequence as set out in SEQ ID No: 17;
(ii) when the at least one disease is respiratory syncytial virus (RSV), the method comprises detecting the modulation in gene expression levels of at least two genes selected from the group consisting of: HIST2H2BE, FLJ10213, PAK2, IFI6, ATP6V1G1, IFI27, EBI3, HNRPM, NOV, and PINX1;
(iii) when the at least one disease is adenovirus, the method comprises detecting the modulation in gene expression levels of at least two genes selected from the group consisting of: GLDC, BFSP2, IFI27, NOV, COQ10A, CHRM4, LOC648526, HSP90AA1, CLC, and MXRA7;
(iv) when the at least one disease is Kawasaki disease, the method comprises detecting the modulation in gene expression levels of at least two genes selected from the group consisting of: CCL23, APOL3, CETP, NTN3, ITGAX, HIST1H2BK, PRR5, MT1F, PLA2G7, and HIST1H3H;
(v) when the at least one disease is group A Streptococcus, the method comprises detecting the modulation in gene expression levels of at least two genes selected from the group consisting of: C9orf21, PARP8, GLDC, LOC644162, GIMAP4, MID1, IL8, LOC641705, HOXC10, and ITGAX;
(vi) when the at least one disease is juvenile idiopathic arthritis, the method comprises detecting the modulation in gene expression levels of at least two genes selected from the group consisting of: KIAA1600, NOV, PID1, NDRG2, LOC389816, HOXC10, CD24, EGLN2, IFI27, and BHLHB2;
(vii) when the at least one disease is systemic lupus erythematosus, the method comprises detecting the modulation in gene expression levels of at least two genes selected from the group consisting of: ZCCHC2, REC8, IFI27, NOV, IFI44L, IL8, TYSND1, TNRC6A, GAS6, and PIK3CG;
(viii) when the at least one disease is Human herpesvirus 6, the method comprises detecting the modulation in gene expression levels of at least two genes selected from the group consisting of: PARP8, TMED4, AC004080.2, MASTL, MXRA7, POLE4, HSP90AA1, PTPRM, MGC42367, and PRR5;
(ix) when the at least one disease is enterovirus, the method comprises detecting the modulation in gene expression levels of at least two genes selected from the group consisting of: RBM3, NPEPL1, GIMAP4, CDKN1C, RHOQ, TNFRSF21, IFI6, RTN1, LOC641705, and ITGAX;
(x) when the at least one disease is rhinovirus, the method comprises detecting the modulation in gene expression levels of at least two genes selected from the group consisting of: LOC652185, SEPN1, EBF2, ARHGEF9, CTAGE6, S100A9, C18orf54, HLA-A, TNFRSF21, and MMP9;
(xi) when the at least one disease is Escherichia coli, the method comprises detecting the modulation in gene expression levels of at least two genes selected from the group consisting of: BID, REC8, HIST1H3F, LHPP, CAV2, NPEPL1, IGLL1, PLA2G7, AMFR, and S100A9;
(xii) when the at least one disease is Staphylococcus aureus, the method comprises detecting the modulation in gene expression levels of at least two genes selected from the group consisting of: HOXA9, MS4A3, ATP6V1G1, DEM1, TEF, TXNRD2, BMF, SEPN1, PINX1, and IFI27;
(xiii) when the at least one disease is tuberculosis, the method comprises detecting the modulation in gene expression levels of at least two genes selected from the group consisting of: ARAP2, LOC644739, PRDM1, SNX6, RAP2C-AS1, TYSND1, PCGF6, EGLN2, and KCTD12, and/or the method comprises detecting the modulation in gene expression levels with a probe comprising a nucleotide sequence as set out in SEQ ID No: 17;
(xiv) when the at least one disease is malaria, the method comprises detecting the modulation in gene expression levels of at least two genes selected from the group consisting of: LOC650850, PDCD1, EBI3, TOMM70A, HSP90AA1, KIR3DL2, CTLA4, SAP18, MMP9, and VPS52;
(xv) when the at least one disease is Streptococcus pneumoniae, the method comprises detecting the modulation in gene expression levels of at least two genes selected from the group consisting of: RNASE3, COQ10A, DEM1, BRD2, VCX, CD24, HEMK1, TYSND1, CXCR6, and DEF6;
(xvi) when the at least one disease is group B Streptococcus, the method comprises detecting the modulation in gene expression levels of at least two genes selected from the group consisting of: VCX, ATP2C2, CYTSA, EXT2, MT1F, ARHGEF9, RHOQ, IGLL1, HLA-A, and P2RX7;
(xvii) when the at least one disease is Neisseria meningitidis, the method comprises detecting the modulation in gene expression levels of at least two genes selected from the group consisting of: CMTM1, RNASE1, MMD, AMACR, LOC644532, HEMK1, MTL5, C1QC, and CDKN1C, and/or the method comprises detecting the modulation in gene expression levels with a probe comprising a nucleotide sequence as set out in SEQ ID No: 161; and/or
(xviii) when the at least one disease is Henoch-Schonlein purpura, the method comprises detecting the modulation in gene expression levels of at least two genes selected from the group consisting of: C9orf117, APOL3, EPSTI1, SAP18, PCGF6, CYDL2, LOC648000, PID1, CHRM4, and CTAGE6.

17. The method according to claim 16, wherein:
(i) one or more of the following genes are up-regulated in a subject having influenza: HESX1, IL4I1, AXL, IER5, RNASE1, TEF and C9orf21, and/or one or more of the following genes are down-regulated in a subject having influenza: NTN3 and PID1;
(ii) one or more of the following genes are up-regulated in a subject having respiratory syncytial virus (RSV): IFI6, ATP6V1G1, IFI27, and HNRPM, and/or one or more of the following genes are down-regulated in a subject having respiratory syncytial virus (RSV): HIST2H2BE, FLJ10213, PAK2, EBI3, NOV, and PINX1;
(iii) one or more of the following genes are up-regulated in a subject having adenovirus: GLDC, BFSP2, IFI27, COQ10A, CHRM4, HSP90AA1, and MXRA7, and/or one or more of the following genes are down-regulated in a subject having adenovirus: NOV, LOC648526, and CLC;
(iv) one or more of the following genes are up-regulated in a subject having Kawasaki disease: CCL23, CETP, NTN3, ITGAX, HIST1H2BK, and HIST1H3H, and/or one or more of the following genes are down-regulated in a subject having Kawasaki disease: APOL3, PRR5, MT1F, and PLA2G7;
(v) one or more of the following genes are up-regulated in a subject having group A Streptococcus: GLDC and ITGAX, and/or one or more of the following genes are down-regulated in a subject having group A Streptococcus: C9orf21, PARP8, LOC644162, GIMAP4, MID1, IL8, LOC641705, and HOXC10;
(vi) one or more of the following genes are up-regulated in a subject having juvenile idiopathic arthritis: NOV, PID1, NDRG2, LOC389816, HOXC10, EGLN2, and BHLHB2, and/or one or more of the following genes are down-regulated in a subject having juvenile idiopathic arthritis: KIAA1600, CD24, and IFI27;
(vii) one or more of the following genes are up-regulated in a subject having systemic lupus erythematosus: ZCCHC2, REC8, IFI27, NOV, IL8, IFI44L, TYSND1, and TNRC6A, and/or one or more of the following genes are down-regulated in a subject having systemic lupus erythematosus: GAS6 and PIK3CG;
(viii) one or more of the following genes are up-regulated in a subject having Human herpesvirus 6: PARP8, TMED4, AC004080.2, MASTL, MXRA7, POLE4, HSP90AA1, and PRR5, and/or one or more of the following genes are down-regulated in a subject having Human herpesvirus 6: PTPRM and MGC42367;
(ix) one or more of the following genes are up-regulated in a subject having enterovirus: GIMAP4, CDKN1C, TNFRSF21, and IFI6, and/or one or more of the following genes are down-regulated in a subject having enterovirus: RBM3, NPEPL1, RHOQ, RTN1, LOC641705, and ITGAX;
(x) one or more of the following genes are up-regulated in a subject having rhinovirus: LOC652185, ARHGEF9, C18orf54, EBF2, and TNFRSF21, and/or one or more of the following genes are down-regulated in a subject having rhinovirus: SEPN1, CTAGE6, S100A9, HLA-A, and MMP9;
(xi) one or more of the following genes are up-regulated in a subject having Escherichia coli: BID, CAV2, PLA2G7, and S100A, and/or one or more of the following genes are down-regulated in a subject having Escherichia coli: REC8, HIST1H3F, LHPP, NPEPL1, IGLL1, and AMFR;
(xii) one or more of the following genes are up-regulated in a subject having Staphylococcus aureus: HOXA9, MS4A3, TEF, TXNRD2, BMF, and SEPN1, and/or one or more of the following genes are down-regulated in a subject having Staphylococcus aureus: ATP6V1G1, DEM1, PINX1, and IFI27;
(xiii) one or more of the following genes are up-regulated in a subject having tuberculosis: ARAP2, LOC644739, PRDM1, PCGF6, RAP2C-AS1, and KCTD12, and/or one or more of the following genes are down-regulated in a subject having tuberculosis: SNX6, TYSND1, and EGLN2;
(xiv) one or more of the following genes are up-regulated in a subject having malaria: LOC650850, PDCD1, EBI3, TOMM70A, HSP90AA1, KIR3DL2, CTLA4, SAP18, MMP9, and VPS52;
(xv) one or more of the following genes are up-regulated in a subject having Streptococcus pneumoniae: RNASE3, VCX, and CD24, and/or one or more of the following genes are down-regulated in a subject having Streptococcus pneumoniae: COQ10A, DEM1, BRD2, HEMK1, TYSND1, CXCR6, and DEF6;
(xvi) one or more of the following genes are up-regulated in a subject having group B Streptococcus: VCX, ATP2C2, and CYTSA, and/or one or more of the following genes are down-regulated in a subject having group B Streptococcus: EXT2, MT1F, ARHGEF9, RHOQ, IGLL1, HLA-A, and P2RX7;
(xvii) one or more of the following genes are up-regulated in a subject having Neisseria meningitidis: CMTM1, RNASE1, AMACR, LOC644532, HEMK1, MTL5, C1QC, and CDKN1C, and/or the gene MMD is down-regulated in a subject having Neisseria meningitidis; and/or
(xviii) one or more of the following genes are up-regulated in a subject having Henoch-Schonlein purpura: PCGF6, LOC648000, PID1, and CHRM4, and/or one or more of the following genes are down-regulated in a subject having Henoch-Schonlein purpura: C9orf117, APOL3, EPSTI1, SAP18, CYDL2, and CTAGE6.

18. The method according to claim 1, further comprising determining the likelihood that the subject suffers from the at least one disease.

19. A diagnostic kit for diagnosing a subject as having at least one disease, by simultaneously discriminating between at least three diseases and identifying the etiology of the at least one disease that the subject is suffering from, the kit comprising oligonucleotides for detecting, in a subject-derived RNA sample, the modulation in gene expression levels of at least two genes selected from a gene signature in Table 1.

20. A diagnostic or prognostic biomarker for at least one disease comprising at least two genes selected from a gene signature in Table 1.

21. A gene chip comprising probes for detecting the modulation in gene expression levels of at least two genes of a gene signature in Table 1.
